SEQUENCE LISTING (14 sequences)

SEQ ID NO: 1 - protein, 767 aa, Homo sapiens
MAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 3 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 4 - DNA, 22 bp, Artificial Sequence
  Description of Artificial Sequence Synthetic primer
agttttcgttttcgtagtcgtc

SEQ ID NO: 5 - DNA, 19 bp, Artificial Sequence
  Description of Artificial Sequence Synthetic primer
cgaatataacctcaccccg

SEQ ID NO: 6 - DNA, 24 bp, Artificial Sequence
  Description of Artificial Sequence Synthetic primer
ggagtttttgtttttgtagttgtt

SEQ ID NO: 7 - DNA, 22 bp, Artificial Sequence
  Description of Artificial Sequence Synthetic primer
aaccaaatataacctcacccca

SEQ ID NO: 8 - DNA, 21 bp, Artificial Sequence
  Description of Artificial Sequence Synthetic primer
ggtgtggtggatgtgagattt

SEQ ID NO: 9 - DNA, 22 bp, Artificial Sequence
  Description of Artificial Sequence Synthetic primer
ctttcatcacccacaaactaac

SEQ ID NO: 10 - DNA, 21 bp, Artificial Sequence
  Description of Artificial Sequence Synthetic primer
atagggaggagattagggttt

SEQ ID NO: 11 - DNA, 23 bp, Artificial Sequence
  Description of Artificial Sequence Synthetic primer
gggaggtttgtttgtgtttaagg

SEQ ID NO: 12 - DNA, 26 bp, Artificial Sequence
  Description of Artificial Sequence Synthetic primer
tctgggcctacattgctaaaatctaa

SEQ ID NO: 13 - DNA, 26 bp, Artificial Sequence
  Description of Artificial Sequence Synthetic primer
gttgagaccttcaatgactttctagt

SEQ ID NO: 14 - DNA, 13 bp, Homo sapiens
ctacagtgaaatc

(19) United States
(12) Patent Application Publication
(10) Pub. No.: US 2021/0332442 A1
(43) Pub. Date: Oct. 28, 2021

(54) NON-INVASIVE SKIN-BASED DETECTION METHODS

(71) Applicant: DERMTECH, INC., La Jolla, CA (US)

(72) Inventors: John Daniel DOBAK, III, La Jolla, CA (US); Burkhard JANSEN, La Jolla, CA (US); Zuxu YAO, San Diego, CA (US)

(21) Appl. No.: 17/354,899

(22) Filed: Jun. 22, 2021

Related U.S. Application Data
(63) Continuation of application No. 16/603,435, filed on Oct. 7, 2019.
(60) Provisional application No. 62/483,834, filed on Apr. 10, 2017.
(60) Provisional application No. 62/562,250, filed on Sep. 22, 2017.

(51) Int. Cl. C12Q 1/6886; A61B 10/02; C12Q 1/6806; C12Q 1/6888

(52) U.S. Cl. CPC C12Q 1/6886; A61B 10/02; C12Q 1/6806; C12Q 2600/158; C12Q 2600/154; C12Q 2600/156; C12Q 1/6888

(57) ABSTRACT

Disclosed herein are analytical methods and compositions for detecting expression level and mutational change in an individual in need thereof, which profiles RNA, genomic DNA, and/or microbial DNA. Also described herein include diagnostic methods which are based on the changes of expression levels and mutational change of RNA, genomic DNA, and/or microbial DNA.

### CROSS-REFERENCE

This application is a continuation of U.S. application Ser. No. 16/603,435, filed Oct. 7, 2019, which is a § 371 U.S. national stage entry of International Application No. PCT/US2018/026902 filed Apr. 10, 2018, which claims the benefit of U.S. Provisional Application No. 62/483,834, filed Apr. 10, 2017, and U.S. Provisional Application No. 62/562,250, filed Sep. 22, 2017, each of which is incorporated herein by reference.

### SEQUENCE LISTING

The instant application contains a Sequence Listing which has been submitted electronically in ASCII format and is hereby incorporated by reference in its entirety. Said ASCII copy, created Jun. 22, 2021, is named 44503-720_302_SL.txt and is 20,680 bytes in size.

### BACKGROUND

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability.

### SUMMARY

An aspect described herein is an analysis method of detecting expression level and mutational change in an individual in need thereof, comprising: (a) contacting a biological sample with a plurality of beads; (b) co-isolating RNA and genomic DNA from the plurality of beads; (c) amplifying both the RNA and genomic DNA extracted from step (b); (d) detecting the expression level of a RNA of interest from the RNA isolated from the beads; and (e) detecting the mutational change of a gene of interest from the genomic DNA isolated from the beads. In one feature, the plurality of beads is a plurality of silica-coated beads. In one feature, the plurality of silica-coated beads is a plurality of silica-coated magnetic beads. the biological sample comprises a blood sample, saliva sample, urine sample, serum sample, plasma sample, tear sample, skin sample, tissue sample, hair sample, sample from cellular extracts, or a tissue biopsy sample. In one feature, the biological sample comprises a skin sample. In one feature, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In one feature, the lesion is suspected to be melanoma.

In one feature, the skin sample comprises keratinocytes, melanocytes, basal cells, T-cells, or dendritic cells. In one feature, the biological sample comprises prokaryotic nucleic acid material. In one feature, the RNA is mRNA. In one feature, the RNA is cell-free circulating RNA. In one feature, the genomic DNA is cell-free circulating genomic DNA. In one feature, the biological sample is obtained by applying a plurality of adhesive patches to a skin sample in a manner sufficient to adhere a sample of the skin to the adhesive patch, and removing the adhesive patch from the skin in a manner sufficient to retain the adhered skin sample to the adhesive patch. In one feature, the plurality of adhesive patches comprises at least 4 adhesive patches. In one feature, the plurality of adhesive patches comprises about 4 adhesive patches. In one feature, the biological sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of skin sample is removed by the plurality of adhesive patches. In one feature, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In one feature, the RNA or DNA is stable on the plurality of adhesive patches for at least 1 week. In one feature, the RNA or DNA is stable on the plurality of adhesive patches at a temperature of up to about 60° C. In one feature, the RNA or DNA is stable on the plurality of adhesive patches at room temperature. In one feature, a yield of RNA or DNA is at least about 200 picograms, at least about 500 picograms, at least about 750 picograms, at least about 1000 picograms, at least about 1500 picograms, or at least about 2000 picograms. In one feature, detecting gene expression of RNA comprises quantitative polymerase chain reaction (qPCR), RNA sequencing, or microarray analysis. In one feature, the gene expression is of LINC, PRAME, DNMT1, DNMT3A, DNMT3B, DNMT3L, KRT1, KRT10, IVL, or TGase5. In one feature, detecting mutational change in the DNA comprises allele specific polymerase chain reaction (PCR) or a sequencing reaction. In one feature, the gene of interest comprises NF1, TERT, CDKN2a, NRAS, KRAS, HRAS, BRAF, KIT PTEN, TP53, ARID1A, ARID1B, or ARID2. In one feature, the mutational change comprises a mutation in NF1, TERT, CDKN2a, NRAS, KRAS, HRAS, BRAF, KIT, PTEN, TP53, ARID1A, ARID1B, or ARID2. In one feature, the mutational change comprises a mutation in TERT, NRAS, or BRAF. In one feature, the mutational change comprises a mutation in at least two genes selected from a list consisting of TERT, NRAS, and BRAF. In one feature, the method further comprises isolating microbial DNA and/or microbial RNA. In one feature, the microbial DNA and/or microbial RNA is isolated from a skin sample. In one feature, the microbial DNA and/or microbial RNA is isolated from the epidermis layer. In one feature, the microbial DNA and/or microbial RNA is isolated from the dermis layer.

An aspect described herein is a method of detecting expression level and mutational change in an individual in need thereof, comprising: (a) obtaining a skin sample using a plurality of adhesive patches; (b) contacting the skin sample with a plurality of beads; (c) co-isolating RNA and genomic DNA from the plurality of beads; (d) amplifying both the RNA and genomic DNA extracted from step (c); (e) detecting the expression level of a RNA of interest from the RNA of step (d); and (f) detecting the mutational change of a gene of interest from the genomic DNA of step (d). In one feature, the plurality of beads is a plurality of silica-coated beads. In one feature, the plurality of silica-coated beads is a plurality of silica-coated magnetic beads. In one feature, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In one feature, the lesion is suspected to be melanoma. In one feature, the skin sample comprises keratinocytes, melanocytes, basal cells, T-cells, or dendritic cells. In one feature, the skin sample comprises prokaryotic nucleic acid material. In one feature, the RNA is mRNA. In one feature, the RNA is cell-free circulating RNA. In one feature, the genomic DNA is cell-free circulating genomic DNA. In one feature, the plurality of adhesive patches comprises at least 4 adhesive patches. In one feature, the plurality of adhesive patches comprises about 4 adhesive patches. In one feature, the skin sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of skin sample is removed by the plurality of adhesive patches. In one feature, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In one feature, the RNA or DNA is stable on the plurality of adhesive patches for at least 1 week. In one feature, the RNA or DNA is stable on the plurality of adhesive patches at a temperature of up to about 60° C. In one feature, the RNA or DNA is stable on the plurality of adhesive patches at room temperature. In one feature, a yield of RNA or DNA is at least about 200 picograms, at least about 500 picograms, at least about 750 picograms, at least about 1000 picograms, at least about 1500 picograms, or at least about 2000 picograms. In one feature, detecting gene expression of RNA comprises quantitative polymerase chain reaction (qPCR), RNA sequencing, or microarray analysis. In one feature, the gene expression is of LINC, PRAME, DNMT1, DNMT3A, DNMT3B, DNMT3L, KRT1, KRT10, IVL, or TGase5. In one feature, detecting mutational change in the DNA comprises allele specific polymerase chain reaction (PCR) or a sequencing reaction. In one feature, the gene of interest comprises NF1, TERT, CDKN2a, NRAS, KRAS, HRAS, BRAF, KIT, PTEN, TP53, ARID1A, ARID1B, or ARID2. In one feature, the mutational change comprises a mutation in NF1, TERT, CDKN2a, NRAS, KRAS, HRAS, BRAF, KIT, PTEN, TP53, ARID1A, ARID1B, or ARID2. In one feature, the mutational change comprises a mutation in TERT, NRAS, or BRAF. In one feature, the mutational change comprises a mutation in at least two genes selected from a list consisting of TERT, NRAS, and BRAF. In one feature, the method further comprises isolating microbial DNA and/or microbial RNA. In one feature, the microbial DNA and/or microbial RNA is isolated from a skin sample. In one feature, the microbial DNA and/or microbial RNA is isolated from the epidermis layer. In one feature, the microbial DNA and/or microbial RNA is isolated from the epidermal layer.

An aspect described herein is a method of diagnosing a disease or disorder in an individual, comprising: (a) contacting a biological sample with a plurality of beads; (b) co-isolating RNA and genomic DNA from the plurality of beads; (c) amplifying both the RNA and genomic DNA extracted from step (b); (d) detecting an expression level of a RNA of interest from the RNA of step (c) and comparing the expression level to a control; (e) detecting a mutational change of a gene of interest from the genomic DNA; and (f) based on step d) and e), diagnosing the individual as having a disease or disorder if there is a change in the expression level of the RNA of interest relative to the control and the presence of the mutational change in the gene of interest. An aspect described herein is a method of diagnosing a disease or disorder in an individual, comprising: (a) obtaining a skin sample using a plurality of adhesive patches; (b) contacting the skin sample with a plurality of beads; (c) co-isolating RNA and genomic DNA from the plurality of beads; (d) amplifying both the RNA and genomic DNA extracted from step (c); (e) detecting an expression level of a RNA of interest from the RNA of step (d) and comparing the expression level to a control; (f) detecting a mutational change of a gene of interest from the genomic DNA of step (d); and (g) identifying the individual as having the disease or disorder by comparing the gene expression and the mutational change to a control, wherein a change in the expression level of the RNA of interest relative to the control and the presence of the mutational change of the gene of interest indicate the presence of a disease or disorder in the individual. An aspect described herein is a method of diagnosing a disease or disorder in an individual, comprising: (a) co-isolating RNA and genomic DNA from a skin sample; (b) amplifying both the RNA and genomic DNA extracted from step (a); (c) detecting an expression level of a RNA of interest from the RNA of step (b) and comparing the expression level to a control; (d) detecting a mutational change of a gene of interest from the genomic DNA; and based on step c) and d), diagnosing the individual as having a disease or disorder if there is a change in the expression level of the RNA of interest relative to the control and the presence of the mutational change in the gene of interest. In one feature, the biological sample comprises a blood sample, saliva sample, urine sample, serum sample, plasma sample, tear sample, skin sample, tissue sample, hair sample, sample from cellular extracts, or a tissue biopsy sample. In one feature, the biological sample comprises a skin sample. In one feature, the plurality of beads is a plurality of silica-coated beads. In one feature, the plurality of silica-coated beads is a plurality of silica-coated magnetic beads. In one feature, the disease or disorder comprises melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In one feature, wherein the skin sample comprises a lesion, and wherein the lesion is suspected to be a melanoma. In one feature, the skin sample comprises keratinocytes, melanocytes, basal cells, T-cells, or dendritic cells. In one feature, the biological sample comprises prokaryotic nucleic acid material. In one feature, the skin sample comprises prokaryotic nucleic acid material. In one feature, the RNA is mRNA. In one feature, the RNA is cell-free circulating RNA. In one feature, the genomic DNA is cell-free circulating genomic DNA. In one feature, the control is a sample from a healthy individual. In one feature, the control is a sample from an individual with a known disease or disorder. In one feature, the control is a normal sample from the same individual. In one feature, the biological sample is obtained by applying a plurality of adhesive patches to a skin sample in a manner sufficient to adhere a sample of the skin to the adhesive patch, and removing the adhesive patch from the skin in a manner sufficient to retain the adhered skin sample to the adhesive patch. In one feature, the plurality of adhesive patches comprises at least 4 adhesive patches. In one feature, the plurality of adhesive patches comprises about 4 adhesive patches. In one feature, the biological sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of skin sample is removed by the plurality of adhesive patches. In one feature, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In one feature, the RNA or DNA is stable on the plurality of adhesive patches for at least 1 week. In one feature, the RNA or DNA is stable on the plurality of adhesive patches at a temperature of up to about 60° C. In one feature, the RNA or DNA is stable on the plurality of adhesive patches at room temperature. In one feature, a yield of RNA or DNA is at least about 200 picograms, at least about 500 picograms, at least about 750 picograms, at least about 1000 picograms, at least about 1500 picograms, or at least about 2000 picograms. In one feature, detecting gene expression of RNA comprises quantitative polymerase chain reaction (qPCR), RNA sequencing, or microarray analysis. In one feature, the gene expression is of LINC, PRAME, DNMT1, DNMT3A, DNMT3B, DNMT3L, KRT1, KRT10, IVL, or TGase5. In one feature, detecting mutational change in the DNA comprises allele specific polymerase chain reaction (PCR) or a sequencing reaction. In one feature, the gene of interest comprises NF1, TERT, CDKN2a, NRAS, KRAS, HRAS, BRAF, KIT, PTEN, TP53, ARID1A, ARID1B, or ARID2. In one feature, the mutational change comprises a mutation in NF1, TERT, CDKN2a, NRAS, KRAS, HRAS, BRAF, KIT, PTEN, TP53, ARID1A, ARID1B, or ARID2. In one feature, the mutational change comprises a mutation in TERT, NRAS, or BRAF. In one feature, the mutational change comprises a mutation in at least two genes selected from a list consisting of TERT, NRAS, and BRAF. In one feature, the individual is diagnosed as having a disease or disorder when the biological sample: is positive for PRAME, LINC, or a combination thereof; and comprises one or more mutations in NF1, TERT, CDKN2a, NRAS, KRAS, HRAS, BRAF, KIT PTEN, TP53, ARID1A, ARID1B,ARID2, or a combination thereof. In one feature, the individual is diagnosed as having the disease or disorder when the biological sample: is positive for PRAME, LINC, or a combination thereof; and comprises one or more mutations in at least two genes selected from a list consisting of NF1, TERT, CDKN2a, NRAS, KRAS, HRAS, BRAF, KIT, PTEN, TP53, ARID1A, ARID1B, and ARID2. In one feature, the individual is diagnosed as having the disease or disorder when the biological sample: is positive for PRAME, LINC, or a combination thereof; and comprises one or more mutations in TERT, NRAS, BRAF, or a combination thereof. In one feature, the individual is diagnosed as having the disease or disorder when the biological sample: is positive for PRAME, LINC, or a combination thereof; and comprises one or more mutations in at least two genes selected from a list consisting of TERT, NRAS, and BRAF. In one feature, the mutational change comprises a mutation in BRAF and a mutation in NRAS. In one feature, the mutational change comprises a mutation in BRAF and a mutation in TERT. In one feature, the mutational change comprises a mutation in NRAS and a mutation in TERT. In one feature, the mutational change comprises a mutation in TERT. In one feature, the mutational change comprises at least 1.5×, 2×, 3×, 4×, 5×, 6×, 7×, 8×, 9×, 10×, 11×, or 12× more mutations in NF1, TERT, CDKN2a, NRAS, KRAS, HRAS, BRAF, KIT, PTEN, TP53, ARID1A, ARID1B, ARID2, or a combination thereof, compared to a normal biological sample. In one feature, the mutational change comprises at least 1.5×, 2×, 3×, 4×, 5×, 6×, 7×, 8×, 9×, 10×, 11×, or 12× more mutations in TERT, NRAS, BRAF, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in TERT, NRAS, BRAF, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in NF1, TERT, CDKN2a, NRAS, KRAS, HRAS, BRAF, KIT, PTEN, TP53, ARID1A, ARID1B,ARID2, or a combination thereof, compared to a normal biological sample. In one feature, the method further comprises isolating microbial DNA and/or microbial RNA. In one feature, the microbial DNA and/or microbial RNA is isolated from a skin sample. In one feature, the microbial DNA and/or microbial RNA is isolated from the epidermis layer. In one feature, the microbial DNA and/or microbial RNA is isolated from the dermis layer.

An aspect described herein is a method for evaluating an individual for risk of developing a disease or disorder comprising: (a) measuring gene expression and mutational change in a skin sample from the individual; (b) comparing the gene expression and the mutational change to a control; and (c) identifying the individual as having or not having a risk factor for developing the disease or disorder based on a comparison of the gene expression and the mutational change measured in step (a) to the control, wherein the risk factor is determined if the gene expression and mutational change is different than the control. In one feature, the skin sample is obtained using a plurality of adhesive patches. In one feature, gene expression is measured from RNA obtained from the skin sample. In one feature, mutational change is measured from DNA obtained from the skin sample. In one feature, the RNA is isolated using a plurality of beads. In one feature, the DNA is isolated using a plurality of beads. In one feature, the plurality of beads is a plurality of silica-coated beads. In one feature, the plurality of silica-coated beads is a plurality of silica-coated magnetic beads. In one feature, the skin sample is suspicious for melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In one feature, the skin sample is suspicious for melanoma. In one feature, the skin sample comprises keratinocytes, melanocytes, basal cells, T-cells, or dendritic cells. In one feature, the skin sample comprises prokaryotic nucleic acid material. In one feature, the control is a sample from a healthy individual. In one feature, the control is a sample from an individual with a known disease or disorder. In one feature, the control is a normal sample from the same individual. In one feature, the RNA is mRNA. In one feature, the RNA is cell-free circulating RNA. In one feature, the DNA is cell-free circulating DNA. In one feature, the plurality of adhesive patches comprises at least 4 adhesive patches. In one feature, the plurality of adhesive patches comprises about 4 adhesive patches. In one feature, the skin sample is obtained by pooling the plurality of adhesive patches. In one feature, each adhesive patch of the plurality of adhesive patches is used separately. In one feature, each adhesive patch of the plurality of adhesive patches is circular. In one feature, the each adhesive patch is at least 19 mm in diameter. In one feature, the each adhesive patch is about 19 mm in diameter. In one feature, an effective amount of skin sample is removed by the plurality of adhesive patches. In one feature, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In one feature, the RNA or DNA is stable on the plurality of adhesive patches for at least 1 week. In one feature, the RNA or DNA is stable on the plurality of adhesive patches at a temperature of up to about 60° C. In one feature, the RNA or DNA is stable on the plurality of adhesive patches at room temperature. In one feature, a yield of RNA or DNA is at least about 200 picograms, at least about 500 picograms, at least about 750 picograms, at least about 1000 picograms, at least about 1500 picograms, or at least about 2000 picograms. In one feature, measuring gene expression comprises quantitative polymerase chain reaction (qPCR), RNA sequencing, or microarray analysis. In one feature, the gene expression is LINC, PRAME, DNMT1, DNMT3A, DNMT3B, DNMT3L, KRT1, KRT10, IVL, or TGase5. In one feature, measuring mutational change comprises allele specific polymerase chain reaction (PCR) or a sequencing reaction. In one feature, the gene of interest comprises NF1, TERT, CDKN2a, NRAS, KRAS, HRAS, BRAF, KIT, PTEN, TP53, ARID1A, ARID1B, or ARID2. In one feature, the mutational change comprises a mutation in NF1, TERT, CDKN2a, NRAS, KRAS, HRAS, BRAF, KIT, PTEN, TP53, ARID1A, ARID1B, or ARID2. In one feature, the mutational change comprises a mutation in TERT, NRAS, or BRAF. In one feature, the mutational change comprises a mutation in at least two genes selected from a list consisting of TERT, NRAS, and BRAF. In one feature, the individual is identified as having the risk factor for developing the disease or disorder when the skin sample: is positive for PRAME, LINC, or a combination thereof; and comprises one or more mutations in NF1, TERT, CDKN2a, NRAS, KRAS, HRAS, BRAF, KIT, PTEN, TP53, ARID1A, ARID1B, ARID2, or a combination thereof. In one feature, the individual is identified as having the risk factor for developing the disease or disorder when the skin sample: is positive for PRAME, LINC, or a combination thereof; and comprises one or more mutations in at least two genes selected from a list consisting of NF1, TERT, CDKN2a, NRAS, KRAS, HRAS, BRAF, KIT, PTEN, TP53, ARID1A, ARID1B, and ARID2. In one feature, the individual is identified as having the risk factor for developing the disease or disorder when the skin sample: is positive for PRAME, LINC, or a combination thereof; and comprises one or more mutations in TERT, NRAS, BRAF, or a combination thereof. In one feature, the individual is identified as having the risk factor for developing the disease or disorder when the skin sample: is positive for PRAME, LINC, or a combination thereof; and comprises one or more mutations in at least two genes selected from a list consisting of TERT, NRAS, and BRAF. In one feature, the mutational change comprises a mutation in BRAF and a mutation in NRAS. In one feature, the mutational change comprises a mutation in BRAF and a mutation in TERT. In one feature, the mutational change comprises a mutation in NRAS and a mutation in TERT. In one feature, the mutational change comprises a mutation in TERT. In one feature, the mutational change comprises at least 1.5×, 2X, 3×, 4×, 5×, 6×, 7×, 8×, 9×, 10×, 11×, or 12× more mutations in NF1, TERT, CDKN2a, NRAS, KRAS, HRAS, BRAF, KIT, PTEN, TP53, ARID1A, ARID1B, ARID2, or a combination thereof, compared to a normal biological sample. In one feature, the mutational change comprises at least 1.5×, 2×, 3×, 4×, 5×, 6×, 7×, 8×, 9×, 10×, 11×, or 12× more mutations in TERT, NRAS, BRAF, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in TERT, NRAS, BRAF, or a combination thereof, compared to a normal biological sample. In one feature, the mutational change comprises at least 10%, 15%, 20%, 25%, 30%, 35%, 40%, 45%, 50%, 55%, 60%, 65%, 70%, 75%, or 80% more mutations in NF1, TERT, CDKN2a, NRAS, KRAS, HRAS, BRAF, KIT, PTEN, TP53, ARID1A, ARID1B,ARID2, or a combination thereof, compared to a normal biological sample. In one feature, the method further comprises isolating microbial DNA and/or microbial RNA. In one feature, the microbial DNA and/or microbial RNA is isolated from a skin sample. In one feature, the microbial DNA and/or microbial RNA is isolated from the epidermis layer. In one feature, the microbial DNA and/or microbial RNA is isolated from the dermis layer. In one feature, a sensitivity of the method is at least 95%. In one feature, a specificity of the method is at least 90%.

Disclosed herein, in certain embodiments, is a method of detecting nucleic acid expression level and modification in a biological sample, comprising: (a) contacting the biological sample obtained from an individual in need thereof with a plurality of beads; (b) co-isolating RNA and genomic DNA from the plurality of beads; (c) amplifying both the RNA and genomic DNA extracted from step (b); (d) detecting the expression level of a RNA of interest from the RNA isolated from the beads; and (e) detecting a mutational change, a methylation status, or a combination thereof from a gene of interest from the genomic DNA isolated from the beads. In some embodiments, the plurality of beads is a plurality of silica-coated beads. In some embodiments, the plurality of silica-coated beads is a plurality of silica-coated magnetic beads. In some embodiments, the biological sample comprises a blood sample, saliva sample, urine sample, serum sample, plasma sample, tear sample, skin sample, tissue sample, hair sample, sample from cellular extracts, or a tissue biopsy sample. In some embodiments, the skin sample comprises a lesion. In some embodiments, the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In some embodiments, the skin sample comprises keratinocytes, melanocytes, basal cells, T-cells, or dendritic cells. In some embodiments, the biological sample comprises prokaryotic nucleic acid material. In some embodiments, the RNA comprises mRNA, cell-free circulating RNA, or a combination thereof. In some embodiments, the genomic DNA comprises cell-free circulating genomic DNA. In some embodiments, the skin sample is obtained by applying a plurality of adhesive patches to a skin region in a manner sufficient to adhere a sample of the skin to the adhesive patch, and removing the adhesive patch from the skin in a manner sufficient to retain the adhered skin sample to the adhesive patch. In some embodiments, the plurality of adhesive patches comprises at least 4 adhesive patches. In some embodiments, the biological sample is obtained by pooling the plurality of adhesive patches. In some embodiments, each adhesive patch of the plurality of adhesive patches is used separately to obtain a sample at a different skin depth. In some embodiments, an effective amount of skin sample is removed by the plurality of adhesive patches. In some embodiments, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In some embodiments, the RNA or DNA is stable on the plurality of adhesive patches for at least 1 week. In some embodiments, the RNA or DNA is stable on the plurality of adhesive patches at a temperature of up to about 60° C. In some embodiments, the RNA or DNA is stable on the plurality of adhesive patches at room temperature. In some embodiments, a yield of RNA or DNA from the biological sample is at least about 200 picograms, at least about 500 picograms, at least about 750 picograms, at least about 1000 picograms, at least about 1500 picograms, or at least about 2000 picograms. In some embodiments, detecting gene expression of RNA comprises quantitative polymerase chain reaction (qPCR), RNA sequencing, or microarray analysis. In some embodiments, the gene expression is of LINC, PRAME, DNMT1, DNMT3A, DNMT3B, DNMT3L, KRT1, KRT10, IVL, or TGase5. In some embodiments, the gene expression level is determined by contacting the biological sample with a set of probes that hybridizes to LINC, PRAME, DNMT1, DNMT3A, DNMT3B, DNMT3L, KRT1, KRT10, IVL, or TGase5, and detect binding between LINC, PRAME, DNMT1, DNMT3A, DNMT3B, DNMT3L, KRT1, KRT10, IVL, or TGase5 and the set of probes. In some embodiments, the gene expression level is determined by contacting the biological sample with a set of probes that hybridizes to one and no more than ten genes selected from: LINC, PRAME, DNMT1, DNMT3A, DNMT3B, DNMT3L, KRT1, KRT10, IVL, or TGase5 and detect binding between LINC, PRAME, DNMT1, DNMT3A, DNMT3B, DNMT3L, KRT1, KRT10, IVL, or TGase5 and the set of probes. In some embodiments, detecting mutational change in the DNA comprises allele specific polymerase chain reaction (PCR) or a sequencing reaction. In some embodiments, the mutational change comprises: a mutation in NF1, TERT, CDKN2a, NRAS, KRAS, HRAS, BRAF, KIT, PTEN, TP53, ARID1A, ARID1B, or ARID2; a mutation in TERT, NRAS, or BRAF; a mutation in at least two genes selected from a list consisting of TERT, NRAS, and BRAF; a mutation in BRAF and a mutation in NRAS; a mutation in BRAF and a mutation in TERT; a mutation in NRAS and a mutation in TERT; or a mutation in TERT. In some embodiments, the methylation status is detected in KRT10, KRT14, KRT15, KRT80, or a combination thereof. In some embodiments, the expression level of HNC, PRAME, DNMT1, DNMT3A, DNMT3B, DNMT3L, KRT1, KRT10, IVL, TGase5, or a combination thereof is detected and the methylation status of KRT10, KRT14, KRT15, KRT80, or a combination thereof is detected. In some embodiments, the individual is further diagnosed as having a disease or disorder, when the biological sample: is positive for PRAME, LINC, or a combination thereof; and comprises one or more mutations in NF1, TERT, CDKN2a, NRAS, KRAS, HRAS, BRAF, KIT, PTEN, TP53, ARID1A, ARID1B, ARID2, or a combination thereof. In some embodiments, the individual is diagnosed as having the disease or disorder when the biological sample: is positive for PRAME, LINC, or a combination thereof; and comprises one or more mutations in at least two genes selected from a list consisting of NF1, TERT, CDKN2a, NRAS, KRAS, HRAS, BRAF, KIT, PTEN, TP53, ARID1A, ARID1B, and ARID2. In some embodiments, the individual is diagnosed as having the disease or disorder when the biological sample: is positive for PRAME, LINC, or a combination thereof; and comprises one or more mutations in TERT, NRAS, BRAF, or a combination thereof. In some embodiments, the individual is diagnosed as having the disease or disorder when the biological sample: is positive for PRAME, LINC, or a combination thereof; and comprises one or more mutations in at least two genes selected from a list consisting of TERT, NRAS, and BRAF. In some embodiments, the mutational change comprises: at least 1.5×, 2×, 3×, 4×, 5×, 6×, 7×, 8×, 9×, 10×, 11×, or 12× more mutations in NF1, TERT, CDKN2a, NRAS, KRAS, HRAS, BRAF, KIT, PTEN, TP53, ARID1A, ARID1B, ARID2, or a combination thereof, compared to a normal biological sample; or at least 1.5×, 2×, 3×, 4×, 5×, 6×, 7×, 8×, 9×, 10×, 11×, or 12× more mutations in TERT, NRAS, BRAF, or a combination thereof, compared to a normal biological sample. In some embodiments, the mutational change comprises: at least 10%, 15%, 20%, 25%, 30%, 35%, 40%, 45%, 50%, 55%, 60%, 65%, 70%, 75%, or 80% more mutations in NF1, TERT, CDKN2a, NRAS, KRAS, HRAS, BRAF, KIT, PTEN, TP53, ARID1A, ARID1B, ARID2, or a combination thereof, compared to a normal biological sample; or at least 10%, 15%, 20%, 25%, 30%, 35%, 40%, 45%, 50%, 55%, 60%, 65%, 70%, 75%, or 80% more mutations in TERT, NRAS, BRAF, or a combination thereof, compared to a normal biological sample. In some embodiments, the method further comprises isolating microbial DNA and/or microbial RNA. In some embodiments, the microbial DNA and/or microbial RNA is isolated from a skin sample. In some embodiments, the microbial DNA and/or microbial RNA is isolated from the epidermis layer or from the dermis layer. In some embodiments, a sensitivity of the method is at least 95%. In some embodiments, a specificity of the method is at least 90%.

Disclosed herein, in certain embodiments, is a method of detecting nucleic acid expression level and modification in a skin sample, comprising: (a) obtaining a skin sample from an individual in need thereof using a plurality of adhesive patches; (b) contacting the skin sample with a plurality of beads; (c) co-isolating RNA and genomic DNA from the plurality of beads; (d) amplifying both the RNA and genomic DNA extracted from step (c); (e) detecting the expression level of a RNA of interest from the RNA of step (d); and (d) detecting a mutational change, a methylation status, or a combination thereof from a gene of interest from the genomic DNA of step (d).

Disclosed herein, in certain embodiments, is a method for detecting nucleic acid expression level and modification in a biological sample, comprising: (a) obtaining the biological sample from an individual in need thereof; and (b) detecting gene expression of HNC, PRAME, DNMT1, DNMT3A, DNMT3B, DNMT3L, KRT1, KRT10, IVL, TGase5, or a combination thereof, mutational change of NF1, TERT, CDKN2a, NRAS, KRAS, HRAS, BRAF, KIT, PTEN, TP53, ARID1A, ARID1B, ARID2, or a combination thereof, and/or methylation status of KRT10, KRT14, KRT15, KRT80, or a combination thereof in the biological sample. In some embodiments, the gene expression is of LINC, PRAME, DNMT1, DNMT3A, DNMT3B, DNMT3L, KRT1, KRT10, IVL, or TGase5. In some embodiments, the gene expression level is determined by contacting the biological sample with a set of probes that hybridizes to LINC, PRAME, DNMT1, DNMT3A, DNMT3B, DNMT3L, KRT1, KRT10, IVL, or TGase5, and detect binding between LINC, PRAME, DNMT1, DNMT3A, DNMT3B, DNMT3L, KRT1, KRT10, IVL, or TGase5 and the set of probes. In some embodiments, the gene expression is determined by contacting the biological sample with a set of probes that hybridizes to one and no more than ten genes selected from: LINC, PRAME, DNMT1, DNMT3A, DNMT3B, DNMT3L, KRT1, KRT10, IVL, or TGase5 and detect binding between LINC, PRAME, DNMT1, DNMT3A, DNMT3B, DNMT3L, KRT1, KRT10, IVL, or TGase5 and the set of probes. In some embodiments, the mutational change comprises: a mutation in NF1, TERT, CDKN2a, NRAS, KRAS, HRAS, BRAF, KIT, PTEN, TP53, ARID1A, ARID1B, or ARID2; a mutation in TERT, NRAS, or BRAF; a mutation in at least two genes selected from a list consisting of TERT, NRAS, and BRAF; a mutation in BRAF and a mutation in NRAS; a mutation in BRAF and a mutation in TERT; a mutation in NRAS and a mutation in TERT; or a mutation in TERT. In some embodiments, the methylation status is detected in KRT10, KRT14, KRT15, KRT80, or a combination thereof. In some embodiments, the expression level of LINC, PRAME, DNMT1, DNMT3A, DNMT3B, DNMT3L, KRT1, KRT10, IVL, TGase5, or a combination thereof is detected and the methylation status of KRT10, KRT14, KRT15, KRT80, or a combination thereof is detected.

Disclosed herein, in certain embodiments, is a method for diagnosing whether an individual is at risk of developing a cancer, comprising: (a) obtaining a biological sample from the individual; (b) detecting gene expression of LINC, PRAME, DNMT1, DNMT3A, DNMT3B, DNMT3L, KRT1, KRT10, IVL, TGase5, or a combination thereof, mutational change of NF1, TERT, CDKN2a, NRAS, KRAS, HRAS, BRAE, KIT, PTEN, TP53, ARID1A, ARID1B, ARID2, or a combination thereof, and/or methylation status of KRT10, KRT14, KRT15, KRT80, or a combination thereof in the biological sample; and (c) diagnosing the individual as having a risk factor for developing a cancer when the gene expression is elevated relative to a normal sample, a mutational change is detected, and/or the methylation is increased relative to a normal sample. In some embodiments, the biological sample is obtained using a plurality of adhesive patches. In some embodiments, gene expression is measured from RNA obtained from the skin sample. In some embodiments, mutational change is measured from DNA obtained from the skin sample. In some embodiments, the RNA and DNA are co-isolated using a plurality of beads. In some embodiments, the plurality of beads is a plurality of silica-coated beads. In some embodiments, the plurality of silica-coated beads is a plurality of silica-coated magnetic beads. In some embodiments, the biological sample is a skin sample suspicious for melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, or dermatofibrosarcoma protuberans. In some embodiments, the skin sample comprises keratinocytes, melanocytes, basal cells, T-cells, or dendritic cells. In some embodiments, the normal sample is a sample obtained from a healthy region of the same individual or is a sample obtained from a different healthy individual. In some embodiments, the RNA is mRNA or cell-free circulating RNA. In some embodiments, the DNA is cell-free circulating DNA. In some embodiments, the plurality of adhesive patches comprises at least 4 adhesive patches. In some embodiments, the biological sample is obtained by pooling the plurality of adhesive patches. In some embodiments, an effective amount of skin sample is removed by the plurality of adhesive patches. In some embodiments, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of RNA or DNA. In some embodiments, a yield of RNA or DNA obtained from the biological sample is at least about 200 picograms, at least about 500 picograms, at least about 750 picograms, at least about 1000 picograms, at least about 1500 picograms, or at least about 2000 picograms. In some embodiments, measuring gene expression comprises quantitative polymerase chain reaction (qPCR), RNA sequencing, or microarray analysis. In some embodiments, the gene expression is of HNC, PRAME, DNMT1, DNMT3A, DNMT3B, DNMT3L, KRT1, KRT10, IVL, or TGase5. In some embodiments, the gene expression level is determined by contacting the biological sample with a set of probes that hybridizes to LINC, PRAME, DNMT1, DNMT3A, DNMT3B, DNMT3L, KRT1, KRT10, IVL, or TGase5, and detect binding between LINC, PRAME, DNMT1, DNMT3A, DNMT3B, DNMT3L, KRT1, KRT10, IVL, or TGase5 and the set of probes. In some embodiments, the gene expression is determined by contacting the biological sample with a set of probes that hybridizes to one and no more than ten genes selected from: LINC, PRAME, DNMT1, DNMT3A, DNMT3B, DNMT3L, KRT1, KRT10, IVL, or TGase5 and detect binding between LINC, PRAME, DNMT1, DNMT3A, DNMT3B, DNMT3L, KRT1, KRT10, IVL, or TGase5 and the set of probes. In some embodiments, detecting mutational change in the DNA comprises allele specific polymerase chain reaction (PCR) or a sequencing reaction. In some embodiments, the mutational change comprises: a mutation in NF1, TERT, CDKN2a, NRAS, KRAS, HRAS, BRAF, KIT, PTEN, TP53, ARID1A, ARID1B, or ARID2; a mutation in TERT, NRAS, or BRAF; a mutation in at least two genes selected from a list consisting of TERT, NRAS, and BRAF; a mutation in BRAF and a mutation in NRAS; a mutation in BRAF and a mutation in TERT; a mutation in NRAS and a mutation in TERT; or a mutation in TERT. In some embodiments, the methylation status is detected in KRT10, KRT14, KRT15, KRT80, or a combination thereof. In some embodiments, the expression level of HNC, PRAME, DNMT1, DNMT3A, DNMT3B, DNMT3L, KRT1, KRT10, IVL, TGase5, or a combination thereof is detected and the methylation status of KRT10, KRT14, KRT15, KRT80, or a combination thereof is detected. In some embodiments, the mutational change comprises at least 1.5×, 2×, 3×, 4×, 5×, 6×, 7×, 8×, 9×, 10×, 11×, or 12× more mutations in NF1, TERT, CDKN2a, NRAS, KRAS, HRAS, BRAF, KIT, PTEN, TP53, ARID1A, ARID1B, ARID2, or a combination thereof, compared to a normal biological sample. In some embodiments, the mutational change comprises at least 10%, 15%, 20%, 25%, 30%, 35%, 40%, 45%, 50%, 55%, 60%, 65%, 70%, 75%, or 80% more mutations in NF1, TERT, CDKN2a, NRAS, KRAS, HRAS, BRAF, KIT, PTEN, TP53, ARID1A, ARID1B, ARID2, or a combination thereof, compared to a normal biological sample. In some embodiments, a sensitivity of the method is at least 95%. In some embodiments, a specificity of the method is at least 90%.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a graph comparing total RNA yield in picogram (y-axis) using different magnetic beads. Bars on graph represent repeat extraction and analysis from each group.

FIG. 2 depicts a graph of total RNA yield in picogram (y-axis) using magnetic beads in different lysis buffers. Bars on graph represent repeat extraction and analysis from each group.

FIG. 3 depicts a graph of total RNA yield in picogram (y-axis) using silica-coated magnetic beads and column extraction from a first experiment. Bars on graph represent repeat extraction and analysis from each group.

FIG. 4 depicts a graph of total RNA yield in picogram (y-axis) using optimized silica-coated magnetic beads and column extraction from a second experiment. Bars on graph represent repeat extraction and analysis from each group.

FIG. 5 depicts a graph of recovery of RNA extracted from samples (T0_Direct) with optimized silica-coated magnetic beads in 3 separate runs (Bead-KF 1, 2, 3). X-axis shows RNA in lysis buffer in picogram, and y-axis shows Cₜ values.

FIG. 6 depicts a graph of RNA extraction using silica-coated magnetic beads and column extraction from skin samples collected on adhesive patches. X-axis shows test subjects (from whom patches were collected), and y-axis shows Cₜ values.

FIG. 7 depicts a graph of RNA extraction using silica-coated magnetic beads and column extraction from skin samples collected on adhesive patches. RNA extraction is from 2 mm punches and 6 mm punches of the adhesive patches. X-axis shows size of punches, and y-axis shows Cₜ values.

FIG. 8 depicts a graph of RNA yield in picogram (y-axis) using silica-coated magnetic beads (batches 1-7) and column extraction (batch 8) from skin samples collected on adhesive patches. X-axis shows batch of sample extractions.

FIG. 9 depicts a graph of RNA yield distribution in picogram (y-axis) using silica-coated magnetic beads and column extraction from skin samples collected on adhesive patches. X-axis shows data from silica-coated magnetic beads (Silica Bead) and column extraction (PicoPure Column).

FIG. 10A depicts a gel electrophoresis of RNA extraction from skin samples collected on adhesive patches using silica-coated magnetic beads (Silica Bead) and column extraction (PicoPure Column).

FIG. 10B depicts a gel electrophoresis of RNA extraction from skin samples collected on adhesive patches using silica-coated magnetic beads with and without tRNA in lysis buffer.

FIG. 11A depicts a graph of total RNA quantification by qPCR in Silica Bead extraction from skin samples collected on adhesive patches. X-axis shows test subjects, and y-axis shows Cₜ values.

FIG. 11B depicts a graph of total genomic DNA quantification by qPCR from same Silica Bead extraction of same skin samples as that shown in FIG. 11A. X-axis shows test subjects, and y-axis shows Cₜ values.

FIG. 11C depicts a graph of total RNA yield in picogram (y-axis) from Silica Bead extraction as shown in FIG. 11A. X-axis shows test subjects.

FIG. 11D depicts a graph of total genomic DNA (gDNA) yield in picogram (y-axis) from same Silica Bead extraction as shown in FIG. 11B. X-axis shows test subjects.

FIG. 12A depicts a graph of total human RNA yield in picogram (log, y-axis) co-extracted using Silica Bead from skin samples collected non-invasively on adhesive patches from different body sites. X-axis shows sites of skin sample collection: forehead, inner arm, and hand.

FIG. 12B depicts a graph of total human genomic DNA (gDNA) yield in picogram (log, y-axis) co-extracted using Silica Bead from skin samples collected non-invasively on adhesive patches from different body sites. X-axis shows sites of skin sample collection: forehead, inner arm, and hand.

FIG. 12C depicts a graph of correlation of human RNA yield in picogram (log, x-axis) versus human genomic DNA yield in picogram (log, y-axis), in Silica Bead extractions from skin samples collected on adhesive patches.

FIG. 12D depicts a graph of total microbiome DNA yield in picogram (log, y-axis) co-extracted using Silica Bead from skin samples collected non-invasively using adhesive patches. X-axis shows sites of skin sample collection: forehead, inner arm, and hand.

FIG. 13 depicts a gel electrophoresis of polymerase chain reaction (PCR) products of different gene exomes amplified from genomic DNA extracted from skin samples collected on adhesive patches from a healthy test subject (control) as compared to a melanoma cell line.

FIG. 14 depicts an adhesive patch and procedure for non-invasive skin sample collection.

FIG. 15 depicts a graph of biomass of non-invasively obtained skin tissue samples from 5 anatomical areas. X-axis shows the 5 anatomical areas: mastoid, temple, forehead, chest, and abdomen. Y-axis shows skin tissue weight in milligram (mean weight on each adhesive patch).

FIG. 16 depicts a transmission electron microscopy analysis of skin tissue collected on adhesive patches.

FIG. 17 depicts a graph of comparison of total RNA yield in picogram (log, y-axis) from freshly harvested or stored patches. X-axis shows four storage conditions tested: 7 days at 25° C., 7 days at 40° C., 7 days at 60° C., and 10 days at −80° C.

FIG. 18A depicts a graph of threshold cycle (Cₜ) values (y-axis) of quantitative PCR analysis of genes from freshly harvested or stored patches. Conditions for stored patches include 7 days at 25° C. Target genes (x-axis) include actin, B2M, PPIA, and CMIP.

FIG. 18B depicts a graph of threshold cycle (Cₜ) values (y-axis) of quantitative PCR analysis of genes from freshly harvested or stored patches. Conditions for stored patches include 7 days at 40° C. Target genes (x-axis) include actin, B2M, PPIA, and CMIP.

FIG. 18C depicts a graph of threshold cycle (Cₜ) values (y-axis) of quantitative PCR analysis of genes from freshly harvested or stored patches. Conditions for stored patches include 7 days at 60° C. Target genes (x-axis) include actin, B2M, PPIA, and CMIP.

FIG. 18D depicts a graph of threshold cycle (Cₜ) values (y-axis) of quantitative PCR analysis of genes from freshly harvested or stored patches. Conditions for stored patches include 10 days at −80° C. Target genes (x-axis) include actin, B2M, PPIA, and CMIP.

FIG. 19A depicts a graph of total yield of human RNA in picogram (log, y-axis) from skin sample sites. X-axis shows skin sample sites including forehead, inner arm, and hand.

FIG. 19B depicts a graph of total yield of human genomic DNA (gDNA) in picogram (log, y-axis) from skin sample sites. X-axis shows skin sample sites including forehead, inner arm, and hand.

FIG. 19C depicts a graph of a correlation of total human RNA yield in picogram (log, x-axis) and total human genomic DNA (gDNA) yield in picogram (log, y-axis).

FIG. 19D depicts a graph of total yield of microbiome DNA in picogram (log, y-axis) from skin sample sites. X-axis shows skin sample sites including forehead, inner arm, and hand.

FIG. 20A depicts a gel electrophoresis of polymerase chain reaction amplification of human BRAF gene target exon from genomic DNA (gDNA) isolated using Silica Bead from skin samples collected on adhesive patches.

FIG. 20B depicts a chromatogram of human BRAF target exon sequence (SEQ ID NO: 14) from Sanger sequencing of a heterozygous mutated sample.

FIG. 21A depicts a graph of percentage of BRAF and NRAS mutations detected in PLA positive (PLA+) samples.

FIG. 21B depicts a chart of percentage BRAF, NRAS, BRAF or NRAS, and BRAF and NRAS mutations detected in PLA positive samples.

FIG. 22A depicts a graph of percentage of BRAF and NRAS mutations detected in PLA negative (PLA−) samples.

FIG. 22B depicts a chart of percentage of BRAF, NRAS, BRAF or NRAS, and BRAF and NRAS mutations detected in PLA negative samples.

FIG. 23 depicts a graph comparing percentage of mutations detected in PLA positive (PLA+) and PLA negative (PLA−) samples. Mutation detected is shown on an x-axis and includes: BRAF, NRAS, TERT, at least 1 gene mutant detected (“at least 1 mut”), 2 gene mutants detected (“2 any mut”), and 3 gene mutants detected (“all 3 mut”).

FIG. 24 illustrates the PCR detection of Streptococci (Strep), Staphylococci (Staph), Propionibacteria (PropiB), Corynebacteria (CoryneB) and Fungi from an adhesive patch collected epidermal skin sample.

FIG. 25A-25C illustrate the cell count obtained from each body site from human host skin (FIG. 25A), microbiome (FIG. 25B), and fungi (FIG. 25C).

FIG. 26A and FIG. 26B show the total microbiome counts determined using either the TaqMAN probe (FIG. 26A) or using the SYBR dye (FIG. 26B) for detection of the amplified product.

FIG. 27A-FIG. 27C show the analysis of Corynebacterium, Staphylococcus, and the total microbiome numbers in skin samples harvested from different body sites from 3 test subjects. FIG. 27A shows the total microbiome count. FIG. 27B shows the total count from Corynebacterium. FIG. 27C shows the total count from Staphylococcus.

FIG. 28A-FIG. 28C show the analysis of the changes in the numbers of fungi and microbiome in samples collected from the different layers of skin, using forehead site as an example, from 3 test subjects (3 bar colors). FIG. 28A shows the analysis of the total human skin cells per patch. FIG. 28B shows the total fungi per patch. FIG. 28C shows the total microbiome per patch.

FIG. 29A-FIG. 29C show the analysis of the changes of Corynebacterium and Staphylococcus numbers in skin samples collected from different layers of skin, using forehead site as an example FIG. 29A shows the total microbiome per patch. FIG. 29B shows the number of Corynebacterium cells per patch. FIG. 29C shows the number of Staphylococcus cells per patch.

FIG. 30A and 30B illustrate total bacteria collected (FIG. 30A) or total fungi collected (FIG. 30B) at different skin depth level. The X-axis indicates the 1^{st}, 2^{nd}, 3^{rd}, and 4^{th} sampling of the same skin area.

FIG. 31A-FIG. 31C show the analysis of the changes of Corynebacterium and Staphylococcus in percentage of total microbiome from the different layers of skin, using forehead site as an example, of the 3 test subjects. FIG. 31A illustrates the change in bacteria composition from the forehead region in Subject 1. FIG. 31B illustrates the change in bacteria composition from the forehead region in Subject 2. FIG. 31C illustrates the change in bacteria composition from the forehead region in Subject 3.

FIG. 32 depicts a gel electrophoresis of polymerase chain reaction (PCR) products of KRT10 and KRT14.

FIG. 33A shows results from a RNA recovery test.

FIG. 33B shows results from DNA and RNA extraction from skin samples collected on adhesive patch using the method described herein in comparison with an extraction method described by Zymol Research (Cat. D4100-2-3).

FIG. 34A illustrates an exemplary test design and procedure to determine the compatibility of the magnetic beads from Zymo Research with the extraction method described herein.

FIG. 34B illustrates total RNA and gDNA obtained from the tested eluents.

FIG. 35 illustrates gDNA and total RNA extraction utilizing a 100 μL DT MB:30 μL Zymo MB ratio compared to the control, which contains 100 μL of DT MB.

### DETAILED DESCRIPTION

Skin diseases and disorders are common human illnesses. In some instances, quality of a sample and availability of a sample are important factors for diagnosis and treatment of such diseases and disorders. There are several methods of obtaining tissue material including, for example, invasive techniques such as surgical biopsies.

In some cases, from a tissue material, either genomic DNA or RNA is extracted and isolated for subsequent analysis and diagnosis of disease. Diagnosis of disease using various methods such as gene expression analysis and histopathology, in some instances, has reduced specificity or sensitivity. Often these methods require invasive techniques.

Provided herein are methods and compositions for non-invasively obtaining a biological sample and improving sensitivity and specificity of downstream applications. In certain embodiments, detecting both expression level and mutational change of one or more genes in the biological sample results in improved sensitivity and specificity. In some instances, human and microbial genes are detected. By determining the expression level and mutational change of the one or more genes in the biological sample, information about a disease or disorder, in some instances, are obtained. In some embodiments, such information is used for diagnosing the disease or disorder.

Provided herein are methods and compositions for detecting expression level and mutational change from a biological sample. In some instances, the biological sample comprises a blood sample, saliva sample, urine sample, serum sample, plasma sample, tear sample, skin sample, tissue sample, hair sample, sample from cellular extracts, or a tissue biopsy sample. In some instances, the biological sample comprises a skin sample.

### Biological Samples and Methods of Use

Biological samples are obtained using a variety of methods. In some instances, obtaining a biological sample such as a skin sample comprises, but is not limited to, scraping of the skin, biopsy, suction, blowing and other techniques. In some instances, obtaining the biological sample is non-invasive. For example, the biological sample is obtained from a skin using a skin sample collector. In some cases, the biological sample is obtained by applying an adhesive patch to a skin sample in a manner sufficient to adhere a sample of the skin to the adhesive patch, and removing the adhesive patch from the skin in a manner sufficient to retain the adhered skin sample to the adhesive patch. In some instances, the patch comprises a rubber adhesive on a polyurethane film. In some instances, about one to about ten adhesive patches or one to ten applications of the patch are applied to and removed from the skin.

In some instances, an effective amount of skin sample is removed by the adhesive patch. In some instances, the effective amount comprises between about 50 micrograms to about 500 micrograms, between about 100 micrograms to about 450 micrograms, between about 100 micrograms to about 350 micrograms, between about 100 micrograms to about 300 micrograms, between about 120 micrograms to about 250 micrograms, or between about 150 micrograms to about 200 micrograms of nucleic acid material.

In some instances, the adhesive patch comprises various material. In some embodiments, the adhesive patch comprises a matrix comprising a synthetic rubber compound. In some embodiments, the adhesive patch does not comprise a latex material, a silicone material, or a combination thereof.

In some embodiments, the adhesive patch comprises a first central collection area having a skin facing surface comprising the adhesive matrix and a second area extending from the periphery of the first collection area creating a tab. In some cases, the first central collection area and the second area are comprised of different materials. In some cases, the first central collection area is comprised of a polyurethane carrier film.

In some embodiments, the skin sample is obtained from a site on a body. In some instances, the skin sample is obtained from a chest, forehead, hand, mastoid, temple, abdomen, arm, or leg. In some cases, the skin sample is not obtained from an area located on the palms, soles of feet, or mucous membranes.

In some embodiments, the skin sample is obtained from a skin lesion. In some cases, the skin lesion is a pigmented skin lesion comprising a mole, dark colored skin spot, or melanin containing skin area. In some cases, the skin lesion is an area on the skin surface that is suspicious for melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, Merkel cell carcinoma, sebaceous carcinoma, squamous cell carcinoma, and dermatofibrosarcoma protuberans. In some instances, the skin lesion is suspicious for skin cancer. Exemplary skin cancer include, but are not limited to, melanoma, basal cell carcinoma (BCC), squamous cell carcinoma (SCC), angiosarcoma, cutaneous B-cell lymphoma, cutaneous T-cell lymphoma, dermatofibrosarcoma protuberans, Merkel cell carcinoma, and sebaceous gland carcinoma. In some instances, the skin lesion is suspicious for melanoma.

In some cases, the skin lesion is from about 5 mm to about 20 mm in diameter.

Methods and compositions as described herein, in certain embodiments, result in obtaining various layers of skin. In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis.

In some instances, cells are obtained from the skin using methods and compositions as described herein. Exemplary cells that are obtained include, but are not limited to, keratinocytes, melanocytes, basal cells, T-cells, Merkel cells, Langerhans cells, fibroblasts, macrophages, adipocytes, and dendritic cells. In some cases, melanocytes, dendritic cells, and/or T cells are obtained from the skin using methods and compositions described herein. In some cases, cells such as melanocytes, dendritic cells, and/or T cells are obtained from the stratum corneum layer using methods and compositions described herein.

In additional instances, nucleic acids obtained from cells such as keratinocytes, melanocytes, basal cells, T-cells, Merkel cells, Langerhans cells, fibroblasts, macrophages, adipocytes, and dendritic cells and/or from microbiome from different skin layers are obtained simultaneously using methods and compositions described herein. In such cases, nucleic acids obtained from cells such as keratinocytes, melanocytes, basal cells, T-cells, Merkel cells, Langerhans cells, fibroblasts, macrophages, adipocytes, and dendritic cells and/or from microbiome from different epidermal layers are obtained simultaneously using methods and compositions described herein.

Provided herein are methods and compositions for extraction of nucleic acids from a biological sample such as a sample collected using an adhesive patch. In some instances, nucleic acids are extracted using any technique that does not interfere with subsequent analysis. In some instances, this technique uses alcohol precipitation using ethanol, methanol or isopropyl alcohol. In some instances, this technique uses phenol, chloroform, or any combination thereof. In some instances, this technique uses cesium chloride. In some instances, this technique uses sodium, potassium or ammonium acetate or any other salt commonly used to precipitate the nucleic acids.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

Nucleic acids isolated from a sample using methods and compositions described herein, in certain embodiments, are human or non-human. In some instances, the nucleic acids are human. For example, the sample comprises human RNA and human genomic DNA. In some instances, the sample further comprises non-human nucleic acids. In some instances, the non-human nucleic acids are microbial nucleic acids. In some instances, the microbial nucleic acids include, but are not limited to, pathogenic nucleic acids, bacterial nucleic acids, viral nucleic acids, fungal nucleic acids, parasitic nucleic acids, and any combination thereof.

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods and compositions for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um ,1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 500 nanograms, or higher.

In some cases, methods described herein provide less than less than 10%, less than 8%, less than 5%, less than 2%, less than 1%, or less than 0.5% product yield variations between samples.

In some cases, methods described herein provide a substantially homogenous population of a nucleic acid product.

In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods and compositions for detecting an expression level of one or more genes of interest from nucleic acids isolated from a biological sample. In some instances, the expression level is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the expression level is determined using PCR. In some instances, the expression level is determined using qPCR. In some instances, the expression level is determined using a microarray. In some instances, the expression level is determined by sequencing.

Provided herein are methods and compositions for detecting a mutational change of one or more genes of interest from nucleic acids isolated from a biological sample. In some instances, the mutational change is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the mutational change is detected using allele specific PCR. In some instances, the mutational change is detected using sequencing. In some instances, the sequencing is performed using the Sanger sequencing method. In some instances, the sequencing involves the use of chain terminating dideoxynucleotides. In some instances, the sequencing involves gel-electrophoresis. In some instances, the sequencing is performed using a next generation sequencing method. In some instances, sequencing includes, but not limited to, single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis.

### Illustrative Genes of Interest

Methods and compositions described herein are used for detecting at least one of expression level and mutational change of a gene of interest. In some instances, the gene of interest is implicated in a disease. In some instances, the disease is a skin disorder or disease. In some instances, the skin disorder or disease is a skin cancer. Exemplary genes associated with skin cancer and, in some instances, detected using methods described herein include, but are not limited to, AJUBA, AKT, ARID, BRAF, BRM, CASP8, CDKN1B, CDKN2, CDKN2A, DNMT1, DNMT3A, DNMT3B, DNMT3L, FAT1, HRAS, KIT, KMT2C, KMT2D, KRT1, KRT10, IVL, MC1R, MCV, NF1, NOTCH1, NRAS, PDGFRA, PIK3CA, PLCG1, PRKG1, PTCH1, PTCH2, PTEN, RB1, SMO, SUFU, TERT, TET2, TGase5, and XRCC3. In some instances, the gene of interest is NF1, TERT, CDKN2a, NRAS, KRAS, HRAS, BRAF, KIT, PTEN, TP53, ARID1A, ARID1B, or ARID2. In some instances, the gene of interest is BRAF. In some instances, the gene of interest is NRAS. In some instances, the gene of interest is TERT. In some instances, the gene of interest is LINC. In some instances, the gene of interest is of preferentially expressed antigen in melanoma (PRAME). In some instances, the genes of interest comprise DNMT1, DNMT3A, DNMT3B, DNMT3L, KRT1, KRT10, IVL, or TGase 5. In some instances, the gene of interest is DNMT1, DNMT3A, DNMT3B, or DNMT3L. In some instances, the gene of interest is KRT1 or KRT10. In some instances, the gene of interest is IVL. In some instances, the gene of interest is TGase5. In some instances, expression level of the gene interest is determined using a gene expression assay. An exemplary gene expression assays includes a pigmented lesion assay.

In some embodiments, a mutational change comprises one or more mutations in AJUBA, AKT, ARID, BRAF, BRM, CASP8, CDKN1B, CDKN2, CDKN2A, DNMT3A, FAT1, HRAS, KIT, KMT2C, KMT2D, MC1R, MCV, NF1, NOTCH1, NRAS, PDGFRA, PIK3CA, PLCG1, PRKG1, PTCH1, PTCH2, PTEN, RB1, SMO, SUFU, TERT, TET2, or XRCC3. In some instances, the mutational change comprises one or more mutations in BRAF, NRAS, TERT, or a combination thereof. In some cases, the mutational change comprises a mutation in BRAF and a mutation in NRAS. In some cases, the mutational change comprises a mutation in NRAS and a mutation in TERT. In some cases, the mutational change comprises a mutation in BRAF and a mutation in TERT. In some cases, the mutational change comprises a mutation in BRAF. In some cases, the mutational change comprises a mutation in NRAS. In additional cases, the mutational change comprises a mutation in TERT.

The gene PRAME encodes an antigen that is preferentially expressed in human melanomas and that is recognized by cytolytic T lymphocytes. The encoded protein is involved in growth of cancer cells. In some instances, over expression of PRAME is correlated with skin cancer. In some instances, over expression of PRAME is correlated with melanoma.

The gene LINC, also known as Long Intergenic Non-protein Coding refers to, in some instances, LINC00518 or C6orf218. In some instances, over expression of HNC is correlated with skin cancer. In some instances, over expression of HNC is correlated with melanoma.

The gene BRAF, also known as B-Raf proto-oncogene, serine/threonine kinase, V-Raf murine sarcoma viral oncogene homolog B1, and RAFB1, encodes the B-Raf protein. The B-Raf protein is involved in cell signaling and cell growth. In some instances, a mutation in BRAF is correlated with a skin cancer. Exemplary mutations in BRAF which translate to amino acid positions in the B-Raf protein include, but are not limited to, G466, G469, V600, and K601, wherein the amino acids correspond to positions 466, 469, 600, and 601 of SEQ ID NO: 1. In some cases, the mutations include V600E, V600K, K601E, G469A, and G466V.

The gene NRAS, also known as neuroblastoma RAS viral oncogene homolog, NRAS proto-oncogene, encodes the NRAS protein. The NRAS protein is involved in cell division, cell differentiation, and apoptosis. In some instances, a mutation in NRAS is correlated with a skin cancer. Exemplary mutations in NRAS, which translate to amino acid positions in the NRAS protein include, but are not limited to, Q61 and G12, wherein the amino acids correspond to positions 61 and 12 of SEQ ID NO: 2. In some instances, the mutations include Q61K, Q61R, G12A, and G12P.

TERT, also known as Telomerase Reverse Transcriptase or Telomerase-Associated Protein 2, encodes the TERT protein. The TERT protein is the catalytic subunit of the protein telomerase. In some instances, a mutation in TERT is correlated with a skin cancer. In some instances, a mutation is in the TERT promoter. In some instances, a mutation is at least or about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more than 200 base pairs upstream of the translation start site of the TERT promoter. In some instances, one or more mutations is in the TERT promoter. In some instances, the one or more mutations in the TERT promoter is a G to A mutation. In some instances, the one or more mutations in the TERT promoter is a T to G mutation. In some instances, the one or more mutations in the TERT promoter is a C to T mutation. In some instances, one or more mutations in the TERT promoter result in increased expression of TERT. In some instances, one or more mutations in the TERT promoter result in increased expression or activity of TERT protein. Exemplary mutations in TERT include, but not limited to, 1,295,228 C>T (C228T) and 1,295,250 C>T (C250T). In some instances, C228T is a mutation corresponding to −124 C>T from the translation start site in the TERT promoter. In some instances, C250T is a mutation corresponding to −146 C>T from the translation start site in the TERT promoter.

The gene CDKN2A, also known as cyclin-dependent kinase inhibitor 2A, encodes two proteins p16^{INK4a} and p14^{ARF}. p16^{INK4a} and p14^{ARF} are involved in cellular senescence. In some instances, a mutation in CDKN2A is correlated with skin cancer. In some instances, mutations in CDKN2A comprise deletions and mutations throughout the coding region.

DNA methyltransferases (DNMTs) such as (DNMT1, DNMT3A, DNMT3B, and DNMT3L) catalyze de novo methylation of unmethylated cytosine. In some instances, DNMT1 is expressed in the hair follicle and in the basal layer of the epidermis. Sometimes, its expression diminishes upon differentiation. DNMT1 copies the pattern of methyl marks from the parent strand to the daughter strand after cell division. In some cases, DNMT1 up-regulates growth genes but down-regulates differential genes (e.g., inhibits differentiation). Knockdown of DNMT1 leads to a premature epidermal differentiation and hypoplasia. In some instances, DNMT3A and DNMT3B are expressed in the basal level of the epidermis. In some cases, DNMT3A and DNMT3B play a role in establishing DNA methylation in nonepidermal genes during skin stem cell differentiation (>20% of the repressed genes are methylated de novo during epidermal differentiation. In some instances, overexpression of DNMT1, DNMT3A, DNMT3B, and/or DNMT3L is associated with a skin cancer. In some cases, overexpression of DNMT1, DNMT3A, DNMT3B, and/or DNMT3L is associated with cutaneous squamous cell carcinoma (cSCC). In additional cases, overexpression of DNMT1, DNMT3A, DNMT3B, and/or DNMT3L is associated with actinic keratosis (AK).

Keratin family members 1 and 10 are expressed in the spinous and granular layers of the epidermis. In some instances, overexpression of keratin 1 gene KRT1 and/or keratin 10 gene KRT10 is associated with a skin cancer. In some cases, overexpression of KRT1 and/or KRT10 is associated with cutaneous squamous cell carcinoma (cSCC). In additional cases, overexpression of KRT1 and/or KRT10 is associated with actinic keratosis (AK).

Involucrin is a protein component of the skin and is encoded by the IVL gene. In some instances, overexpression of IVL is associated with a skin cancer. In some cases, overexpression of IVL is associated with cutaneous squamous cell carcinoma (cSCC). In additional cases, overexpression of IVL is associated with actinic keratosis (AK).

Transglutaminase 5 protein catalyzes the formation of protein crosslinks between glutamine and lysine residues and is encoded by the TGase5 gene. In some instances, overexpression of TGase5 is associated with a skin cancer. In some cases, overexpression of TGase5 is associated with cutaneous squamous cell carcinoma (cSCC). In additional cases, overexpression of TGase5 is associated with actinic keratosis (AK).

In some instances, a mutational change is in a gene implicated in melanoma. In some instances, the mutational change results in changes in expression of the gene. In some instances, a mutational change results in changes in expression or activation of encoded protein. In some instances, changes in the expression or the activation of the encoded protein comprise a decrease in the expression or the activation. In some instances, changes in the expression or the activation of the encoded protein comprise an increase in the expression or the activation. For example, the mutational change in the gene results in constitutive activation of the encoded protein. In some instances, a mutational change in the gene results in increased expression of encoded protein. In some instances, the gene is AJUBA, AKT, ARID, BRAF, BRM, CASP8, CDKN1B, CDKN2, CDKN2A, DNMT3A, FAT1, HRAS, KIT, KMT2C, KMT2D, MC1R, MCV, NF1, NOTCH1, NRAS, PDGFRA, PIK3CA, PLCG1, PRKG1, PTCH1, PTCH2, PTEN, RB1, SMO, SUFU, TERT, TET2, XRCC3, DNMT1, DNMT3A, DNMT3B, DNMT3L, KRT1, KRT10, IVL, or TGase5. In some instances, the gene is NF1, TERT, CDKN2a, NRAS, KRAS, HRAS, BRAF, KIT, PTEN, TP53, ARID1A, ARID1B, or ARID2. In some instances, the gene is BRAF. Exemplary mutations in BRAF which translate to amino acid positions in the B-Raf protein include, but are not limited to, V600E, V600K, K601E, G469A, and G466V. In some instances, the gene is NRAS. Exemplary mutations in NRAS which translate to amino acid positions in the NRAS protein include, but are not limited to, Q61K, Q61R, G12A, and G12P. In some instances, the gene is TERT.

In some instances, one or more genes of interest from isolated nucleic acids are analyzed. In some instances, from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 5 to about 100, from about 5 to about 80, from about 5 to about 60, from about 5 to about 40, from about 5 to about 20, from about 10 to about 100, from about 10 to about 80, from about 10 to about 60, from about 10 to about 40, from about 20 to about 80, from about 20 to about 60, from about 20 to about 40, from about 30 to about 80, from about 30 to about 60, from about 40 to about 60, from about 2 to about 10, from about 2 to about 8, or from about 2 to about 6 genes of interest from the isolated nucleic acids are analyzed. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the genes of interest include, but are not limited to, AJUBA, AKT, ARID, BRAF, BRM, CASP8, CDKN1B, CDKN2, CDKN2A, DNMT3A, FAT1, HRAS, KIT, KMT2C, KMT2D, MC1R, MCV, NF1, NOTCH1, NRAS, PDGFRA, PIK3CA, PLCG1, PRKG1, PTCH1, PTCH2, PTEN, RB1, SMO, SUFU, TERT, TET2, XRCC3, PRAME, and LINC. In some instances, an expression level is determined in the one or more of the genes of interest. In some instances, a mutational change is determined in the one or more of the genes of interest.

In some instances, an expression level and a mutational change are both determined in one or more genes of interest. In some instances, the expression level is detected using a gene expression assay such as a pigmented lesion assay. In some instances, the gene expression assay detects the expression of at least one of PRAME and LINC. In some instances, the mutational change in one or more genes of interest is determined following a pigmented lesion assay. For example, a biological sample is determined to be positive or negative whenever the gene expression of either PRAME or LINC is detected while the same gene's expression is not detected in healthy cells. In some instances, a biological sample is determined to be positive whenever the gene expression of either PRAME or LINC is at least or about 5%, 10%, 15%, 20%, 25%, 30%, 35%, 40%, 45%, 50%, 55%, 60%, 65%, 70%, 75%, 80%, 85%, 90%, 95%, or more than 95% increased as compared to expression of PRAME or LINC in healthy cells. In some instances, a biological sample is determined to be positive whenever the gene expression of either PRAME or LINC is at least or about 1.5-fold, 2-fold, 2.5-fold, 3-fold, 3.5-fold, 4-fold, 5-fold, or more than 5-fold higher as compared to expression of PRAME or LINC in healthy cells. In some instances, one or more mutations in the gene of interest are also detected. The genes of interest include, but are not limited to, AJUBA, AKT, ARID, BRAF, BRM, CASP8, CDKN1B, CDKN2, CDKN2A, DNMT3A, FAT1, HRAS, KIT, KMT2C, KMT2D, MC1R, MCV, NF-1, NOTCH1, NRAS, PDGFRA, PIK3CA, PLCG1, PRKG1, PTCH1, PTCH2, PTEN, RB1, SMO, SUFU, TERT, TET2, and XRCC3. In some instances, the gene of interest is at least one of BRAF, NRAS, and TERT. In some instances, the one or more mutation is in BRAF. In some instances, the one or more mutation is in NRAS. In some instances, the one or more mutation is in TERT. In some instances, the one or more mutation is in any two genes from BRAF, NRAS, and TERT. In some instances, the one or more mutation is all three genes BRAF, NRAS, and TERT.

In some instances, one or more mutations in the gene of interest are more prevalent in a biological sample positive for a pigmented lesion compared to a biological sample negative for a pigmented lesion. For example, one or more mutations in the gene of interest is at least or about 1.5×, 2×, 3×, 4×, 5×, 6×, 7×, 8×, 9×, 10×, 11×, 12×, or more than 12× more prevalent in a biological sample positive for a pigmented lesion compared to a biological sample negative for a pigmented lesion. In some instances, one or more mutations in the gene of interest is at least or about 10%, 15%, 20%, 25%, 30%, 35%, 40%, 45%, 50%, 55%, 60%, 65%, 70%, 75%, 80%, or more than 80% prevalent in a biological sample positive for a pigmented lesion compared to a biological sample negative for a pigmented lesion.

Expression level or mutational change once detected, in certain embodiments, provides information regarding a disease in an individual. In some instances, both expression level and mutational change provide information regarding the disease in the individual. Information regarding the disease includes, but is not limited to, identification of a disease state, likelihood of treatment success for a given disease state, identification of progression of a disease state, and identification of a disease stage. In some instances, at least one of expression level and mutational change are compared to a control sample for identification of the disease state, determining likelihood of treatment success for the given disease state, identification of progression of the disease state, or identification of the disease stage. In some instances, the control sample is any sample that is used for making any one of these determinations. In some instances, the control sample is from a healthy individual. In some instances, the control is a sample from an individual with a known disease or disorder. In some instances, the control is from a database or reference. In some instances, the control is a normal sample from the same individual. In some instances, the normal sample is a sample that does not comprise cancer, disease, or disorder, or a sample that would test negative for cancer, disease, or disorder. In some instances, the normal sample is assayed at the same time or at a different time.

In some instances, an expression level of one or more genes of interest from a biological sample varies as compared to a control sample. In some instances, the expression level is at least or about 5%, 6%, 7%, 8%, 9%, 10%, 11%, 12%, 13%, 14%, 15%, 16%, 17%, 18%, 19%, 20%, 22%, 24%, 28%, 30%, 35%, 40%, 45%, 50%, 55%, 60%, 65%, 70%, 75%, 80%, 85%, 90%, 95% or more than 95% increased as compared to control. In some instances, the expression level is at least or about 5%, 6%, 7%, 8%, 9%, 10%, 11%, 12%, 13%, 14%, 15%, 16%, 17%, 18%, 19%, 20%, 22%, 24%, 28%, 30%, 35%, 40%, 45%, 50%, 55%, 60%, 65%, 70%, 75%, 80%, 85%, 90%, 95% or more than 95% decreased as compared to control. In some instances, the expression level is increased or decreased in a range of about 1% to about 100%, about 10% to about 90%, about 20% to about 80%, about 30% to about 70%, or about 40% to about 60%.

In some instances, a mutational change in one or more genes of interest from a biological sample comprises at least one mutation as compared to a control sample. In some instances, the one or more genes of interest from the biological sample comprises at least or about 1, 2, 3, 4, 5, 6, 7, 8, 9, 10, or more than 10 mutations.

In some instances, at least one of expression level and mutational change of a gene of interest provide information regarding melanoma. For example, the at least one of expression level and mutational change of a gene of interest provide information regarding a stage of melanoma. In some instances, the at least one of expression level and mutational change of a gene of interest is associated with a stage of melanoma. Characteristics of the stages of melanoma include, but are not limited to, benign lesion, intermediate lesion, melanoma in situ, invasive melanoma, and metastasis. In some instances, one or more mutations in a gene of interest indicate a risk factor for melanoma or the stage of melanoma. In some instances, the gene of interest is AJUBA, AKT, ARID, BRAF, BRM, CASP8, CDKN1B, CDKN2, CDKN2A, DNMT3A, FAT1, HRAS, KIT, KMT2C, KMT2D, MC1R, MCV, NF-1, NOTCH1, NRAS, PDGFRA, PIK3CA, PLCG1, PRKG1, PTCH1, PTCH2, PTEN, RB1, SMO, SUFU, TERT, TET2, or XRCC3. In some instances, the gene of interest is at least one of BRAF, NRAS, and TERT.

Methods and compositions provided herein comprising detecting expression level and mutational change result in improved sensitivity and specificity for diagnosis or prognosis of disease. In some instances, detecting expression level and mutational change result in improved sensitivity and specificity for diagnosis or prognosis of melanoma. In some instances, sensitivity is improved by at least or about 55%, 60%, 65%, 70%, 75%, 80%, 85%, 90%, 95% or more than 95% as compared to other diagnosis or prognosis methods. In some instances, specificity is improved by at least or about 55%, 60%, 65%, 70%, 75%, 80%, 85%, 90%, 95% or more than 95% as compared to other diagnosis or prognosis methods. The other diagnosis or prognosis methods include, but are not limited to, morphology histopathology, pattern histopathology, and RNA only based gene expression assays.

A method of selecting an individual at risk for developing a skin condition for treatment, comprising (a) obtaining a skin sample comprising a lesion from the individual; (b) analyzing the skin sample to detect the expression level of PRAME, LINC, or a combination thereof, and the presence of a mutation in BRAF, NRAS, TERT, or a combination thereof; and (c) determining that the individual is at risk for developing a skin condition if the expression level of PRAME, LINC, or a combination thereof is at least 2-fold or higher relative to the expression level of PRAME, LINC, or a combination thereof of a normal skin sample; and the presence of a mutation in BRAF, NRAS, TERT, or a combination thereof. In some instances, the individual is at risk for developing a skin condition if the expression level of PRAME, LINC, or a combination thereof is at least 2-fold or higher relative to the expression level of PRAME, LINC, or a combination thereof of a normal skin sample, and the presence of a mutation in BRAF and a mutation in NRAS. In some instances, the individual is at risk for developing a skin condition if the expression level of PRAME, LINC, or a combination thereof is at least 2-fold or higher relative to the expression level of PRAME, LINC, or a combination thereof of a normal skin sample, and the presence of a mutation in BRAF and a mutation in TERT. In some instances, the individual is at risk for developing a skin condition if the expression level of PRAME, LINC, or a combination thereof is at least 2-fold or higher relative to the expression level of PRAME, LINC, or a combination thereof of a normal skin sample, and the presence of a mutation in TERT and a mutation in NRAS. In some instances, the individual is at risk for developing a skin condition if the expression level of PRAME, LINC, or a combination thereof is at least 2-fold or higher relative to the expression level of PRAME, LINC, or a combination thereof of a normal skin sample, and the presence of a mutation in TERT. In some instances, the expression level of PRAME, LINC, or a combination is at least 3-fold, 4-fold, 5-fold, 6-fold, 7-fold, 8-fold, 9-fold, 10-fold, or higher relative to the expression level of PRAME, LINC, or a combination in a normal skin sample. In some instances, a mutation in BRAF correlates to amino acid residue G466, G469, V600, or K601 of the encoded B-Raf protein, in which the amino acids correspond to positions 466, 469, 600, and 601 of SEQ ID NO: 1. In some instances, the mutation in BRAF correlates to V600E, V600K, K601E, G469A, or G466V. In some instances, a mutation in NRAS correlates to amino acid residue G12 or Q61 of the encoded NRAS protein, in which the amino acids correspond to positions 12 and 61 of SEQ ID NO: 2. In some instances, the mutation in NRAS correlates to G12A, G12P, Q61K, or Q61R.

### Microbiome Profile

Methods and compositions for detecting expression level and mutational change in a biological sample, in certain embodiments, comprise detecting a microbiome profile. In some instances, detecting the microbiome profile is used for diagnosis or prognosis of a disease or disorder.

In some instances, the microbiome comprises microbial material including, but not limited to, bacteria, archaea, protists, fungi, and viruses. In some instances, the microbial material comprises a gram-negative bacterium. In other instances, the microbial material comprises a gram-positive bacterium. In some cases, the microbial material comprises Proteobacteria, Actinobacteria, Bacteriodetes, and/or Firmicutes.

Non-limiting examples of bacteria include bacteria from the genus Actinomycetales, Anaerococcus, Bacillales, Bifidobacterium, Enhydrobacter, Finegoldia, Carnobacterium, Coryneobacterium, Lactobacillus, Lactococcus, Leunconostoc, Macrooccus, Micrococcineae, Oenococcus, Pediococcus, Peptoniphilus, Propionibacterium, Salinicoccus, Sphingomonas, Staphylococcus, Strepococcus, Tetragenoccus, and Weissella.

In some instances, the microbiome comprises microbial material from fungal species. In some cases, the microbial material comprises a fungus from the genus Malassezia (or Pityrosporum), Aspergillus, Candida, Cryptococcus, Rhodotorula, and/or Epicoccum. Exemplary fungi include, but are not limited to, Candida tropicalis, Candida parapsilosis, Candida orthopsilosis, Cryptococcus flavus, Cryptococcus dimennae, Cryptococcus diffluens, Aspergillus fumigatus, and Pityrosporum ovale.

In some instances, the microbiome comprises microbial material from Archaean species. In some cases, the microbial material comprises an archaean from phyla Thaumarchaeota and/or Euryarchaeota.

In some instances, the microbiome comprises microbial material from a virus. In some cases, the microbial material comprises viruses from the family Polyomoaviridae, Papillomaviridae, and/or Circoviridae. In some cases, the microbial material comprises one or more viruses such as human alpha, beta, and/or gamma papillomaviruses.

In some instances, the microbiome comprises microbial material from protist. In some cases, the microbial material comprises a protist pathogen.

In some instances, microbial nucleic acids are extracted using methods and compositions previously described. For example, microbial nucleic acids are collected on a skin site using an adhesive patch. Exemplary skin sites for collecting microbial nucleic acids include, but are not limited to, chest, forehead, hand, mastoid, temple, abdomen, arm, or leg. In some instances, the microbial nucleic acids are characteristic of a skin site.

In some instances, microbial nucleic acids are further isolated and purified using silica-coated magnetic beads. In some instances, microbial nucleic acids are amplified. In some instances, microbial nucleic acids are subject to sequencing. In some instances, the microbial nucleic acids comprise RNA or DNA.

Methods and compositions described herein, in certain embodiments, comprise co-analyzing microbial nucleic acids and human nucleic acids from a same sample. In some instances, microbial nucleic acids are distinguished from human nucleic acids by detecting expression levels of genes present in microbes but not in humans For example, 16S ribosomal RNA gene is used.

Detection of microbial nucleic acids, in certain embodiments, is used for diagnosing or prognosing a disease or disorder. In some instances, the disease or disorder is a skin disease or skin disorder. Exemplary skin diseases or disorders that, in certain embodiments, are associated with skin microbiome include, but are not limited to, psoriasis, atopic dermatitis, seborrhoeic dermatitis, and acne. In some instances, the disease or disorder is skin cancer.

In some instances, detecting microbial nucleic acids improves sensitivity and specificity for diagnosis or prognosis of a disease or disorder, particularly when used in combination with detecting expression level and mutational change of one or more genes of interest in human RNA and human DNA. In some instances, sensitivity is improved by at least or about 55%, 60%, 65%, 70%, 75%, 80%, 85%, 90%, 95% or more than 95% as compared to other diagnosis or prognosis methods. In some instances, specificity is improved by at least or about 55%, 60%, 65%, 70%, 75%, 80%, 85%, 90%, 95% or more than 95% as compared to other diagnosis or prognosis methods.

### CpG Methylation Profiling

DNA methylation is the attachment of a methyl group at the C5-position of the nucleotide base cytosine and the N6-position of adenine. Methylation of adenine primarily occurs in prokaryotes, while methylation of cytosine occurs in both prokaryotes and eukaryotes. In some instances, methylation of cytosine occurs in the CpG dinucleotides motif. In other instances, cytosine methylation occurs in, for example CHG and CHH motifs, where H is adenine, cytosine or thymine. In some instances, one or more CpG dinucleotide motif or CpG site forms a CpG island, a short DNA sequence rich in CpG dinucleotide. In some instances, a CpG island is present in the 5′ region of about one half of all human genes. CpG islands are typically, but not always, between about 0.2 to about 1 kb in length. Cytosine methylation further comprises 5-methylcytosine (5-mCyt) and 5-hydroxymethylcytosine.

The CpG (cytosine-phosphate-guanine) or CG motif refers to regions of a DNA molecule where a cytosine nucleotide occurs next to a guanine nucleotide in the linear strand. In some instances, a cytosine in a CpG dinucleotide is methylated to form 5-methylcytosine. In some instances, a cytosine in a CpG dinucleotide is methylated to form 5-hydroxymethylcytosine.

In some embodiments, a gene of interest is differentially methylated in a skin cancer when compared to normal skin. In such cases, the CpG methylation status of a gene of interest is determined utilizing a method described herein. In some instances, the methylation status of keratin 10 gene KRT10, keratin 14 gene KRT14, keratin 15 gene KRT15, and/or keratin 80 gene KRT80 is determined utilizing a method described herein, e.g., utilizing a biological sample processing method described herein to obtain a genomic DNA sample and subsequent methylation analysis to determine the methylation status of the gene.

In some instances, methylation analysis is carried out by any means known in the art. A variety of methylation analysis procedures are known in the art and may be used. These assays allow for determination of the methylation state of one or a plurality of CpG sites within a biological sample. In addition, these methods may be used for absolute or relative quantification of methylated nucleic acids. Such methylation assays involve, among other techniques, two major steps. The first step is a methylation specific reaction or separation, such as (i) bisulfate treatment, (ii) methylation specific binding, or (iii) methylation specific restriction enzymes. The second major step involves (i) amplification and detection, or (ii) direct detection, by a variety of methods such as (a) PCR (sequence-specific amplification) such as Taqman(R), (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

In an embodiment, the methylation status of a gene of interest is determined using a Sanger sequencing or a Next-Generation sequencing (NGS) method. Suitable next generation sequencing technologies include the 454 Life Sciences platform (Roche, Branford, Conn.) (Margulies et al. 2005 Nature, 437, 376-380); lllumina's Genome Analyzer, GoldenGate Methylation Assay, or Infinium Methylation Assays, i.e., Infinium HumanMethylation 27K BeadArray or VeraCode GoldenGate methylation array (Illumina, San Diego, Calif.; Bibkova et al, 2006, Genome Res. 16, 383-393; U.S. Pat. Nos. 6,306,597 and 7,598,035 (Macevicz); U.S. Pat. No. 7,232,656 (Balasubramanian et al.)); QX200™ Droplet Digital™ PCR System from Bio-Rad; or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al); the Helicos True Single Molecule DNA sequencing technology (Harris et al, 2008 Science, 320, 106-109; U.S. Pat. Nos. 7,037,687 and 7,645,596 (Williams et al); U.S. Pat. No. 7,169,560 (Lapidus et al); U.S. Pat. No. 7,769,400 (Harris)), the single molecule, real-time (SMRT™) technology of Pacific Biosciences, and sequencing (Soni and Meller, 2007, Clin. Chem. 53, 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). These systems allow the sequencing of many nucleic acid molecules isolated from a specimen at high orders of multiplexing in a parallel fashion. Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

In an embodiment, the methylation status of a gene of interest is determined using methylation-Specific PCR (MSP). MSP allows for assessing the methylation status of one or more CpG sites, independent of the use of methylation-sensitive restriction enzymes (Herman et al, 1996, Proc. Nat. Acad. Sci. USA, 93, 9821-9826; U.S. Pat. Nos. 5,786,146, 6,017,704, 6,200,756, 6,265,171 (Herman and Baylin); U.S. Pat. Pub. No. 2010/0144836 (Van Engeland et al)). Briefly, DNA is modified by a deaminating agent such as sodium bisulfite to convert unmethylated, but not methylated cytosines to uracil, and subsequently amplified with primers specific for methylated versus unmethylated DNA. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes. The ColoSure™ test is a commercially available test for colon cancer based on the MSP technology and measurement of methylation of the vimentin gene (Itzkowitz et al, 2007, Clin Gastroenterol. Hepatol. 5(1), 111-117). Alternatively, one may use quantitative multiplexed methylation specific PCR (QM-PCR), as described by Fackler et al. Fackler et al, 2004, Cancer Res. 64(13) 4442-4452; or Fackler et al, 2006, Clin. Cancer Res. 12(11 Pt 1) 3306-3310.

In some instances, the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534) is utilized for determining the methylation status of a gene of interest. COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al, 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from micro-dissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfo nation buffer; DNA recovery reagents or kits (e.g., precipitation, ultrafiltration, affinity column); desulfonation buffer; and DNA recovery components.

In an embodiment, the methylation profile of selected CpG sites is determined using MethyLight and/or Heavy Methyl Methods. The MethyLight and Heavy Methyl assays are a high-throughput quantitative methylation assay that utilizes fluorescence-based real-time PCR (Taq Man(R)) technology that requires no further manipulations after the PCR step (Eads, C. A. et al, 2000, Nucleic Acid Res. 28, e 32; Cottrell et al, 2007, J. Urology 177, 1753, U.S. Pat. No. 6,331,393 (Laird et al)). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. In some cases, sequence discrimination occurs either at the level of the amplification process or at the level of the fluorescence detection process, or both. In some cases, the MethyLight assay is used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites. Typical reagents (e.g., as might be found in a typical MethyLight-based kit) for MethyLight analysis may include, but are not limited to: PCR primers for specific gene (or methylation-altered DNA sequence or CpG island); TaqMan(R) probes; optimized PCR buffers and deoxynucleotides; and Taq polymerase. The MethyLight technology is used for the commercially available tests for lung cancer (epi proLung BL Reflex Assay); colon cancer (epi proColon assay and mSEPT9 assay) (Epigenomics, Berlin, Germany) PCT Pub. No. WO 2003/064701 (Schweikhardt and Sledziewski).

Quantitative MethyLight uses bisulfite to convert genomic DNA and the methylated sites are amplified using PCR with methylation independent primers. Detection probes specific for the methylated and unmethylated sites with two different fluorophores provides simultaneous quantitative measurement of the methylation. The Heavy Methyl technique begins with bisulfate conversion of DNA. Next specific blockers prevent the amplification of unmethylated DNA. Methylated genomic DNA does not bind the blockers and their sequences will be amplified. The amplified sequences are detected with a methylation specific probe. (Cottrell et al, 2004, Nuc. Acids Res. 32:e10).

The Ms-SNuPE technique is a quantitative method for assessing methylation differences at specific CpG sites based on bisulfite treatment of DNA, followed by single-nucleotide primer extension (Gonzalgo and Jones, 1997, Nucleic Acids Res. 25, 2529-2531). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. In some cases, small amounts of DNA are analyzed (e.g., micro-dissected pathology sections), and the method avoids utilization of restriction enzymes for determining the methylation status at CpG sites. Typical reagents (e.g., as is found in a typical Ms-SNuPE-based kit) for Ms-SNuPE analysis include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In another embodiment, the methylation status of selected CpG sites is determined using differential Binding-based Methylation Detection Methods. For identification of differentially methylated regions, one approach is to capture methylated DNA. This approach uses a protein, in which the methyl binding domain of MBD2 is fused to the Fc fragment of an antibody (MBD-FC) (Gebhard et al, 2006, Cancer Res. 66:6118-6128; and PCT Pub. No. WO 2006/056480 A2 (Relhi)). This fusion protein has several advantages over conventional methylation specific antibodies. The MBD FC has a higher affinity to methylated DNA and it binds double stranded DNA. Most importantly the two proteins differ in the way they bind DNA. Methylation specific antibodies bind DNA stochastically, which means that only a binary answer can be obtained. The methyl binding domain of MBD-FC, on the other hand, binds DNA molecules regardless of their methylation status. The strength of this protein—DNA interaction is defined by the level of DNA methylation. After binding genomic DNA, eluate solutions of increasing salt concentrations can be used to fractionate non-methylated and methylated DNA allowing for a more controlled separation (Gebhard et al, 2006, Nucleic Acids Res. 34: e82). Consequently this method, called Methyl-CpG immunoprecipitation (MCIP), not only enriches, but also fractionates genomic DNA according to methylation level, which is particularly helpful when the unmethylated DNA fraction should be investigated as well.

In an alternative embodiment, a 5-methyl cytidine antibody to bind and precipitate methylated DNA. Antibodies are available from Abeam (Cambridge, MA), Diagenode (Sparta, NJ) or Eurogentec (c/o AnaSpec, Fremont, Calif.). Once the methylated fragments have been separated they may be sequenced using microarray based techniques such as methylated CpG-island recovery assay (MIRA) or methylated DNA immunoprecipitation (MeDIP) (Pelizzola et al, 2008, Genome Res. 18, 1652-1659; 0′ Geen et al, 2006, BioTechniques 41(5), 577-580, Weber et al, 2005, Nat. Genet. 37, 853-862; Horak and Snyder, 2002, Methods Enzymol, 350, 469-83; Lieb, 2003, Methods Mol Biol, 224, 99-109). Another technique is methyl-CpG binding domain column/segregation of partly melted molecules (MBD/SPM, Shiraishi et al, 1999, Proc. Natl. Acad. Sci. USA 96(6):2913-2918).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. Pat. Nos. 7,910,296; 7,901,880; and No. 7,459,274. In some embodiments, amplification can be performed using primers that are gene specific.

For example, there are methyl-sensitive enzymes that preferentially or substantially cleave or digest at their DNA recognition sequence if it is non-methylated. Thus, an unmethylated DNA sample is cut into smaller fragments than a methylated DNA sample Similarly, a hypermethylated DNA sample is not cleaved. In contrast, there are methyl-sensitive enzymes that cleave at their DNA recognition sequence only if it is methylated. Methyl-sensitive enzymes that digest unmethylated DNA suitable for use in methods of the technology include, but are not limited to, Hpall, Hhal, Maell, BstUI and Acil. In some instances, an enzyme that is used is Hpall that cuts only the unmethylated sequence CCGG. In other instances, another enzyme that is used is Hhal that cuts only the unmethylated sequence GCGC. Both enzymes are available from New England BioLabs(R), Inc. Combinations of two or more methyl-sensitive enzymes that digest only unmethylated DNA are also used. Suitable enzymes that digest only methylated DNA include, but are not limited to, Dpnl, which only cuts at fully methylated 5′-GATC sequences, and McrBC, an endonuclease, which cuts DNA containing modified cytosines (5-methylcytosine or 5-hydroxymethylcytosine or N4-methylcytosine) and cuts at recognition site 5′ . . . PumC(N4o-3ooo) PumC . . . 3′ (New England BioLabs, Inc., Beverly, Mass). Cleavage methods and procedures for selected restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Sambrook et al. (See Sambrook et al. Molecular Biology: A Laboratory Approach, Cold Spring Harbor, N.Y. 1989) provide a general description of methods for using restriction enzymes and other enzymes.

In some instances, a methylation-dependent restriction enzyme is a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., Dpnl) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC, McrA, MrrA, Bisl, Glal and Dpnl. One of skill in the art will appreciate that any methylation-dependent restriction enzyme, including homologs and orthologs of the restriction enzymes described herein, is also suitable for use in the present invention.

In some cases, a methylation-sensitive restriction enzyme is a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al, 22(17) NUCLEIC ACIDS RES. 3640-59 (1994). Suitable methylation-sensitive restriction enzymes that do not cleave DNA at or near their recognition sequence when a cytosine within the recognition sequence is methylated at position C5 include, e.g., Aat II, Aci I, Acd I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapAl I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N6 include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

In alternative embodiments, adaptors are optionally added to the ends of the randomly fragmented DNA, the DNA is then digested with a methylation-dependent or methylation-sensitive restriction enzyme, and intact DNA is subsequently amplified using primers that hybridize to the adaptor sequences. In this case, a second step is performed to determine the presence, absence or quantity of a particular gene in an amplified pool of DNA. In some embodiments, the DNA is amplified using real-time, quantitative PCR.

In other embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

In some instances, the quantity of methylation of a locus of DNA is determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. Pat. No. 7,910,296.

The methylated CpG island amplification (MCA) technique is a method that can be used to screen for altered methylation patterns in genomic DNA, and to isolate specific sequences associated with these changes (Toyota et al, 1999, Cancer Res. 59, 2307-2312, U.S. Pat. No. 7,700,324 (Issa et al)). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

Additional methylation detection methods include those methods described in, e.g., U.S. Pat. Nos. 7,553,627; 6,331,393; U.S. patent Ser. No. 12/476,981; U.S. Patent Publication No. 2005/0069879; Rein, et al, 26(10) NUCLEIC ACIDS RES. 2255-64 (1998); and Olek et al, 17(3) NAT. GENET. 275-6 (1997).

In another embodiment, the methylation status of selected CpG sites is determined using Methylation-Sensitive High Resolution Melting (HRM). Recently, Wojdacz et al. reported methylation-sensitive high resolution melting as a technique to assess methylation. (Wojdacz and Dobrovic, 2007, Nuc. Acids Res. 35(6) e41; Wojdacz et al. 2008, Nat. Prot. 3(12) 1903-1908; Balic et al, 2009 J. Mol. Diagn. 11 102-108; and US Pat. Pub. No. 2009/0155791 (Wojdacz et al)). A variety of commercially available real time PCR machines have HRM systems including the Roche LightCycler480, Corbett Research RotorGene6000, and the Applied Biosystems 7500. HRM may also be combined with other amplification techniques such as pyrosequencing as described by Candiloro et al. (Candiloro et al, 2011, Epigenetics 6(4) 500-507).

In another embodiment, the methylation status of selected CpG locus is determined using a primer extension assay, including an optimized PCR amplification reaction that produces amplified targets for analysis using mass spectrometry. The assay can also be done in multiplex. Mass spectrometry is a particularly effective method for the detection of polynucleotides associated with the differentially methylated regulatory elements. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. This method is described in detail in PCT Pub. No. WO 2005/012578A1 (Beaulieu et al). For methylation analysis, the assay can be adopted to detect bisulfite introduced methylation dependent C to T sequence changes. These methods are particularly useful for performing multiplexed amplification reactions and multiplexed primer extension reactions (e.g., multiplexed homogeneous primer mass extension (hME) assays) in a single well to further increase the throughput and reduce the cost per reaction for primer extension reactions.

Other methods for DNA methylation analysis include restriction landmark genomic scanning (RLGS, Costello et al, 2002, Meth. Mol Biol, 200, 53-70), methylation-sensitive-representational difference analysis (MS-RDA, Ushijima and Yamashita, 2009, Methods Mol Biol 507, 1 17-130). Comprehensive high-throughput arrays for relative methylation (CHARM) techniques are described in WO 2009/021141 (Feinberg and Irizarry). The Roche(R) NimbleGen(R) microarrays including the Chromatin Immunoprecipitation-on-chip (Ch1P-chip) or methylated DNA immunoprecipitation-on-chip (MeDIP-chip). These tools have been used for a variety of cancer applications including melanoma, liver cancer and lung cancer (Koga et al, 2009, Genome Res., 19, 1462-1470; Acevedo et al, 2008, Cancer Res., 68, 2641-2651; Rauch et al, 2008, Proc. Nat. Acad. Sci. USA, 105, 252-257). Others have reported bisulfate conversion, padlock probe hybridization, circularization, amplification and next generation or multiplexed sequencing for high throughput detection of methylation (Deng et al, 2009, Nat. Biotechnol 27, 353-360; Ball et al, 2009, Nat. Biotechnol 27, 361-368; U.S. Pat. No. 7,611,869 (Fan)). As an alternative to bisulfate oxidation, Bayeyt et al. have reported selective oxidants that oxidize 5-methylcytosine, without reacting with thymidine, which are followed by PCR or pyro sequencing (WO 2009/049916 (Bayeyt et al).

In some instances, quantitative amplification methods (e.g., quantitative PCR or quantitative linear amplification) are used to quantify the amount of intact DNA within a locus flanked by amplification primers following restriction digestion. Methods of quantitative amplification are disclosed in, e.g., U.S. Pat. Nos. 6, 180,349; 6,033,854; and 5,972,602, as well as in, e.g., DeGraves, et al, 34(1) BIOTECHNIQUES 106-15 (2003); Deiman B, et al., 20(2) MOL. BIOTECHNOL. 163-79 (2002); and Gibson et al, 6 GENOME RESEARCH 995-1001 (1996).

### Components of the Skin Collection Kit

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

### Tissue Sampling and Cellular Material

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids, proteins, lipids, and/or sugars. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities.

In still further or additional embodiments, the adhered skin sample comprises cellular material including nucleic acids such as RNA or DNA, or a polypeptide such as a protein, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 micrograms to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, or a polypeptide such as a protein, comprises an amount that is from about 50 micrograms to about 500 micrograms, from about 100 micrograms to about 450 micrograms, from about 100 micrograms to about 350 micrograms, from about 100 micrograms to about 300 micrograms, from about 120 micrograms to about 250 micrograms, from about 150 micrograms to about 200 micrograms, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as melanoma. In some embodiments, a software program or module scores a sample based on gene expression levels. In some embodiments, the sample score is compared with a reference sample score to determine if there is a statistical significance between the gene expression signature and a disease.

### Computer Program

The methods, software, media, and systems disclosed herein comprise at least one computer processor, or use of the same. In some instances, the computer processor comprises a computer program. In some instances, a computer program includes a sequence of instructions, executable in the digital processing device's CPU, written to perform a specified task. In some instances, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program, in some embodiments, are written in various versions of various languages.

The functionality of the computer readable instructions, in certain embodiments, are combined or distributed as desired in various environments. In some instances, a computer program comprises one sequence of instructions. In some instances, a computer program comprises a plurality of sequences of instructions. In some instances, a computer program is provided from one location. In some instances, a computer program is provided from a plurality of locations. In some instances, a computer program includes one or more software modules. In some instances, a computer program includes, in part or in whole, one or more web applications, one or more mobile applications, one or more standalone applications, one or more web browser plug-ins, extensions, add-ins, or add-ons, or combinations thereof.

### Web Application

In some instances, a computer program includes a web application. In light of the disclosure provided herein, those of skill in the art will recognize that a web application, in certain embodiments, utilizes one or more software frameworks and one or more database systems. In some instances, a web application is created upon a software framework such as Microsoft® .NET or Ruby on Rails (RoR). In some instances, a web application utilizes one or more database systems including, by way of non-limiting examples, relational, non-relational, feature oriented, associative, and XML database systems. Suitable relational database systems includes, by way of non-limiting examples, Microsoft® SQL Server, mySQL™, and Oracle®. Those of skill in the art will also recognize that a web application, in certain embodiments, is written in one or more versions of one or more languages. In some instances, a web application is written in one or more markup languages, presentation definition languages, client-side scripting languages, server-side coding languages, database query languages, or combinations thereof. In some instances, a web application is written to some extent in a markup language such as Hypertext Markup Language (HTML), Extensible Hypertext Markup Language (XHTML), or eXtensible Markup Language (XML). In some instances, a web application is written to some extent in a presentation definition language such as Cascading Style Sheets (CSS). In some instances, a web application is written to some extent in a client-side scripting language such as Asynchronous Javascript and XML (AJAX), Flash® Actionscript, Javascript, or Silverlight®. In some instances, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some instances, a web application is written to some extent in a database query language such as Structured Query Language (SQL). In some instances, a web application integrates enterprise server products such as IBM® Lotus Domino®. In some instances, a web application includes a media player element. In some instances, a media player element utilizes one or more of many suitable multimedia technologies including, by way of non-limiting examples, Adobe® Flash HTML 5, Apple® QuickTime®, Microsoft® Silverlight®, Java™, and Unity®.

Mobile Application

In some instances, a computer program includes a mobile application provided to a mobile digital processing device. In some instances, the mobile application is provided to a mobile digital processing device at the time it is manufactured. In some instances, the mobile application is provided to a mobile digital processing device via the computer network described herein.

In some instances, the mobile application is created by techniques known to those of skill in the art using hardware, languages, and development environments known to the art. Those of skill in the art will recognize that mobile applications, in certain embodiments, are written in several languages. Suitable programming languages include, by way of non-limiting examples, C, C++, C#, Featureive-C, Java™ Javascript, Pascal, Feature Pascal, Python™, Ruby, VB.NET, WML, and XHTML/HTML with or without CSS, or combinations thereof.

Suitable mobile application development environments, in some instances, are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. In some instances, other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some instances, a computer program includes a standalone application, which is a program that is run as an independent computer process, not an add-on to an existing process, e.g., not a plug-in. Those of skill in the art will recognize that standalone applications are often compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation are often performed, at least in part, to create an executable program. In some instances, a computer program includes one or more executable complied applications.

Web Browser Plug-In

In some instances, a computer program includes a web browser plug-in. In computing, a plug-in, in some instances, is one or more software components that add specific functionality to a larger software application. In some instances, makers of software applications support plug-ins to enable third-party developers to create abilities which extend an application, to support easily adding new features, and to reduce the size of an application. In some instances, when supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some instances, the toolbar comprises one or more web browser extensions, add-ins, or add-ons. In some instances, the toolbar comprises one or more explorer bars, tool bands, or desk bands.

In view of the disclosure provided herein, those of skill in the art will recognize that several plug-in frameworks, in some instances, are available that enable development of plug-ins in various programming languages, including, by way of non-limiting examples, C++, Delphi, Java™, PHP, Python™ and VB .NET, or combinations thereof.

In some instances, web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox , Google® Chrome, Apple® Safari®, Opera Software® Opera®, and KDE Konqueror. In some instances, web browser is a mobile web browser. In some instances, the mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules, in certain embodiments, are created by techniques known to those of skill in the art using machines, software, and languages known to the art. The software modules disclosed herein, in certain embodiments, are implemented in a multitude of ways. In some instances, a software module comprises a file, a section of code, a programming feature, a programming structure, or combinations thereof. In some instances, a software module comprises a plurality of files, a plurality of sections of code, a plurality of programming features, a plurality of programming structures, or combinations thereof. In some instances, the one or more software modules comprises, by way of non-limiting examples, a web application, a mobile application, and a standalone application. In some instances, software modules are in one computer program or application. In some instances, software modules are in more than one computer program or application. In some instances, software modules are hosted on one machine. In some instances, software modules are hosted on more than one machine. In some instances, software modules are hosted on cloud computing platforms. In some instances, software modules are hosted on one or more machines in one location. In some instances, software modules are hosted on one or more machines in more than one location.

### Databases

The medium, method, and system disclosed herein comprise one or more databases, or use of the same. In view of the disclosure provided herein, those of skill in the art will recognize that many databases, in certain embodiments, are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some instances, a database is internet-based. In some instances, a database is web-based. In some instances, a database is cloud computing-based. In some instances, a database is based on one or more local computer storage devices.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “individual(s)”, “subject(s)” and “patient(s)” mean any mammal In some embodiments, the mammal is a human In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “room temperature” encompasses a temperature of from about 22° C. to about 28° C. or from about 24° C. to about 26° C. In some instances, the term encompasses a temperature of about 22° C., about 23° C., about 24° C., about 25° C., about 26° C., about 27° C., or about 28° C.

A “normal” biological sample, e.g., a “normal” skin sample, corresponds to a sample which is used for comparative purposes. In some instances, a sample is “normal” in the sense that it does not exhibit any indications of, or is not believed to have, any disease or condition that would affect gene expression, mutational change, and/or methylation, for which it is to be used as the normal standard. In some cases, it will be appreciated that different stages of a cancer, e.g., a skin cancer, may be compared and in such cases, the “normal” sample may correspond to the earlier stage of cancer.

As used herein, a biological sample refers to any material obtained from an organism, e.g., human or non-human animal under investigation, which contains cells and includes, tissues body fluid or body waste.

A “site” or “CpG site” corresponds to a single site, which may be a single base position or a group of correlated base positions, e.g., a CpG site.

A “locus” corresponds to a region that includes multiple CpG sites. In some instances, a locus includes one CpG site.

A “CpG island” corresponds to a short DNA sequence comprising one or more CpG sites. In some instances, a CpG island comprises a region of at least 200-bp of DNA with a G+C content of at least 50% and observed CpG/expected CpG ratio of at least 0.6. In some instances, the CpG island has a GC content of about 55% to about 80%. In some cases, the CpG island comprises about 60% GC to about 70% GC. In some cases, moderately GC-rich CpG islands comprise about 50-60% GC. In some cases, extremely GC-rich CpG islands comprise greater than about 70% GC.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Reagent Preparation and RNA Extraction

Bulk Solution Preparation

A bulk solution was prepared according to Table 1. Reagents listed in Table 1 were added in the order listed to a 500 mL sterile bottle. The sterile bottle was capped and shaken to mix the reagents. 19 mL of the bulk solution (Solution A) was aliquoted to 50 mL conical tubes until all the solution was distributed. Each tube was labeled with the batch lot number and date and stored at room temperature in a dry, clean area.

Solution C Preparation

Solution C was prepared according to Table 2. Each reagent was added in the order listed in Table 2 to a 500 mL sterile bottle. The sterile bottle was capped and shaken to mix the reagents. 15 mL of the Solution C was aliquoted to 50 mL conical tubes until all the solution was distributed. Each tube was labeled with the batch lot number and date and stored at room temperature in a dry, clean area.

TABLE 1
Solution A Mixture

| | Final | Volume Used |
|---|---|---|
| Reagent | Concentration | (mL) |
| 6 M Guanidinium | 5 M | 416.7 |
| Thiocyanate | | |
| 1.0 M Tris-HCl (pH 7.5) | 10 mM | 5.00 |
| Nuclease-free water | | 78.3 |
| Total Volume | | 500 |

TABLE 2
Solution C Mixture

| | Final | Volume Used |
|---|---|---|
| Reagent | Concentration | (mL) |
| Potassium Chloride | 330 mM | 82.5 |
| (KCl), 2 M Solution | | |
| 1.0 M Tris-HCl (pH 7.5) | 67 mM | 33.5 |
| Nuclease-free water | | 384 |
| Total Volume | | 500 |

RNA Extraction

Shallow 2.0 mL cryofreeze aliquot tubes were obtained and placed in a microfuge tube rack. One tube was used for each patch. All tubes were labeled with the sample ID.

Using a sterile surgical blade or laser cut instrument, the demarcated lesion from each of the 4 patches was excised.

Lysis Buffer was prepared according to Table 1 with the addition of Proteinase K.

A wash buffer was prepared by adding 35 mL of pure Ethyl Alcohol (200 proof) to the tube of Solution C from above. The tube was caped and shaken to mix well. See Table 3.

TABLE 3
Wash Buffer 1

| | Final | Volume/rxn |
|---|---|---|
| Reagent | Concentration | (mL) |
| Solution C | 30% | 15 |
| Ethyl Alcohol, Pure (200 proof) | 70% | 35 |
| Total Volume | | 50 |

Preparation of Sample Lysis from Adhesive Patches

A Multipipette Repeater Stream was used to dispense 360 uL of the above Lysis Buffer solution into each lysis tube. Excised biopsy punches from the sample patches were transferred using sterile forceps to its corresponding lysis tube. The patch punches were placed in the lysis tube with adhesive side facing away from the tube wall.

The tubes were capped and rotated in a circular motion to evenly distribute the Master Mix throughout the patch in the tube. The tubes were then placed caps inward on horizontal shakers set at 3500 rpm and shaken for 30 minutes at room temperature.

Preparation of the KingFisher 96-Deep Well Plate

A KingFisher 96-deep well plate was unpacked and a clean KingFisher tip comb was placed in row A of the plate. The Ocean NanoTech Silica Bead stock tube from 4° C. was allowed to come to room temperature. The stock tube was vortexed to ensure the beads were well suspended in solution prior to use. 500 uL of the Wash Buffer was aliquoted to the wells of the KingFisher 96-deep well plate that were to receive sample. Following 30 minute sample lysis incubation on shakers, the sample lysis tubes were pulsed spin to collect lysate at the bottom of the tube. Sample lysate was transferred to the KingFisher 96-deep well plate. 20 uL of Ocean NanoTech Silica Beads was added to the wells.

Preparation of the KingFisher Elution Strips

Two KingFisher elution strips were placed on the white elution strip plate and labeled. 20 μL of nuclease-free water was pipetted to each well for both elution strips.

Total RNA Extraction on the KingFisher Duo Prime Instrument

The DTI_Protocol_4 Step Binding protocol was selected on the instrument. A Run Name was created. The sample-loaded 96-deep well plate was placed into the instrument. The elution strip holder was lifted and E1 elution strip was placed in the instrument sitting flush with the elution block. The elution strip holder was closed. The elution strip E2 was then loaded on the elution strip holder.

After both the 96-deep well plate and elution strips E1 and E2 were loaded to the instrument and locked down at their designated spots, the front lid of the instrument was closed. The samples were then processed.

Elution Combination and Storage of Remaining RNA

When the extraction process was completed, the elution strips E1 and E2 and the 96-deep well plate were removed. Elution 2 was combined with Elution 1 by carefully transferring the volume from the wells in elution strip E2 into the corresponding well of elution strip E1. Samples were mixed by slowly pipetting up and down several times. The total volume in the elution strip was 40 uL.

The eluent was stored in the elution strip in a −80° C. freezer or used for total RNA quantification by qPCR.

### Example 2. Comparison of Magnetic Beads with Different Surface Chemistry

RNA yield using magnetic beads of different surface chemistry was compared.

“Bead 1” comprised Sera-Mag speedbeads carboxylate modified magnetic particles that were 1 uM in diameter (ThermoFisher Scientific). “Bead 2” comprised carboxylate-magnetic particles that were 1 uM in diameter (Alpha BioBead Mag Bead, Ocean NanoTechnology). “Bead 3” comprised silica-coated magnetic beads that were 1 uM in diameter (Alpha BioBead Silica Bead, Ocean NanoTechnology). “Bead 4” comprised magnetic beads that were 1 uM in diameter (Machery Nagel B-Bead, Macherey-Nagel GmbH & Co. KG).

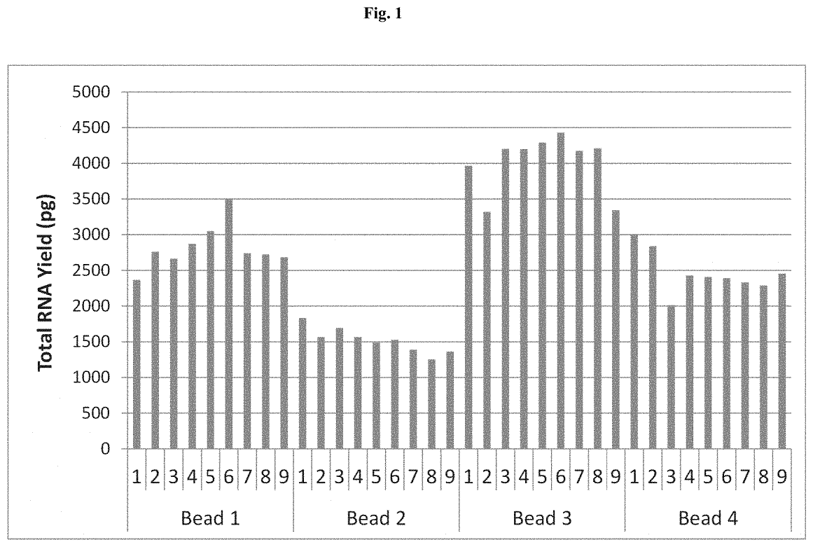

Referring to FIG. 1, there was improved total RNA yield in picogram (y-axis) using Bead 3.

This example shows silica-coated magnetic beads result in improved RNA yield.

### Example 3. Lysis Buffers for RNA Extraction

RNA yield was determined using silica-coated magnetic beads in different lysis buffers.

“Method 1” comprised using a first lysis buffer and Sera-Mag speedbeads carboxylate modified magnetic particles that were 1 uM in diameter (ThermoFisher Scientific). “Method 2” comprised using the first lysis buffer and silica-coated magnetic beads that were 1 uM in diameter (Alpha BioBead Silica Bead, Ocean NanoTechnology). “Method 3” comprised using a second lysis buffer and silica-coated magnetic beads that were 1 uM in diameter (Alpha BioBead Silica Bead, Ocean NanoTechnology).

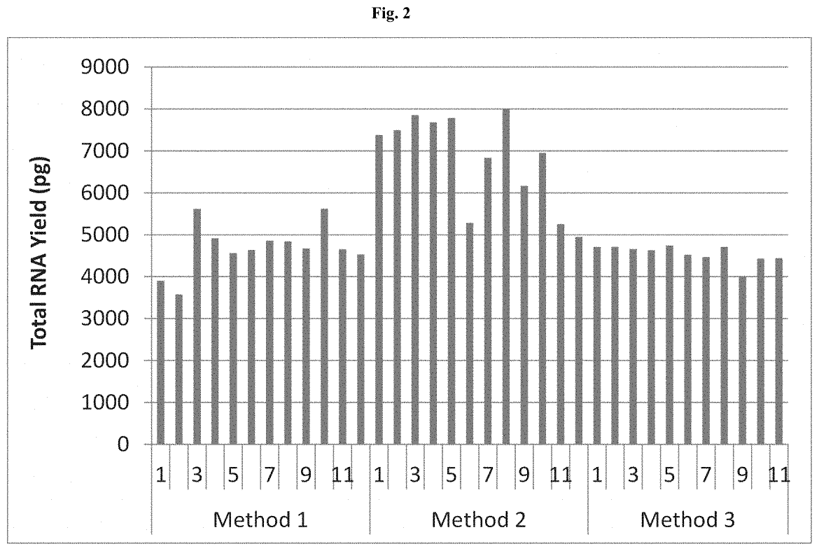

Referring to FIG. 2, there was increased total RNA yield in picogram (y-axis) using Method 2.

### Example 4. A First Formula for Silica-Coated Magnetic Beads

RNA yield using a first formula for silica-coated magnetic beads was compared to RNA yield using column extraction.

Adhesive patches were collected from 2 test subjects. Each patch was cut in half. One half of the patch was used for silica-coated magnetic bead extraction and the other half of the patch was used for column extraction using the PicoPure system. Each extraction was tested in triplicate by quantitative PCR (qPCR).

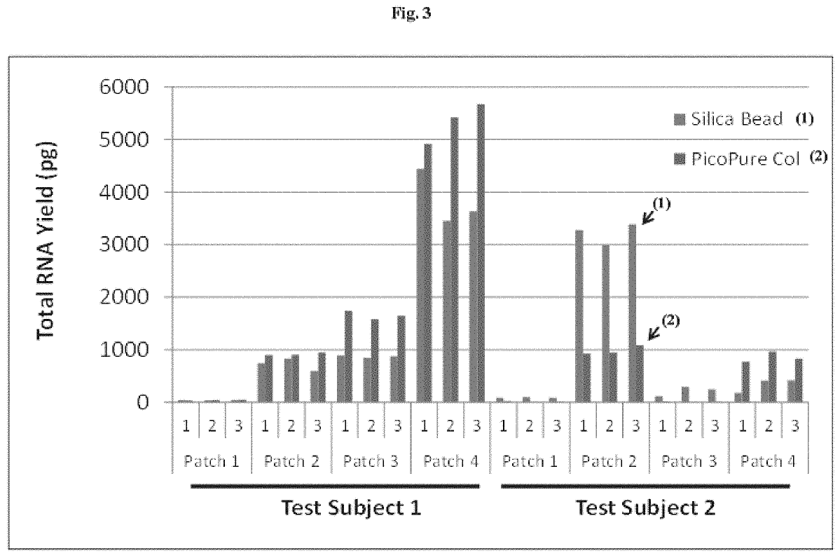

Referring to FIG. 3, the first formula for silica-coated magnetic beads (“Silica Bead,” gray bars (1), first bar on the left of a pair) produced similar total RNA yields in picogram (y-axis) to the column extraction (“PicoPure Col,” dark gray bars (2), second bar on the right of a pair).

This example shows silica-coated magnetic beads using the first formula result in extraction of RNA.

### Example 5. A Second Formula for Silica-coated Magnetic Beads

RNA yield using a second formula for silica-coated magnetic beads was compared to RNA yield using column extraction.

Adhesive patches were collected from multiple subjects. Each patch was cut in half. One half of the patch was used for silica-coated magnetic bead extraction and the other half of the patch was used for column extraction using the PicoPure system. The extraction using the silica-coated magnetic beads was compared to column extraction using Kingfisher™ Duo Prime Purification System (ThermoFisher Scientific) and qPCR.

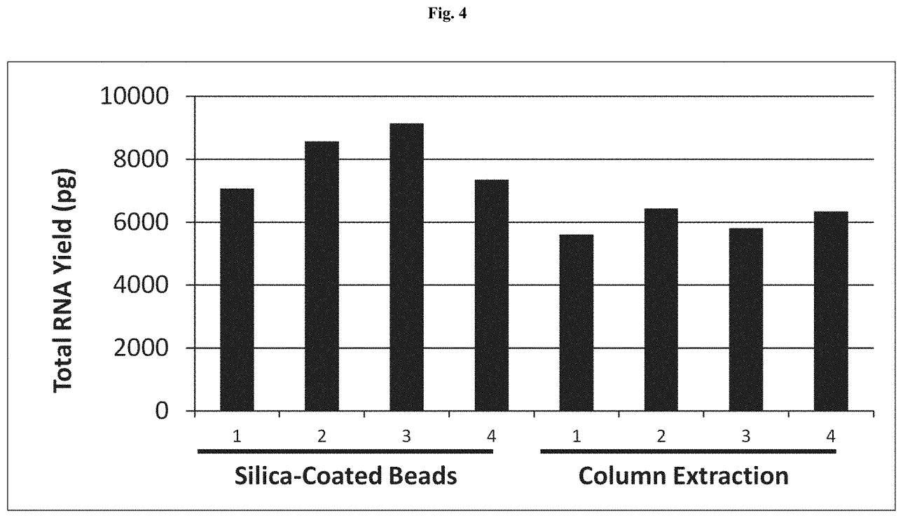

Referring to FIG. 4, the method using silica-coated magnetic beads and the second formula showed improved total RNA yield in picogram (y-axis) as compared to the column extraction.

This example shows silica-coated magnetic beads using the second formula for extraction result in improved RNA yield.

### Example 6. RNA Recovery Using Silica-Coated Magnetic Beads

Percentage of RNA recovered using silica-coated magnetic beads was determined.

Serial dilutions (0.6˜625 picogram) of Universal Human RNA (UHR) were spiked to lysis buffer. RNA was then recovered using the AccuBead™ magnetic beads on KingFisher™ Duo Prime Purification System (ThermoFisher Scientific) or used directly for qPCR.

“T0_Direct” refers to 2 uL of UHR spiked directly to RT-qPCR to measure the total amount of RNA, without going through the magnetic bead extraction. “Bead-KF(1),” “Bead-KF(2),” and “Bead-KF(3),” refer to 3 replicates of silica-coated magnetic bead extraction of lysis buffer spiked with 2 uL of the same UHR analyzed in “T0_Direct”. The magnetic bead recovered UHR are also analyzed in the same RT-qPCR as for the “T0_Direct” UHR samples to calculate percentage of recovery. Percentage recovery was determined by the following equation:

$$\% Recovery = \frac{RNA Yield ( bead \text{-} KF )}{Spike RNA ( T 0 \text{-} Direct )}$$

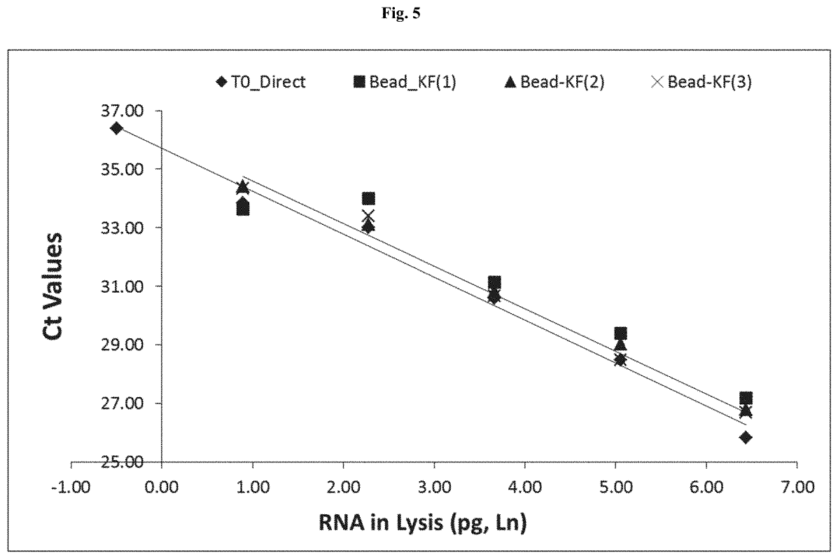

An average of about 71% of the total RNA spiked to the lysis buffer (71%, 69%, and 74% from the 3 replicates) was recovered using magnetic beads. Ln (RNA) was measured and is shown on the x-axis of FIG. 5 and Table 4 below.

TABLE 4

| | RNA (pg) Spiked | |
|---|---|---|
| | to Lysis Buff | Ln (RNA) |
| S2 | 625 | 6.44 |
| S3 | 156.25 | 5.05 |
| S4 | 39.06 | 3.67 |
| S5 | 9.77 | 2.28 |
| S6 | 2.44 | 0.89 |
| S7 | 0.61 | −0.49 |

This example shows silica-coated magnetic beads resulted in increased RNA recovered.

### Example 7. RNA Extraction from Skin Samples Collected on Adhesive Patches

RNA was collected from skin samples collected on full adhesive patches. Yield of RNA extracted using silica-coated magnetic beads was then compared to yield of RNA extracted using column extraction.

Skin samples were collected on full adhesive patches and used for RNA extraction. Skin samples were collected from the forehead of four test subjects. Patches from each test subject were randomly split for use with the KingFisher™ Duo Prime Purification System (ThermoFisher Scientific) or the PicoPure Column Two replicate extractions from each test subject were performed using the KingFisher™ Duo Prime Purification System and the PicoPure Column. qPCR was then performed.

Referring to FIG. 6, threshold cycle (Cₜ) values of RNA (y-axis) was compared between RNA extracted using KingFisher™ Duo Prime Purification System (“Bead-KF,” horizontal hashed bars) and RNA extracted using the PicoPure Column (“PicoPure Col,” black bars). Skin samples were collected on adhesive patches from 2 body sites (1, 2) of 4 test subjects (A, B, C, and D). Each patch was cut into 2 equal halves. One half was used for Bead-KF extraction, and the other half used for PicoPure Column extraction. Comparison of the Cₜ values showed a similar total RNA yield using KingFisher™ Duo Prime Purification System and the PicoPure Column.

This example shows RNA extraction from skin samples collected on adhesive patches using silica-coated magnetic beads.

### Example 8. RNA Extraction from Skin Samples Collected on Adhesive Patches

RNA was extracted from 6 mm and 2 mm punches of adhesive patches used to collect skin samples. Yield of RNA extracted using silica-coated magnetic beads was then compared to yield of RNA extracted using column extraction.

Adhesive patches were collected from the forehead skin of 2 test subjects. Six mm and 2 mm punches were made from each adhesive patch. Punches of each size (6 mm or 2 mm) were mixed and randomly split for total RNA isolation by KingFisher™ Duo Prime Purification System and the PicoPure Column. Four replicate extractions were made for each of the 6 mm and 2 mm punch size. qPCR was then performed.

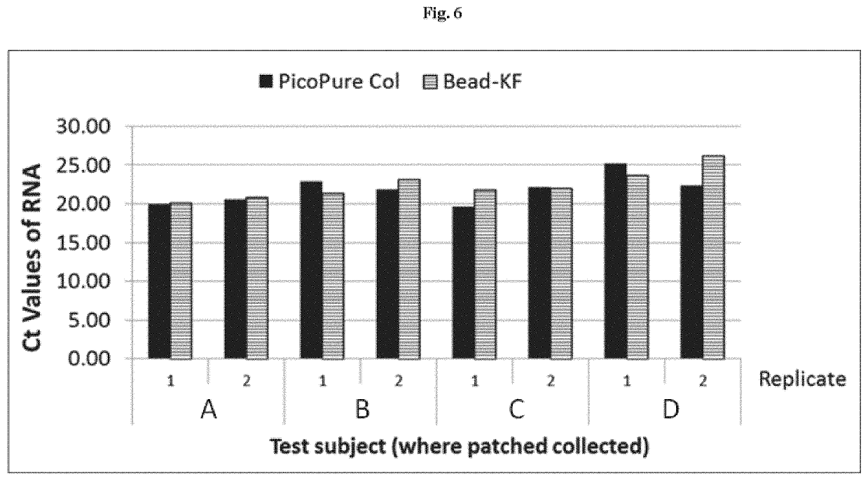

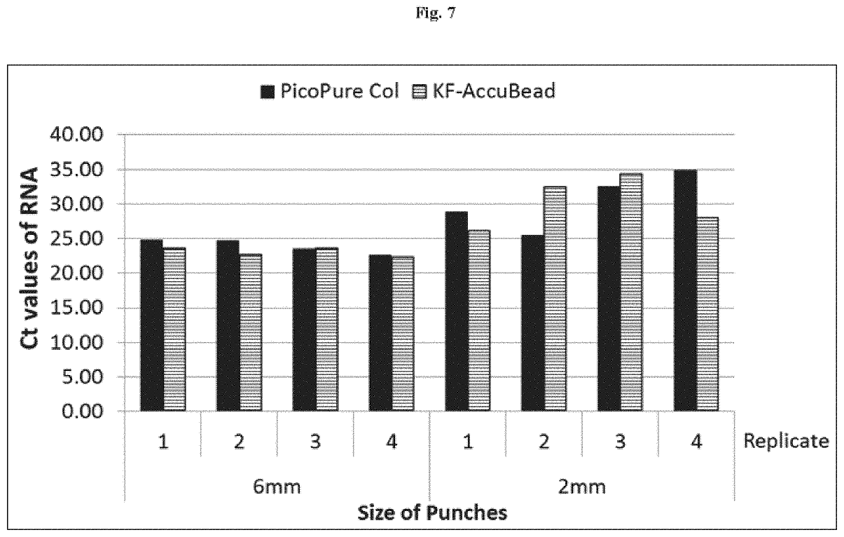

Referring to FIG. 7, threshold cycle (Cₜ) values of RNA (y-axis) was compared between RNA extracted using KingFisher™ Duo Prime Purification System (“KF-AccuBead,” horizontal hashed bars) and RNA extracted using the PicoPure Column (“PicoPure Col,” black bars). Cₜ values were compared from RNA extracted from 6 mm punch size and the 2 mm punch size. Comparison of C₁ values showed similar total RNA yield using the KingFisher™ Duo Prime Purification System and the PicoPure Column for the 6 mm punch size.

This example shows RNA extraction from punches of adhesive patches comprising skin samples using silica-coated magnetic beads.

### Example 9. RNA Yield and RNA Yield Distribution

RNA yield and RNA yield distribution was compared for RNA extracted using silica-coated magnetic beads and RNA extracted using column extraction.

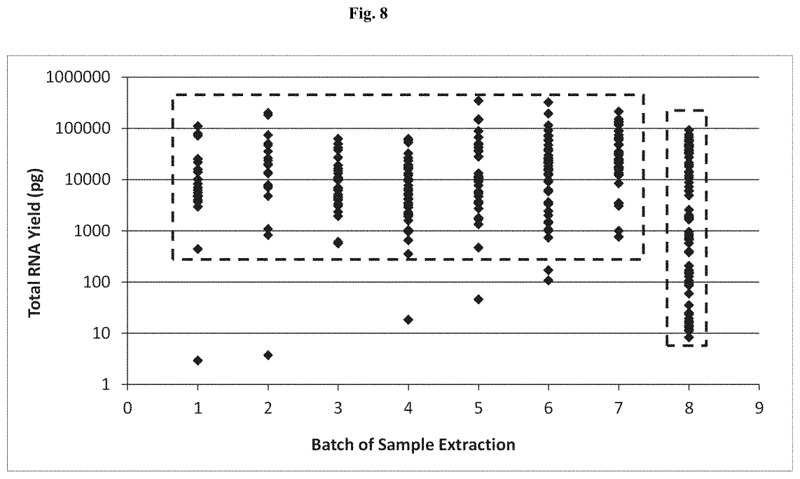

RNA was isolated and purified from skin samples according to previous examples. Referring to FIG. 8, RNA yield extracted using silica-coated magnetic beads (Samples 1-7 on x-axis) was compared to RNA extracted using the column extraction (Sample 8 on x-axis). There was an improved RNA yield (in picogram, y-axis) in samples extracted using the silica-coated magnetic beads (FIG. 8).

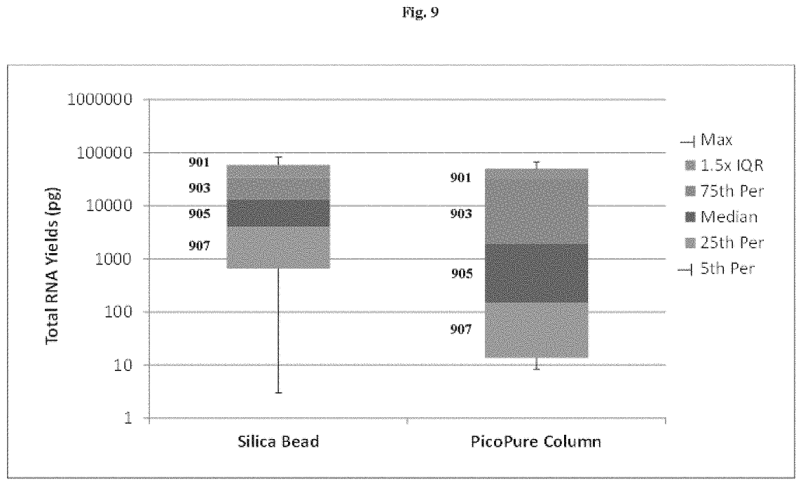

Referring to FIG. 9, the total RNA yield distribution in picogram (y-axis) was compared in RNA extracted using silica-coated magnetic beads (“Silica Bead”) and RNA extracted using column extraction (“PicoPure Column”). 901 shows the 1.5× interquartile range (IQR), 903 shows 75th Percentile (“75th Per”), 905 shows the median, and 907 shows 25th Percentile (“25th Per”) (FIG. 9). The IQR and median were compared between the two methods. There was improved total RNA yield using the silica-coated magnetic beads as compared to the column extraction.

This example shows RNA extraction using silica-coated magnetic beads result in improved RNA yield and RNA distribution.

### Example 10. Quality and Quantity of RNA Using Silica-Coated Magnetic Beads

Quality and quantity of RNA isolated using silica-coated magnetic beads was compared to RNA isolated using column extraction.

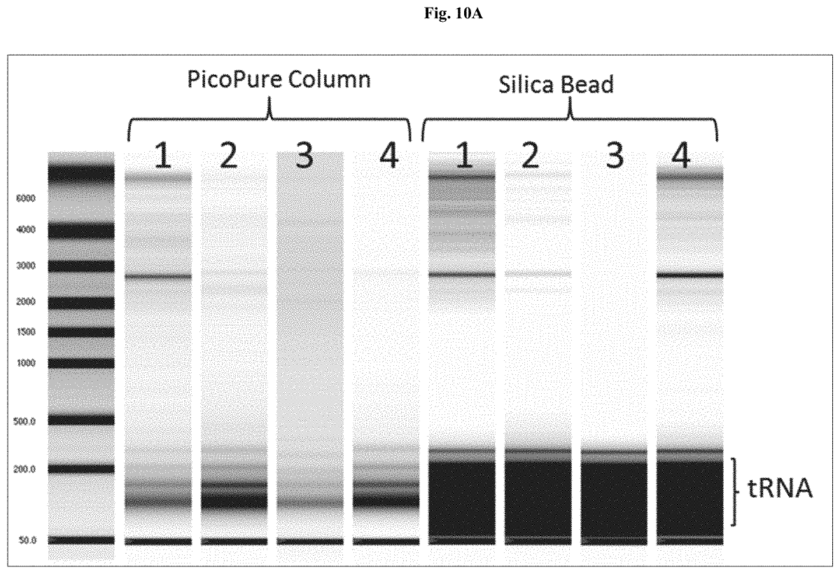

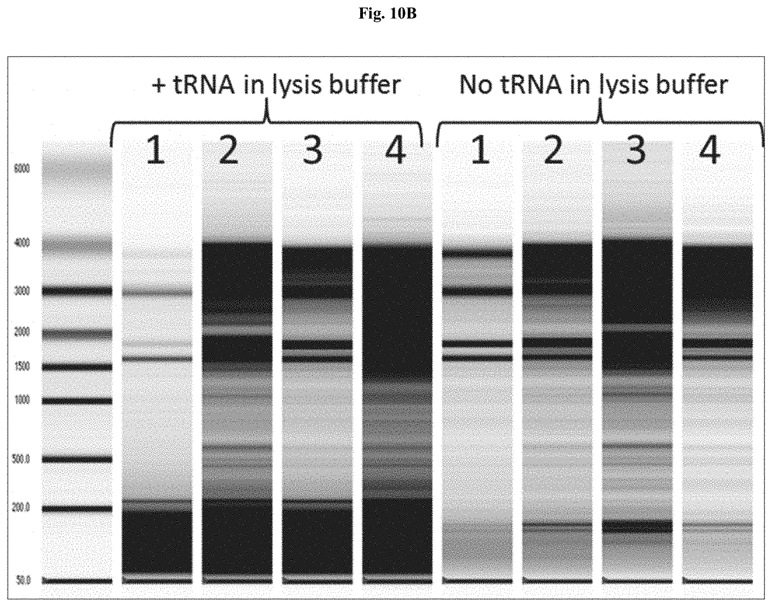

RNA was obtained from skin samples using adhesive patches according to previous examples. Referring to FIG. 10A, a first set of RNA samples 1-4 (on the left of the gel) were isolated using column extraction (“PicoPure Column”). A second set of RNA samples 1-4 (on the right of the gel) were isolated using silica-coated magnetic beads (“Silica Bead”). Both sets of RNA samples comprised transfer RNA (tRNA) in the lysis buffer. Both sets of RNA samples were run on an agarose gel. RNA isolated using silica-coated magnetic beads produced higher intensity of product bands (FIG. 10A). Samples 1-4 from the 2 methods (PicoPure Column and Silica Bead) are paired samples from 4 test subjects.

RNA was obtained from skin samples using adhesive patches according to previous examples. Referring to FIG. 10B, a first set of RNA samples 1-4 (on the left of the gel) comprised tRNA in the lysis buffer. The second set of RNA samples 1-4 (on the right of the gel) comprised no tRNA in the lysis buffer. Both sets of RNA samples were isolated using silica-coated magnetic beads. Without addition of tRNA to the lysis buffer, the method using the silica-coated magnetic beads produced a cleaner RNA product without tRNA in the elution (FIG. 10B).

This example shows RNA extraction using silica-coated magnetic beads result in improved quality of RNA.

### Example 11. Co-Isolation of Genomic DNA and RNA

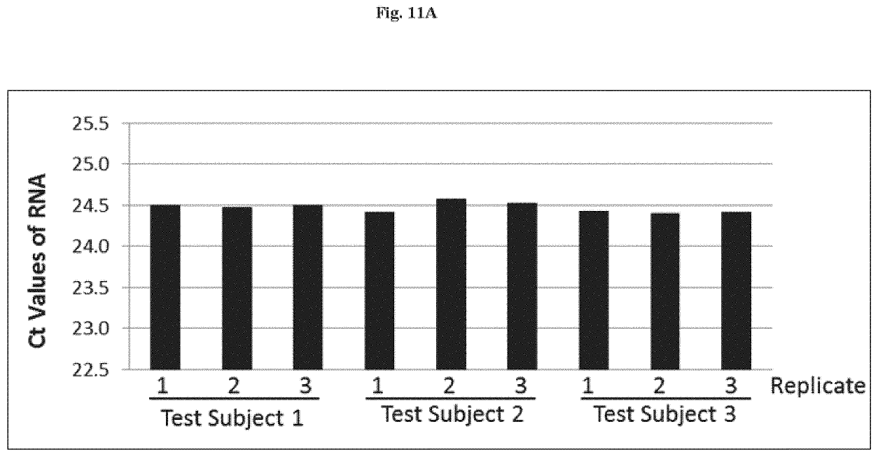

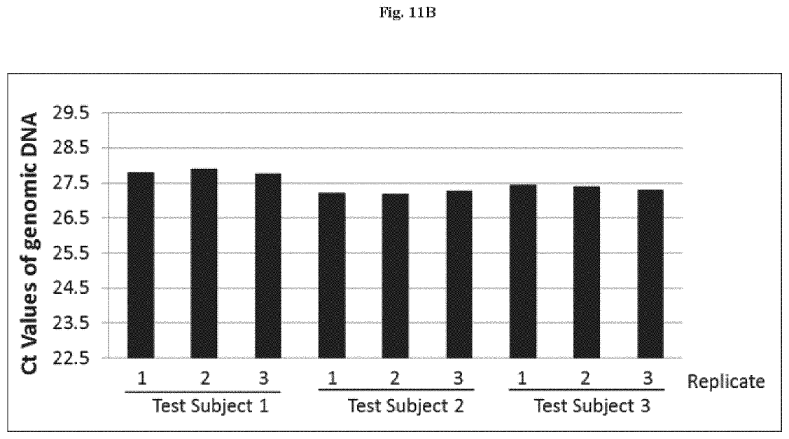

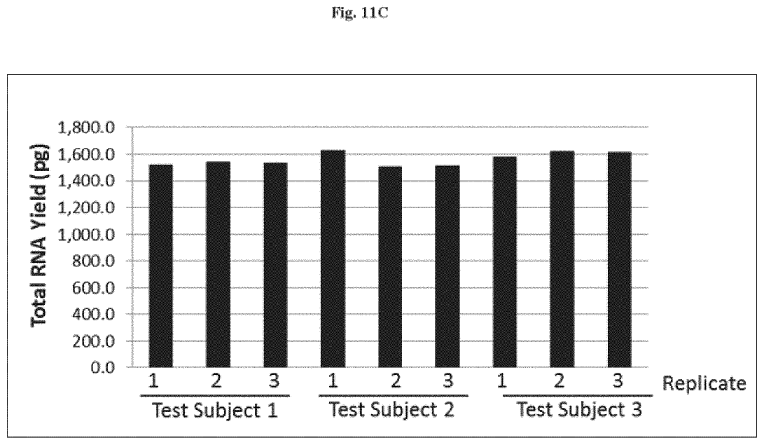

Samples were collected from forehead skin of 3 test subjects (“Test Subject 1,” “Test Subject 2,” “Test Subject 3”) using adhesive patches. Nucleic acids were isolated using silica-coated magnetic beads. The eluent products (2 uL) were assayed for total RNA (FIG. 11A) and for genomic DNA (gDNA) (FIG. 11B) by qPCR. Sample from Test Subject 1, Test Subject 2, and Test Subject 3 were analyzed in triplicate (FIGS. 11A-11B). Cₜ values (y-axis) were determined for the total RNA (FIG. 11A) and for the genomic DNA (FIG. 11B).

Referring to FIG. 11C and FIG. 11D, total yield was determined. Total RNA yield in picogram (y-axis) was determined for Test Subject 1, Test Subject 2, and Test Subject 3 (FIG. 11C). Total gDNA yield in picogram (y-axis) was determined for Test Subject 1, Test Subject 2, and Test Subject 3 (FIG. 11D). Sample from Test Subject 1, Test Subject 2, and Test Subject 3 were analyzed in triplicate (FIGS. 11C-11D).

This example shows that RNA and gDNA were co-isolated using silica-coated magnetic beads from the same sample.

### Example 12. Co-Isolation of Skin Microbiome DNA, Human RNA, and Human gDNA

Samples were collected from 4 test subjects using adhesive patches. The skin samples were collected from the forehead, inner arm, and the hand.

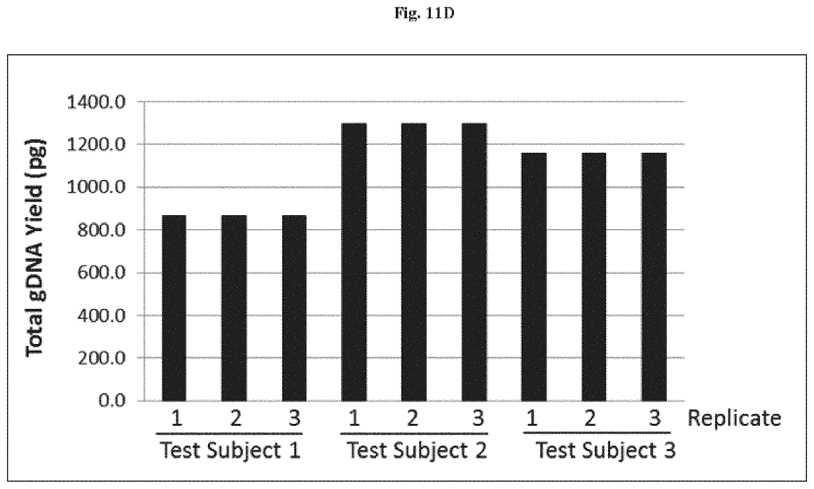

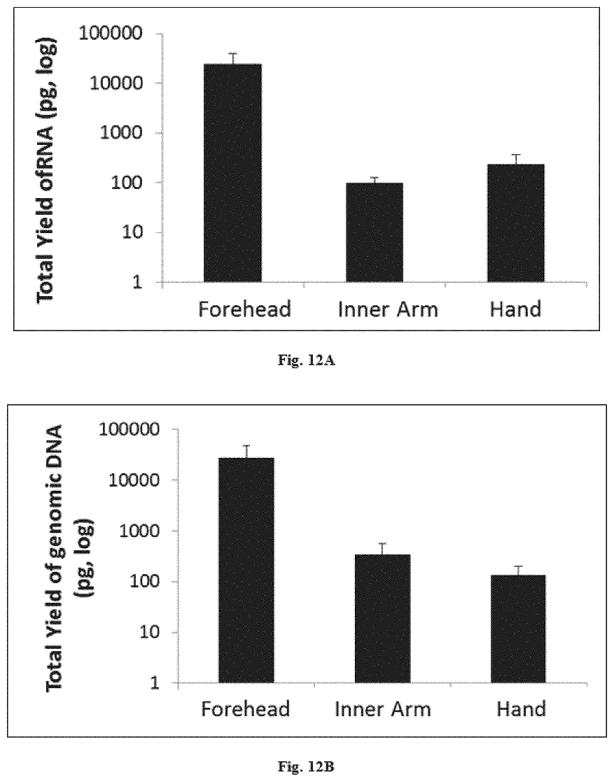

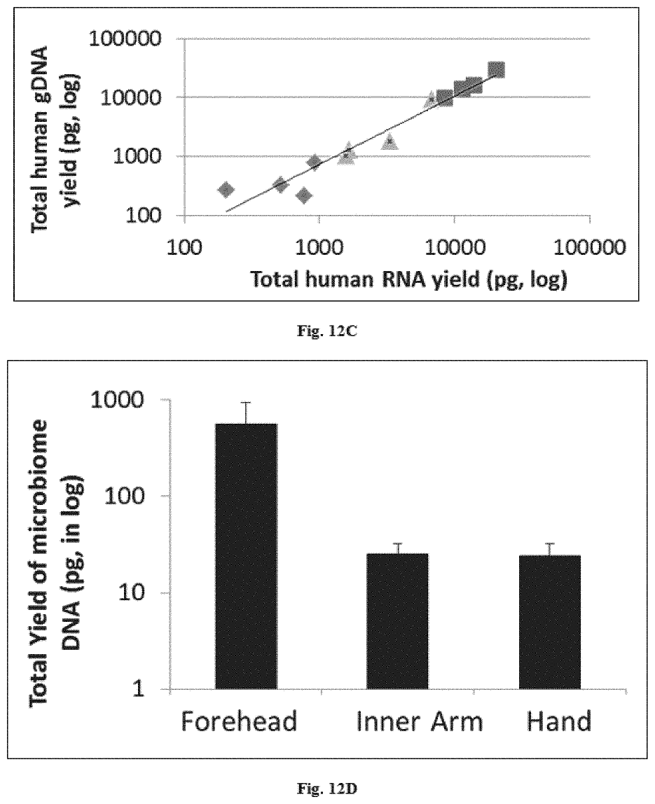

Referring to FIG. 12A, the total RNA yield in picogram (y-axis) was determined for samples collected from the forehead, inner arm, and the hand (x-axis). Total gDNA yield in picogram (y-axis) was determined for samples collected from the forehead, inner arm, and the hand (x-axis) (FIG. 12B). Yields for total RNA (FIG. 12A) and gDNA (FIG. 12B) are shown as mean±standard error (SE).

Referring to FIG. 12C, the linear correlation between human RNA yield (x-axis; pg, log) was compared to human gDNA yield (y-axis; pg, log).

Microbiome DNA was co-isolated from the skin samples collected using adhesive patches. Referring to FIG. 12D, the total yield (y-axis; pg, log) of microbiome DNA from skin samples collected from the forehead, inner arm, and the hand (x-axis) was determined.

This example shows that microbiome nucleic acids are co-extracted with human RNA and human genomic DNA from skin samples.

### Example 13. PCR Amplification of gDNA Co-Isolated Using Silica-Coated Magnetic Beads

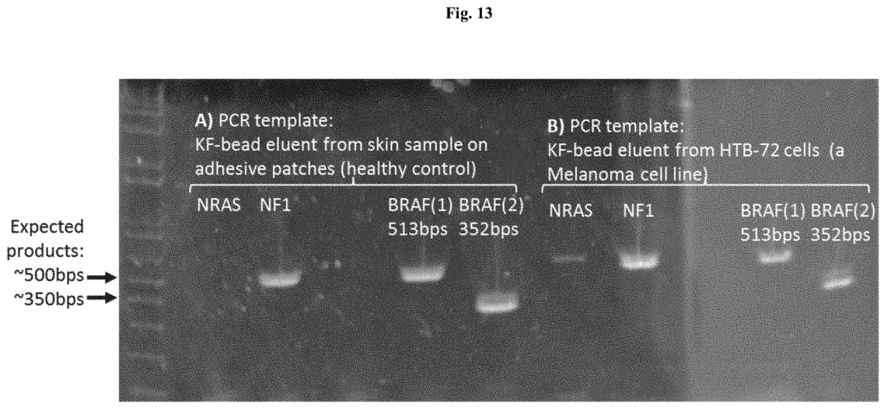

Genomic DNA (gDNA) was isolated from skin samples collected using adhesive patches. and from control cell line cells (HTB-72). The gDNA from the skin samples and from the HTB-72 cells were spiked to lysis buffer. Various genes were detected using PCR amplification including NRAS, NF1, and BRAF. Amplicon lengths ranged from 350 nucleotides to 530 nucleotides. Referring to FIG. 13, most products were amplified. NF1, 513 base pair BRAF, and 352 base pair BRAF were detected in skin samples isolated using silica-coated magnetic beads. NRAS, NF1, 513 base pair BRAF, and 352 base pair BRAF were detected in HTB-72 cells.

This example shows that gDNA is co-isolated using silica-coated magnetic beads and has improved quality, allowing for PCR amplification.

### Example 14. Molecular Diagnosis and Microbiome Analysis Using Adhesive Patch-Based Skin Biopsy

Subjects and Adhesive Patch Skin Biopsy Kit

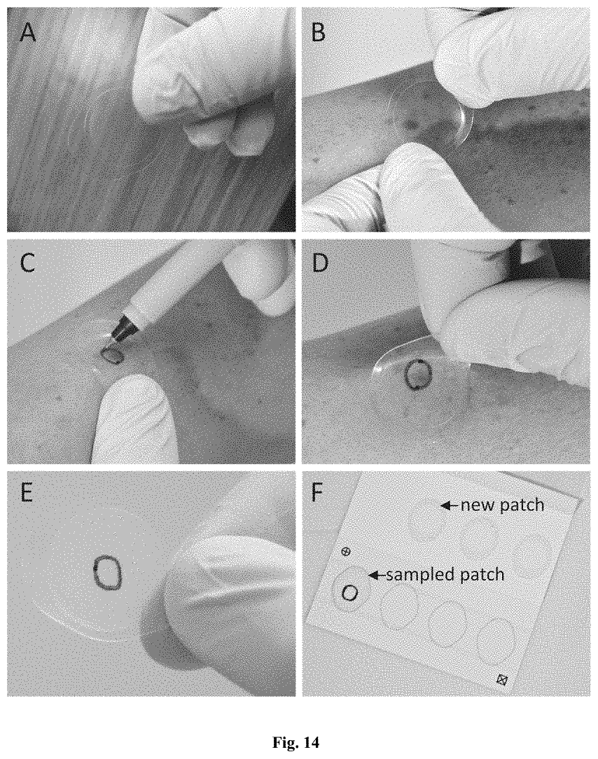

Subjects were adult males and females who met defined inclusion and exclusion criteria. Skin samples were collected using an Adhesive Patch Skin Biopsy (APSB) kit that contained a tri-fold sample collector (FIG. 14), a 70% alcohol preparation pad, a gauze pad, instructions for use (IFU), a laboratory requisition, and a courier envelope. The tri-fold collector comprised four transparent patches (round adhesive areas 19 mm in diameter) that were stored in a plastic bag.

Skin Sample Collection Procedure

A lesion or skin area of interest was cleaned with alcohol and hairs if present were removed using curved scissors. Each adhesive patch was placed on a cleaned and dried area of skin. A soft pressure using about 5 circular thumb motions was applied to fill the adhesive with epidermal skin cells. A lesion or area of interest was then demarcated on the patch. The patch was then removed and placed on the sample collector trifold. Four adhesive patches were used to harvest one skin sample. After the adhesive patch biopsy, the lower panel with harvested patches was folded and covered by the top panel to protect the harvested patches during storage and transportation.

Confirmation of Skin Tissue Collection

Successful collection of skin samples using adhesive patches was determined. Biomass of the harvested skin tissue on patches was measured. Using transmission electron microscopy (TEM), epidermal cells in the harvested skin tissue were visualized. Molecular analysis of total RNA or DNA isolated from the harvested skin tissue was also performed.

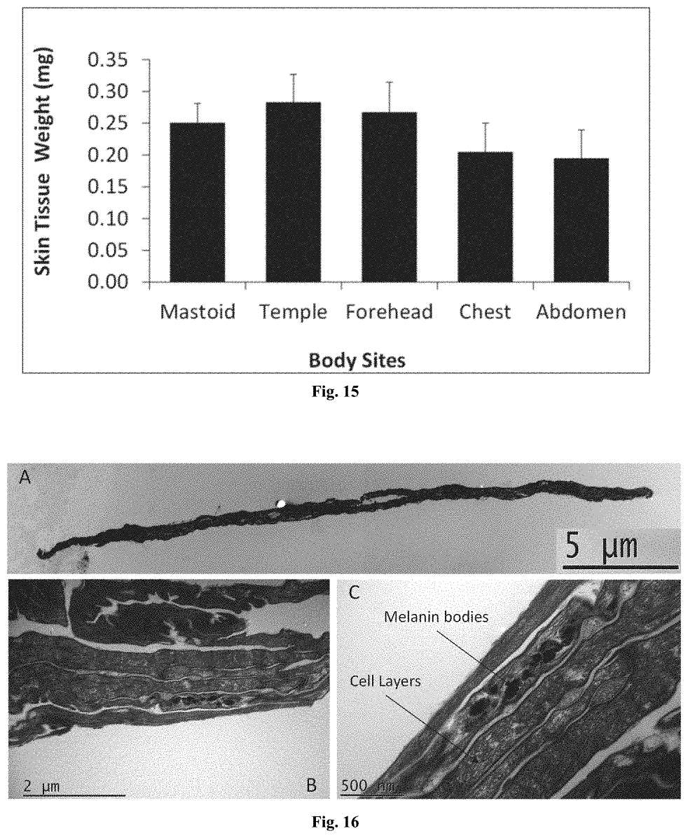

Biomass of harvested skin tissue on adhesive patches was determined through the weight changes (ΔW) of adhesive patches measured before (W0) and after (Ws) sample collection (ΔW=Ws−W0, per patch). Referring to FIG. 15, the biomass of non-invasively obtained skin tissue samples from 5 anatomical areas was determined. The 5 anatomical areas included the mastoid, temple, forehead, chest, and abdomen (x-axis). The sample biomass was measured as an increase in patch weight (ΔW), which is calculated as the weight of post-harvest patch (Ws) subtracted by the initial weight (W0) of the same patch before use (ΔW=Ws−W0). The mean skin tissue weight±standard error (SE) in milligram (y-axis) is shown in FIG. 15.

To prepare for TEM analysis of skin cells in harvested skin tissue on adhesive patches, the post-harvest adhesive patches were treated with methyl ethyl ketone (MEK) solution. The detached skin tissue was then collected on a Millipore filter connected to a syringe, washed and recovered in 3% buffered glutaraldehyde for processing via routine TEM. TEM images of the recovered skin tissue were taken at different magnifications. Referring to FIG. 16, TEM images show a representative section of skin tissue collected using adhesive patches. Low (4,400×, top panel), medium (20,000×, bottom left panel) and high (50,000×, bottom right panel) levels of magnification were used. At medium and high magnification, layers of intact skin cells (primarily keratinocytes) and intracellular structures such as melanin bodies were observed. These observations confirm the successful collection of epidermal skin tissue comparable to a very superficial shave biopsy procedure.

Tissues from individual patches were lysed in a modified lysis buffer from Norgen (Thorold, ON, Canada). Nucleic acids (RNA and DNA) were extracted using silica-coated magnetic beads on KingFisher Duo Prime (ThermoFisher Scientific, Waltham, Mass.). Total human RNA in the bead eluent was quantified by qPCR using human β-actin (ACTB) mRNA as a quantified marker. Total human genomic DNA (gDNA) in the same eluent was quantified using a standard gene copy number analysis qPCR Human ACTB gene was used as a quantified marker. Two microliters of bead eluent were used directly in qPCR. Quantities of total human gDNA in eluents were calculated from the Cₜ counts of ACTB from samples compared to the Cₜ counts of ACTB in standard curves prepared with human genomic DNA purchased from Promega (G3041; Promega, Madison, Wis.). In addition to human total RNA and gDNA, microbiome DNA in the bead eluent was also analyzed by qPCR. A pan-bacterial detection assay and 16S rRNA gene (Ba04230899_s 1, ThermoFisher Scientific) as a quantified marker were used. Quantities of microbiome DNA in the bead eluents were calculated from the Cₜ counts of 16S rRNA gene compared to the Cₜ counts of 16S rRNA gene in standard curves prepared with bacterial DNA (Ba04230899_s1, ThermoFisher Scientific). All qPCR reactions were performed using the 2× TaqMan Universal Master Mix from LifeTechnologies following the manufacturer's instruction. All reactions were carried out in triplicate on 384-well plates and run on an ABI 7900 PCR system (Life Technologies, Carlsbad, Calif.).

Stability of RNA in Tissue Stored on Patches after Harvesting

Stability of RNA in skin tissue embedded in the adhesive of patches after sample collection was determined. Stability of RNA was assessed by changes in copy numbers of amplifiable gene transcripts recovered from freshly harvested or stored samples from the same subject. Five subjects were used, and four temperature conditions were evaluated. See Table 5. Four samples were collected from the temple area. Two of the four samples were used for total RNA isolation (“Fresh”). Two of the four samples were stored (“Stored”) under a defined condition shown in Table 5 followed by RNA isolation. Total RNA was isolated and quantified following same procedures described above and using the same β-actin mRNA as a quantified marker.

TABLE 5
Experimental Design to Test the RNA Stability Under Different Storage Conditions

| | Number of | Total Number | Number of Patches for | Number of Patches for |
|---|---|---|---|---|
| Test Storage | Test | of Test | Initial Analysis | Final Analysis |
| Conditions | Subjects | Patches | (Day 0, Fresh) | (Day 7, Stored) |
| 25° C., 7 days | 5 | 5 × 4 | 5 × 2 (Day 0) | 5 × 2 (Day 7) |
| 40° C., 7 days | 5 | 5 × 4 | 5 × 2 (Day 0) | 5 × 2 (Day 7) |
| 60° C., 7 days | 5 | 5 × 4 | 5 × 2 (Day 0) | 5 × 2 (Day 7) |
| −80° C., 10 days | 5 | 5 × 4 | 5 × 2 (Day 0) | 5 × 2 (Day 10) |
| Total | 20 | 80 | 40 | 40 |

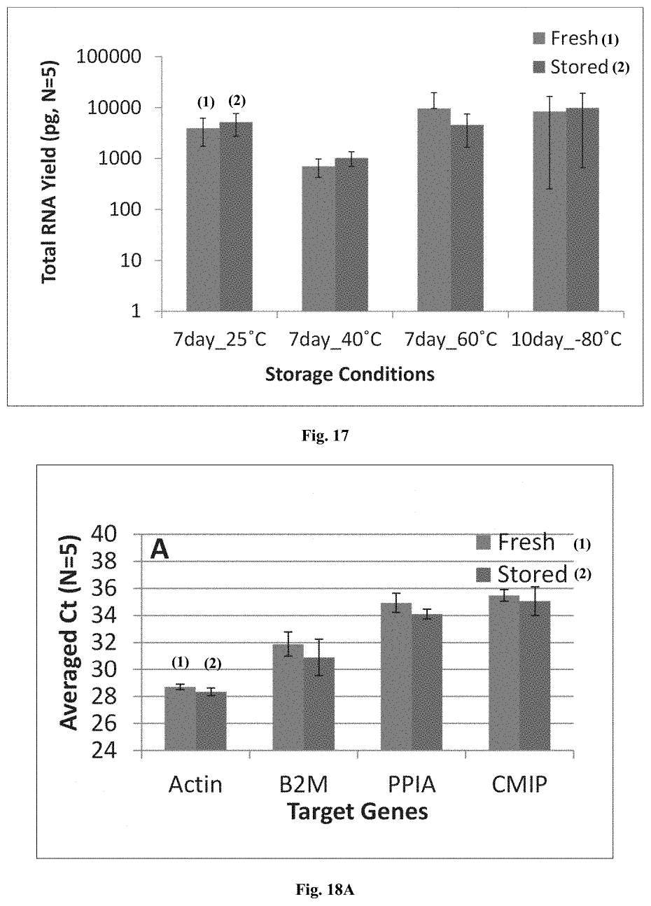

Referring to FIG. 17, total yield of RNA recovered from adhesive patches from the RNA stability study is shown. Values in gray bars (“Fresh,” gray bars (1), first bar on the left of the pair) represent averaged total RNA yields from freshly harvested tissues while values in dark gray bars (“Stored,” dark gray bars (2), second bar on the right of the pair) represent averaged total RNA yields from tissues stored on adhesive patches after harvesting. Four storage conditions were independently investigated. Though the total RNA yield varied among the different storage conditions, no statistically significantly difference (p<0.05) was seen between the fresh and stored samples in any of the storage conditions tested.

Quality of the isolated RNA from both fresh and stored skin tissues of different storage conditions was further evaluated using qPCR to detect 4 gene transcripts. The four genes included β-actin (ACTB), β-2-microglobulin (B2M), peptidylprolyl isomerase A (PPIA) and c-Maf inducing protein (CMIP). These genes represent genes with strong (ACTB), median (B2M), and weak (CMIP and PPIA) expression levels in human tissues. cDNA was prepared by reverse transcriptase with a normalized input of 40 picogram total RNA. The resulting cDNA was diluted and used in TaqMan qPCR gene expression assays. Gene expression assays of the 4 target genes were obtained from Life Technologies (ACTB Hs010606650_g1; B2M Hs00984230_m1; PPIA Hs04194521_s1; CMIP Hs00603125_m1). qPCR was performed following the manufacturer's instruction. All reactions were run in duplicate.

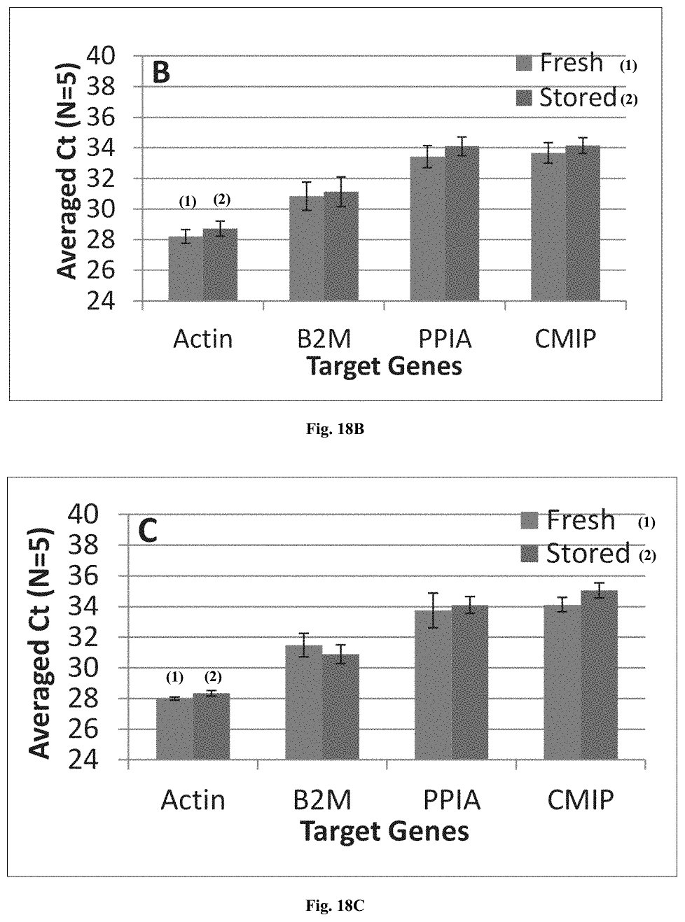

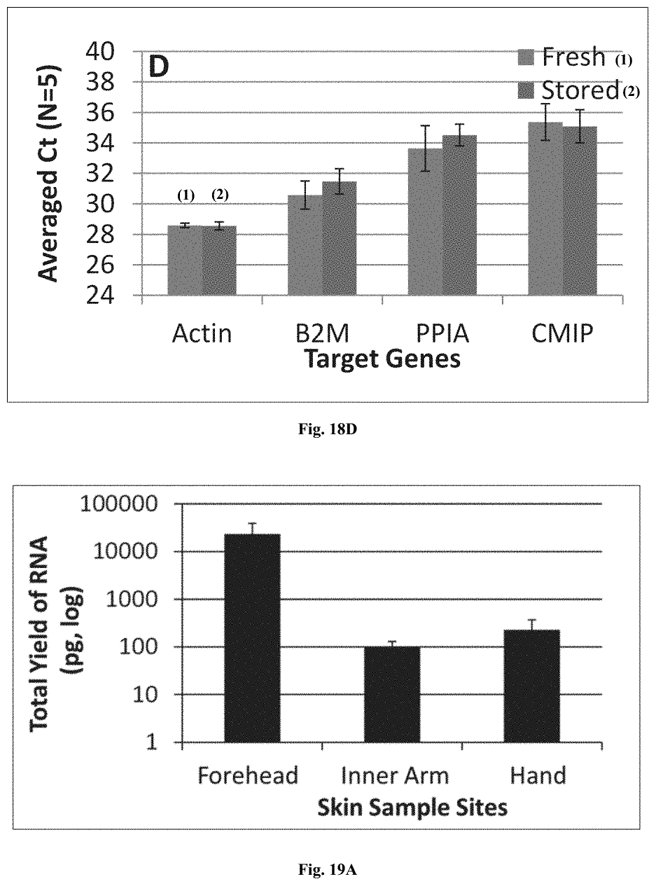

Referring to FIGS. 18A-18D, transcript analysis of the 4 genes in the isolated total RNA from fresh and stored skin tissue samples from the 4 storage conditions is shown. The 4 storage conditions include the following: 7 days at 25° C. (FIG. 18A), 7 days at 40° C. (FIG. 18B), 7 days at 60° C. (FIG. 18C), and 10 days at −80° C. (FIG. 18D). A similar copy number of the amplifiable transcripts in Fresh (gray bars (1), first bar on the left of the pair) versus Stored (dark gray bars (2), second bar on the right of the pair) samples. None of the Cₜ values from the 4 genes showed statistically significant difference (p>0.05) between Fresh and Stored samples in any of the 4 temperature conditions.

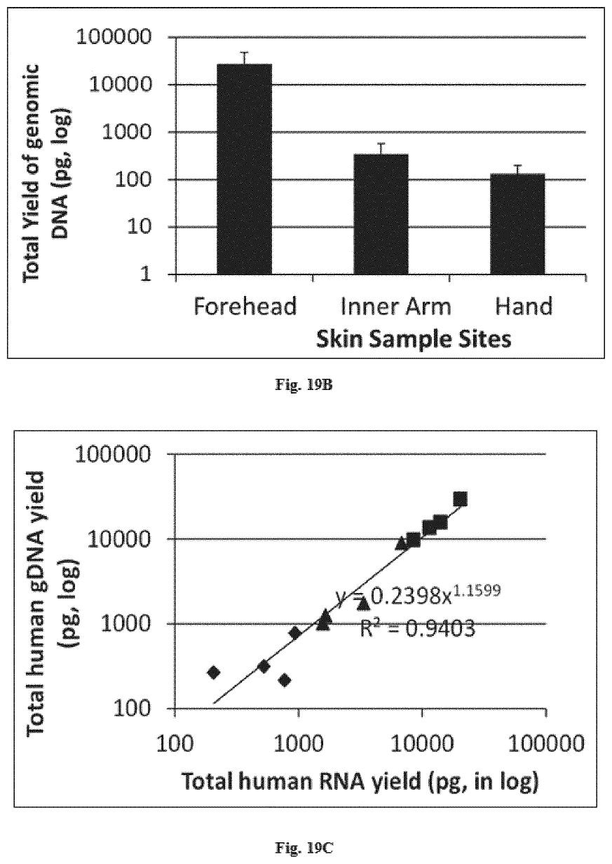

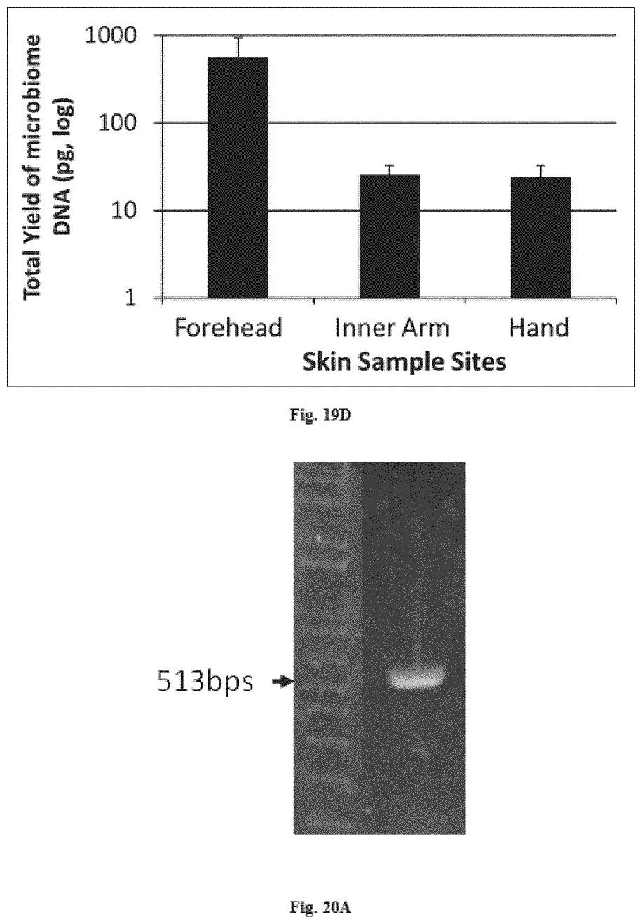

FIGS. 19A-19D show results of total nucleic acid extraction and quantification from skin samples collected from the forehead, the inner arm and the back of the hand from 4 test subjects. Both total human RNA (FIG. 19A) and human gDNA (FIG. 19B) was isolated from various anatomical locations using adhesive patches. The yield of total human RNA was 23.35±15.75 ng and human gDNA was 27.72±20.71 ng. The yield of human RNA and human gDNA was correlated linearly in each sample (FIG. 19C). Microbiome DNA was also detected in the same eluent from the skin tissue samples collected using adhesive patches (FIG. 19D). Total microbiome DNA yield was 576.2±376.8 pg.

The data indicates that collection methods described herein are also used for simultaneously obtaining skin microbiome samples.

Sanger Sequencing for Mutation Detection on Human gDNA

Sanger sequencing was used to detect human BRAF V600E gene mutation. PCR amplification of a 513 base pair length product covering human BRAF V600E mutation site was performed in a 25 uL PCR reaction containing 100 pigogram human gDNA from the above bead eluent. 200 nM of the forward primer (SEQ ID NO: 12) (TCTGGGCCTACATTGCTAAAATCTAA) and 200 nM of the reverse primer (SEQ ID NO: 13) (GTTGAGACCTTCAATGACTTTCTAGT) were used. Invitrogen™ Platinum™ TaqGreen Hot Start DNA polymerase (ThermoFisher Scientific) was added according to the manufacturer's instruction.

Following PCR, PCR products were first ExoSAP (Cat#78200, GE) treated and then used as templates for Sanger sequencing. Sequencing chromatogram files were examined using Chromas (version 2.01, University of Sussex, Brighton, United Kingdom).

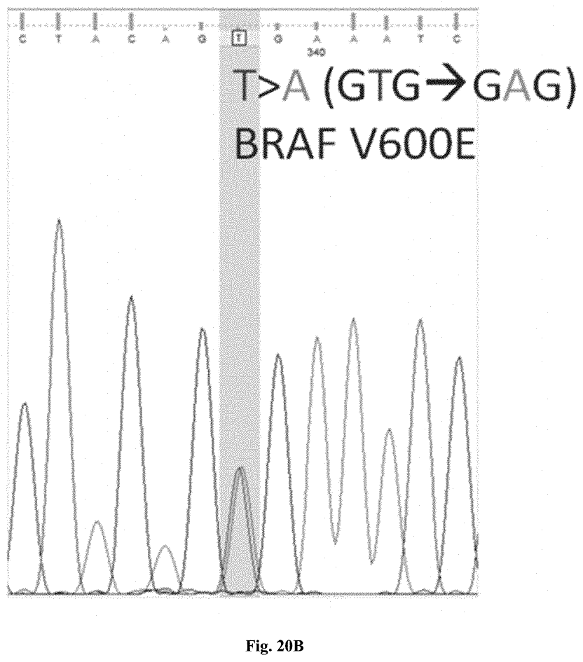

Referring to FIG. 20A, the isolated gDNA was used to successfully amplify longer PCR products such as the 513 base pair human BRAF gene exon. Sanger sequencing on this 513 base pair PCR product reliably detects BRAF V600E mutations (FIG. 20B) within adhesive patch skin samples.

These results demonstrate that quality, human gDNA for use in various genetic analysis are obtained using methods described herein.

Statistical Analysis

Statistical analyses were performed using Excel or R Tests for which the null hypothesis was no difference among procedures or conditions. Analyses were also performed with Student's t-test or analysis of variance. p-values less than 0.05 were considered significant.

This example shows extraction and co-isolation of microbiome nucleic acids and human nucleic acids using silica-coated magnetic beads from skin samples collected using adhesive patches. Following nucleic acid extraction, nucleic acids are used for determining expression level and mutational change of genes of interest.

### Example 15. Molecular Diagnosis using Expression and Mutational Change

Samples were processed similarly to Example 14 and analyzed for RNA expression and mutational change.

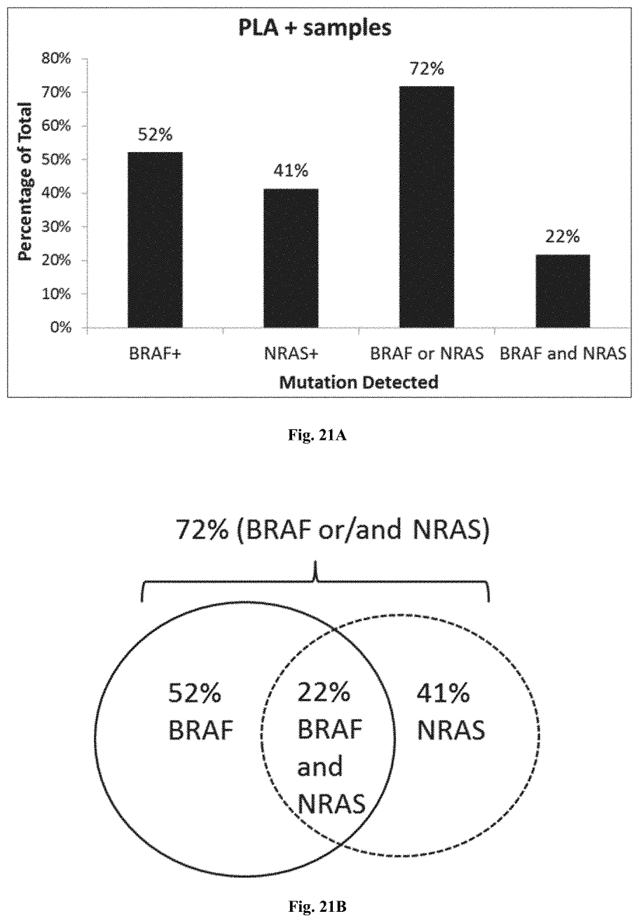

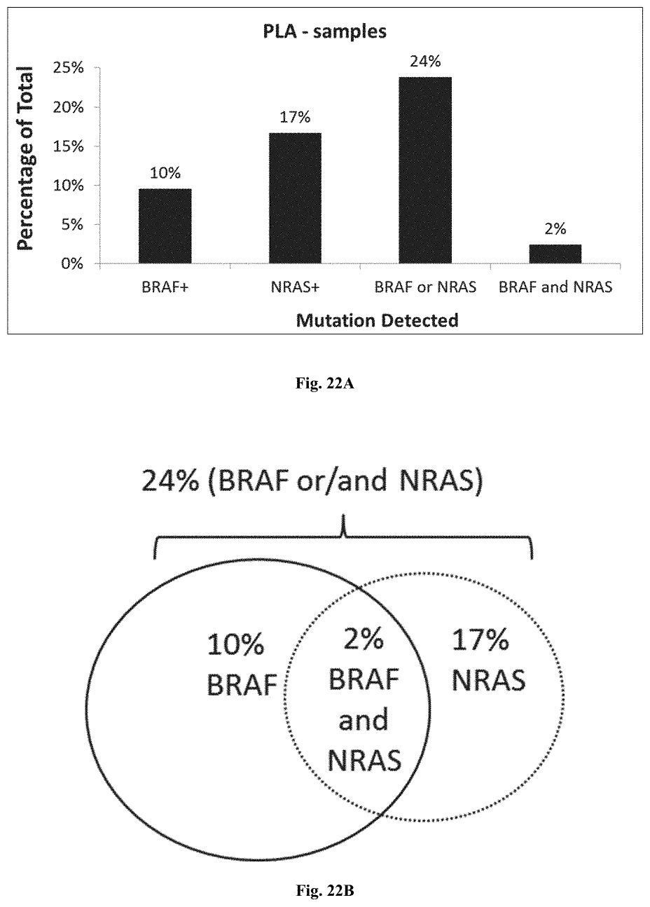

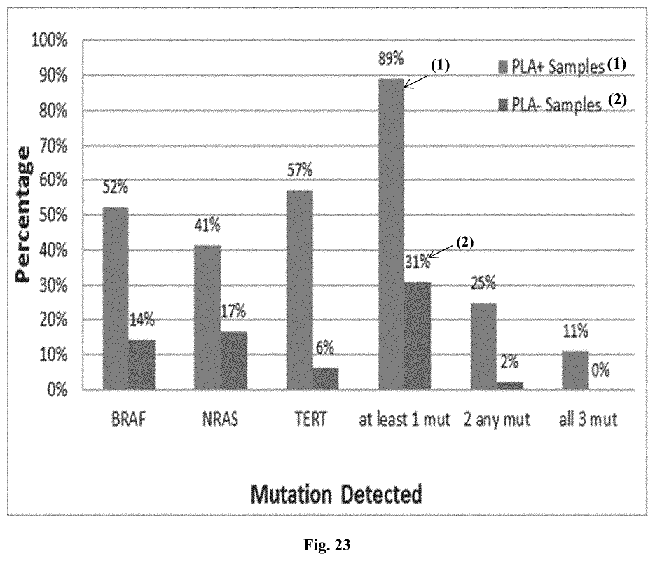

Samples were classified as PLA+ and PLA− according to PRAME or HNC expression (FIG. 21A, FIG. 22A, and FIG. 23). Mutational change in BRAF, NRAS, and TERT was determined by sequencing.

PLA+ samples were analyzed for mutations in BRAF, NRAS, BRAF or NRAS, and BRAF and NRAS (x-axis) as the percentage of the total (y-axis) (FIG. 21A). Referring to FIG. 21A, 52% of the PLA+ samples comprised BRAF mutations, 41% of the PLA+ samples comprised NRAS mutations, 72% of the PLA+ samples comprised BRAF or NRAS mutations, and 22% of the PLA+ samples comprised BRAF and NRAS mutations. Data from the graph are also represented in FIG. 21B.

PLA− samples were analyzed for mutations in BRAF, NRAS, BRAF or NRAS, and BRAF and NRAS (x-axis) as the percentage of the total (y-axis) (FIG. 22A). Referring to FIG. 22A, 10% of the PLA− samples comprised BRAF mutations, 17% of the PLA− samples comprised NRAS mutations, 24% of the PLA− samples comprised BRAF or NRAS mutations, and 2% of the PLA− samples comprised BRAF and NRAS mutations. Data from the graph are also represented in FIG. 22B.

Referring to FIG. 23, PLA+ and PLA− samples comprising BRAF mutations, NRAS mutations, TERT mutations, at least one mutation, any two mutations, or mutations in all three of BRAF, NRAS, and TERT was determined. In PLA+ samples (gray bars (1), first bar on the left of the pair), 52% comprised BRAF mutations, 41% comprised NRAS mutations, 57% comprised TERT mutations, 89% comprised at least one mutation, 25% comprised any two mutations, and 11% comprised mutations in BRAF, NRAS, and TERT. In PLA− samples (dark gray bars (2), second bar on the right of the pair), 14% comprised BRAF mutations, 17% comprised NRAS mutations, 6% comprised TERT mutations, 31% comprised at least one mutation, 2% comprised any two mutations, and 0% comprised mutations in BRAF, NRAS, and TERT.

### Example 16. Microbiome Detection of Adhesive Patch Skin Sample by PCR

Epidermal skin samples were collected with adhesive patches from 3 test subjects. 5 body sites (forehead, nose, cheek, arm (inner elbow) and lower leg) were collected from each subject. 4 adhesive patches from each body site were collected, with a total of 60 patches collected (3×5×4).

Methods from Example 14 were utilized to process the adhesive patch samples as well as the subsequent nucleic acid extraction and analysis. Total nucleic acids were extracted and processed from each patch separately utilizing the magnetic bead system described in Example 14 and were subsequently processed on a KingFisher Duo instrument. The nucleic acids were eluted in 50μL elution buffer for downstream qPCR analysis. The extraction contained human gDNA (genomic DNA) as well as gDNA microbiome (e.g., fungi and bacterial) present on the skin.

Real time qPCT was carried out for the 60 samples and the following targets were quantified:

Total human host skin cell—using human Beta actin (ACTB) gene (a housekeeping gene)

Number of total Fungi;

Number of total prokaryotic cells (bacteria)_16s rRNA TaqMan assay (purchased from LifeTechnologies);

Number of total prokaryotic cells (bacteria)_a separate 16s rRNA assay from a different sources with SYBR Green intercalating dye;

Number of Corynebacterium (a group of prokaryotic microbiome); and

Number of Staphylococcus (a group of prokaryotic microbiome).

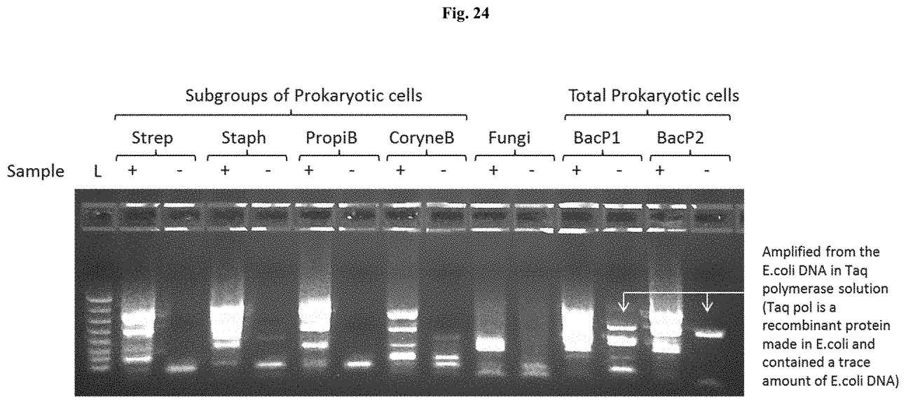

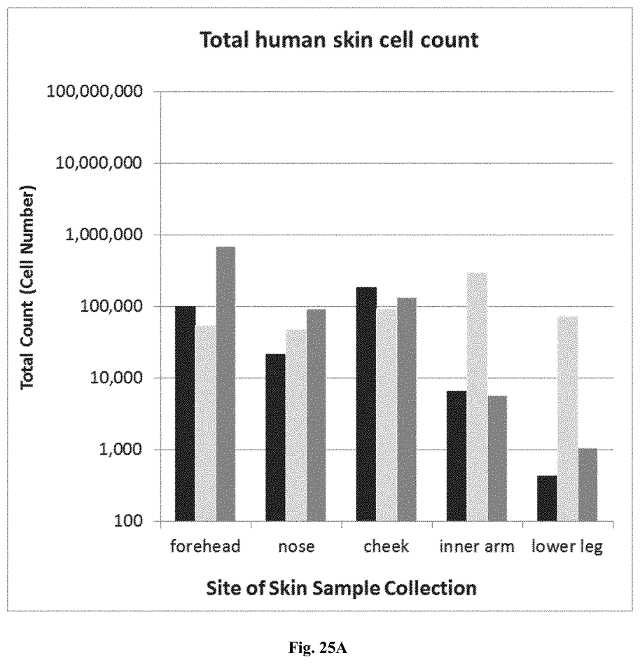

Both Fungi (eukaryotic cells) and prokaryotic microbiome (bacteria) were detected in the nucleic acid extraction from the epidermal skin tissue collected on adhesive patch. BacP1 and BacP2 are 2 separate assays to detect the total microbiome (both based on conserved regions of 16s rRNA of bacterial DNA) and both have detected the microbiomes from the sample. At least 4 types (genus) of bacteria were detected using target-specific PCR. Strep: Streptococcus; Staph: Staphylococcus; PropiB: Propionibacterium; CoryneB: Corynebacterium. ‘+’ with sample added to PCR; ‘−’: with water added to PCR (no template control). FIG. 24 illustrates the PCR detection of Streptococci (Strep), Staphylococci (Staph), Propionibacteria (PropiB), Corynebacteria (CoryneB) and Fungi from an adhesive patch collected epidermal skin sample.

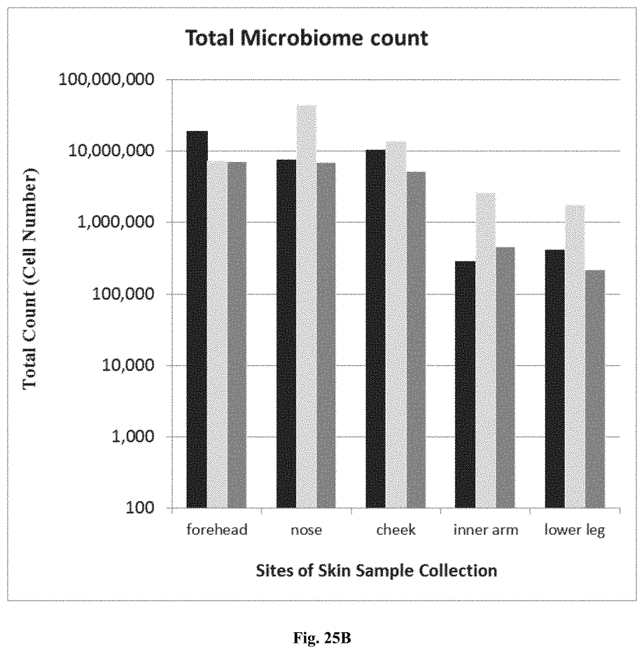

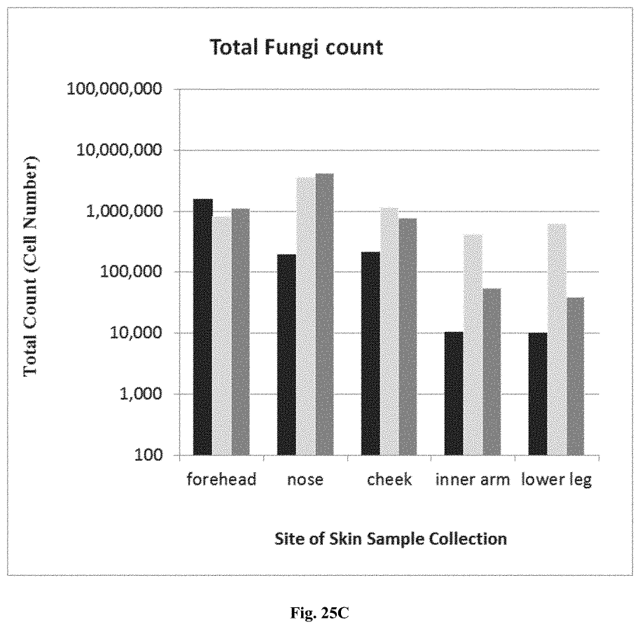

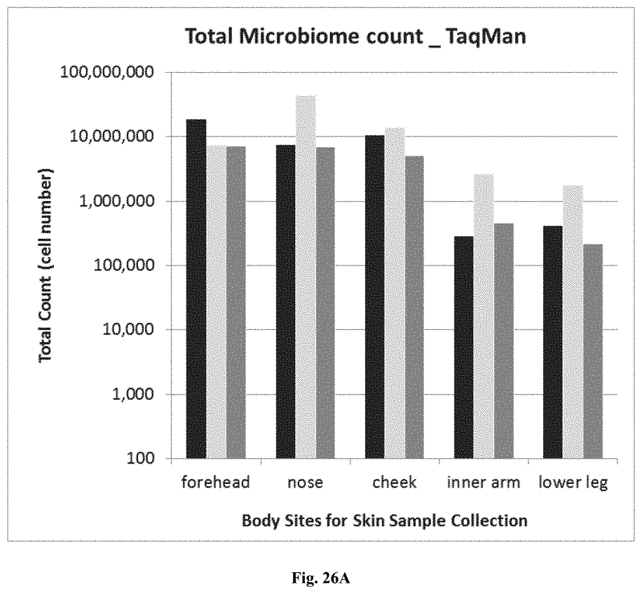

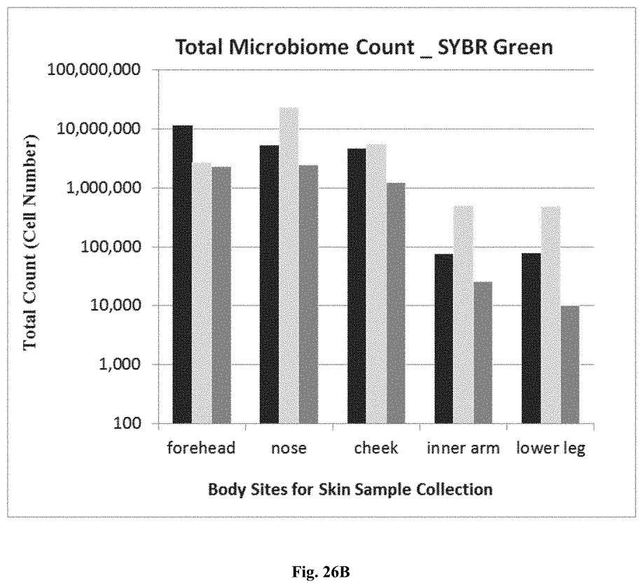

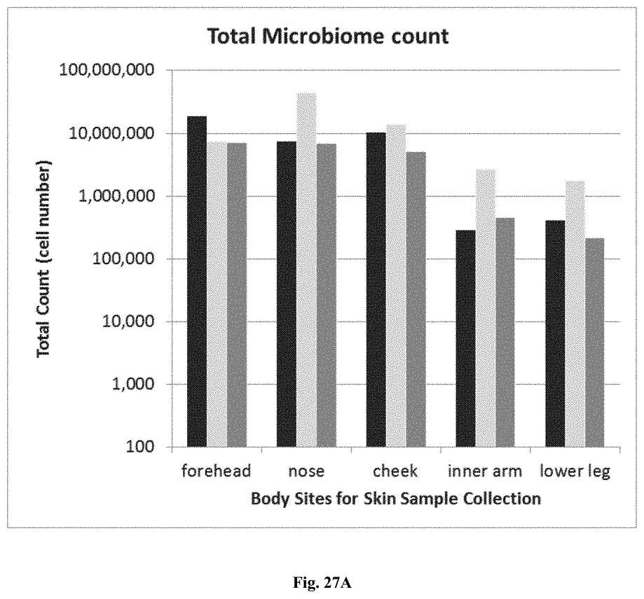

The total numbers of each target from each body site were calculated by combining the numbers from all 4 patches collected from the body site. The total numbers of host human skin cells, fungi and total microbiome varied between different body sites for sample collection (same on all 3 subjects) and the changes in host and microbiome numbers appear to correlate well in general. The total numbers of fungi and bacteria from each body sites studied were about 10 to 100 folds, respectively, more than that of the host skin cells regardless of the body site. FIG. 25A-25C illustrate the cell count obtained from each body site from human host skin (FIG. 25A), microbiome (FIG. 25B), and fungi (FIG. 25C).

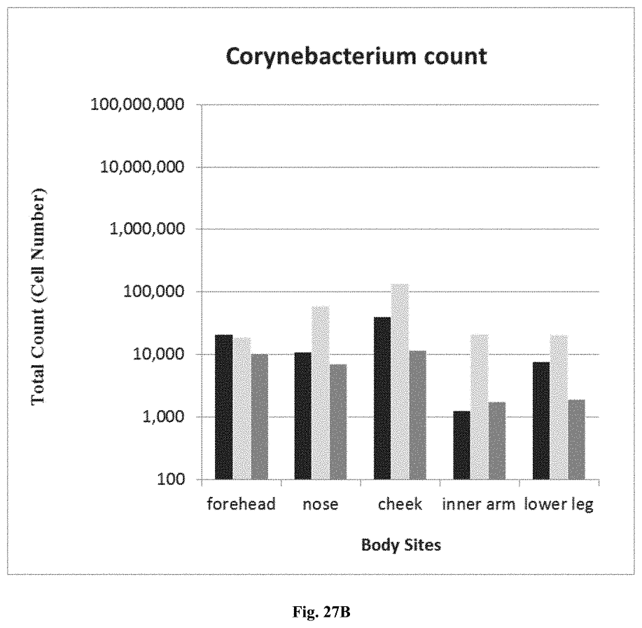

The total microbiome counts were determined with 2 separate real-time qPCR assays. Both qPCR assays were based on conserved regions in the 16s rRNA gene from prokaryotic microbiome DNA, but from different assay and primer designs. One of the qPCR assay used a TaqMan probe (a more specific probe) and the second qPCR used SYBR Green (intercalating dye to dsDNA PCR product) to detect the amplified microbiome PCR products. Both assays showed nearly the same microbiome counts in skin samples collected on the adhesive patches. FIG. 26A and FIG. 26B show the total microbiome counts determined using either the TaqMAN probe (FIG. 26A) or using the SYBR dye (FIG. 26B) for detection of the amplified product.

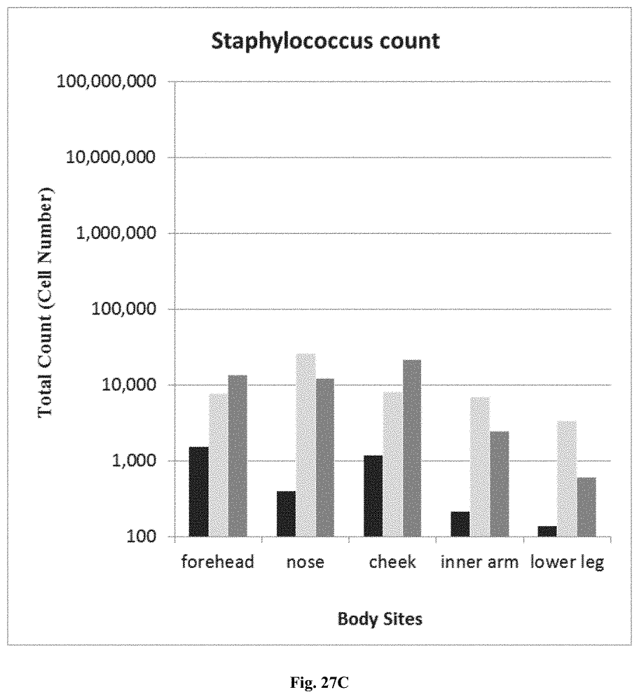

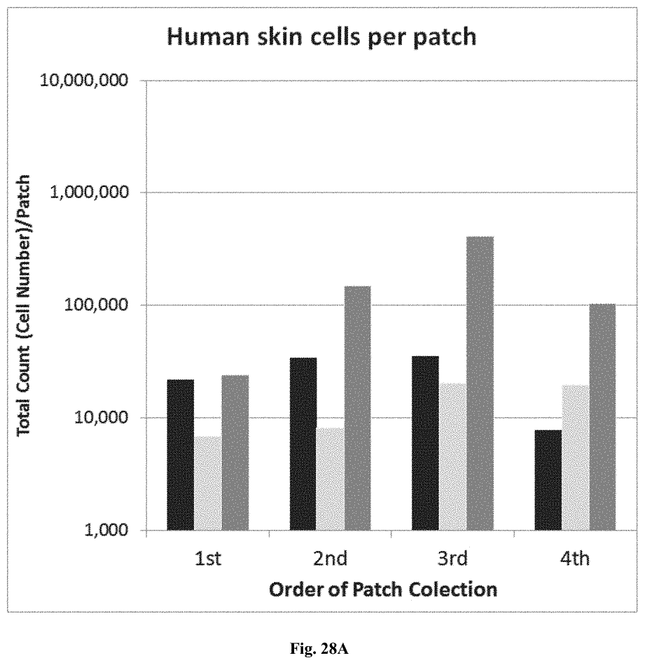

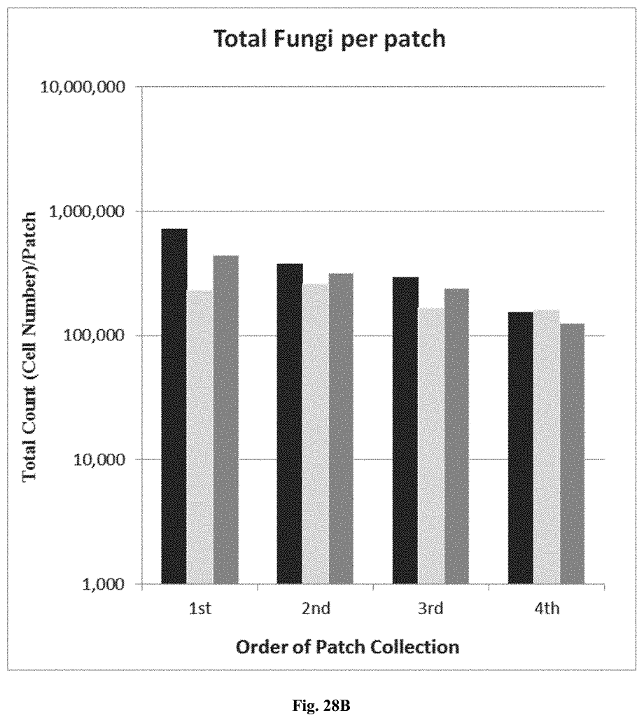

Corynebacterium and Staphylococcus were detected and quantifiable in the skin samples collected on the adhesive patches with target (genus)-specific qPCR assay on each target. FIG. 27A-FIG. 27C show the analysis of Corynebacterium, Staphylococcus, and the total microbiome numbers in skin samples harvested from different body sites from 3 test subjects. FIG. 27A shows the total microbiome count. FIG. 27B shows the total count from Corynebacterium. FIG. 27C shows the total count from Staphylococcus.

The numbers of fungi and microbiome on each individual patch showed a 10 to 100 fold difference relative to the number of host skin cell. The number of both fungi and microbiome decrease in the skin samples collected from deeper layers of skin (i.e., on each additional patch collection from the same test site) while the numbers of host human skin cells remained nearly unchanged, suggesting less microbiome residing in the deeper layers of skin. This trend of microbiome number changes was observed in the tested body sites with slight variations.

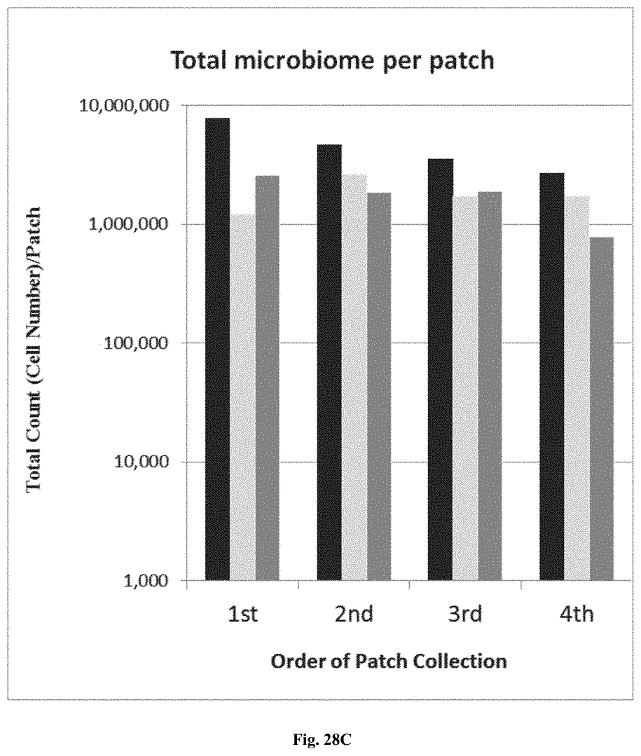

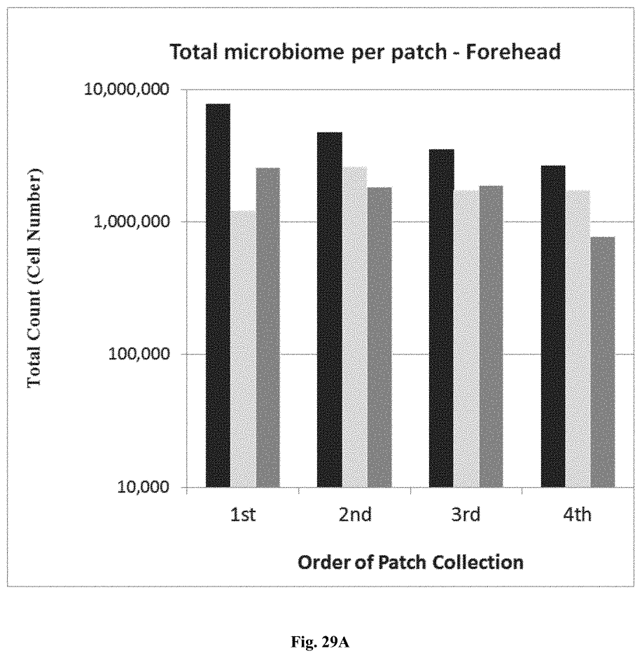

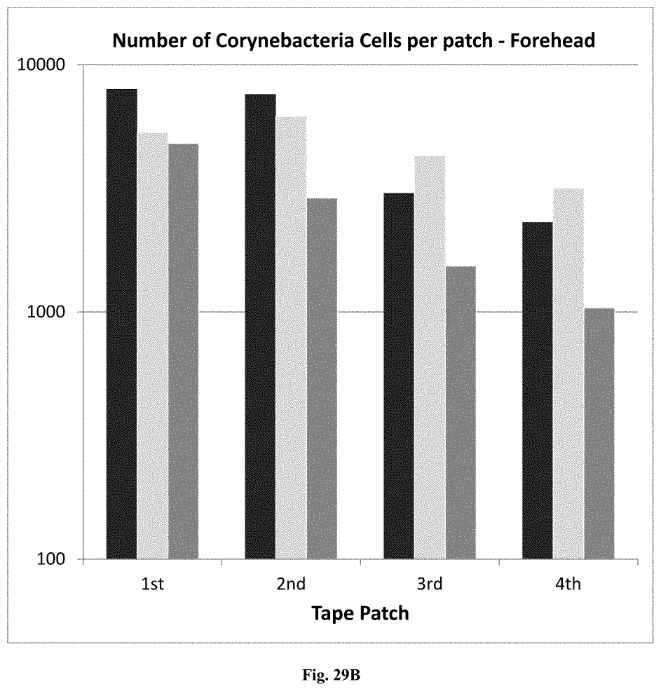

FIG. 28A-FIG. 28C show the analysis of the changes in the numbers of fungi and microbiome in samples collected from the different layers of skin, using forehead site as an example, from 3 test subjects (3 bar colors). FIG. 28A shows the analysis of the total human skin cells per patch. FIG. 28B shows the total fungi per patch. FIG. 28C shows the total microbiome per patch.

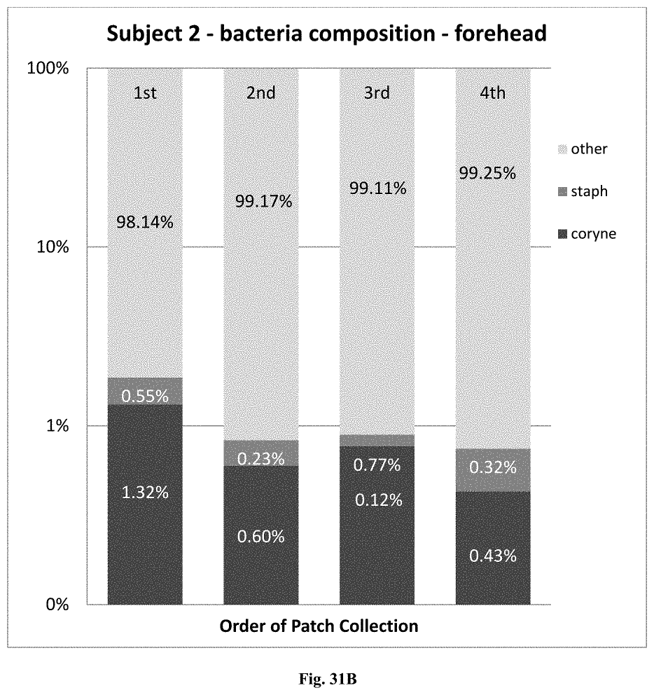

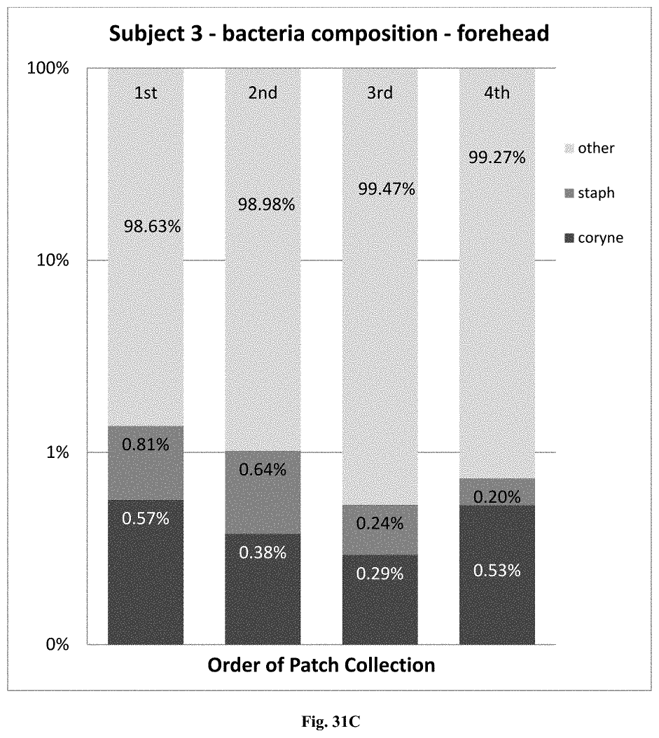

The changes of both Corynebacterium and Staphylococcus numbers in skin samples follow the same trend as that of the total microbiome count, and decrease in skin samples collected from deeper layers of skin. The detection of individual genus of microbiome further showed the kinetic changes of microbiome (composition, species and number) in different layers of epidermal skin (see FIG. 31A-FIG. 31C).

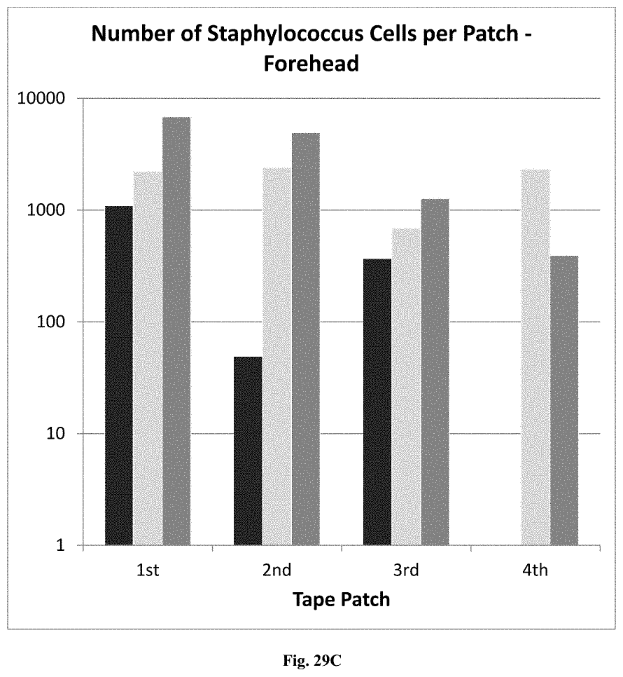

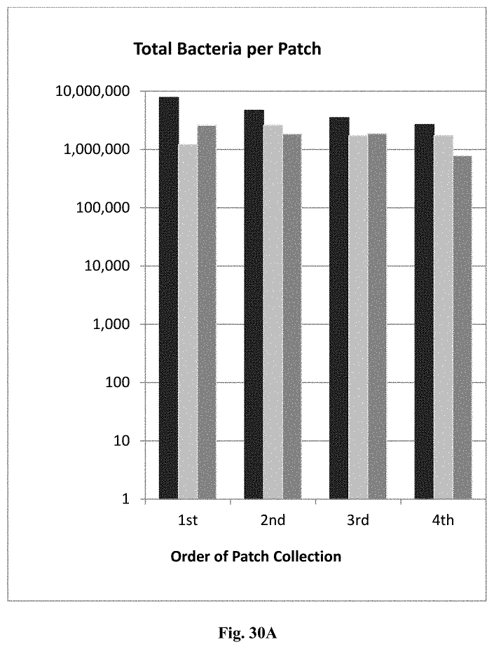

FIG. 29A-FIG. 29C show the analysis of the changes of Corynebacterium and Staphylococcus numbers in skin samples collected from different layers of skin, using forehead site as an example FIG. 29A shows the total microbiome per patch. FIG. 29B shows the number of Corynebacterium cells per patch. FIG. 29C shows the number of Staphylococcus cells per patch.

The numbers of Corynebacterium and Staphylococcus changed in skin samples collected from different epidermal layers. The total number of Corynebacterium and Staphylococcus contributes a small portion (<2%) of the total microbiome in the samples.

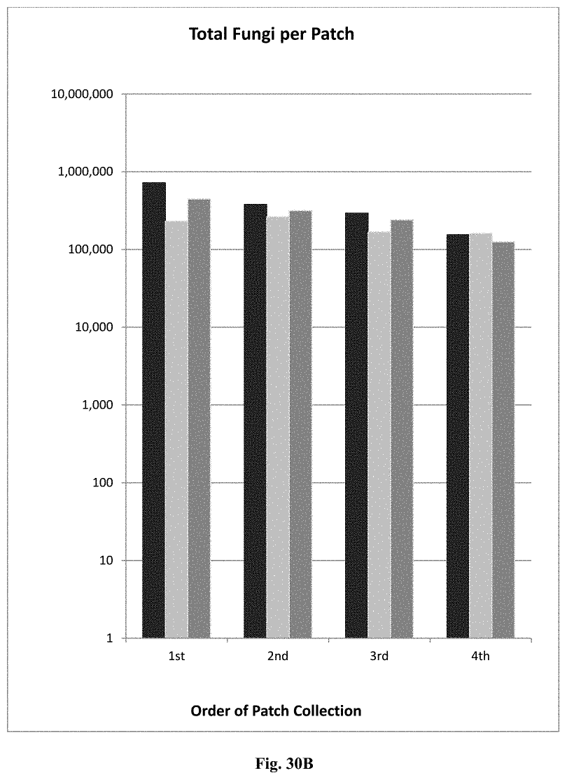

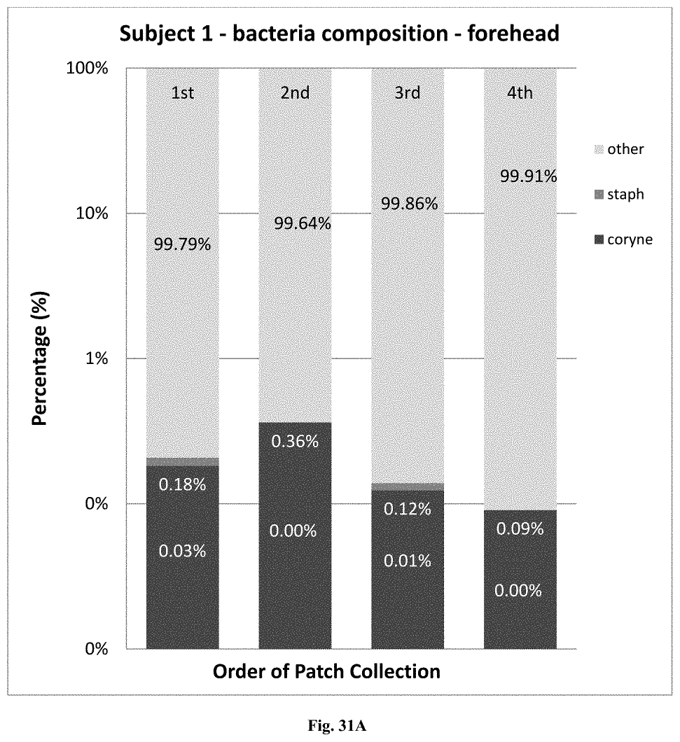

FIG. 30A and 30B illustrate total bacteria collected (FIG. 30A) or total fungi collected (FIG. 30B) at different skin depth level. The X-axis indicates the 1^{st}, 2^{nd}, 3_{rd} and 4^{th} sampling of the same skin area.

FIG. 31A-FIG. 31C show the analysis of the changes of Corynebacterium and Staphylococcus in percentage of total microbiome from the different layers of skin, using forehead site as an example, of the 3 test subjects. FIG. 31A illustrates the change in bacteria composition from the forehead region in Subject 1. FIG. 31B illustrates the change in bacteria composition from the forehead region in Subject 2. FIG. 31C illustrates the change in bacteria composition from the forehead region in Subject 3.

### Example 17. Methylation Detection of Target Genes Obtained from Adhesive Patch Skin Samples

Skin sample from adhesive patch was collected and processed as described above. The methylation status of keratin 10 gene KRT10 was determined using a methylation-specific PCR (MSP) method. The methylation status of keratin 14 gene KRT14 promoter region was determined using Sanger sequencing method. Table 6 illustrates the primer sequences utilized for this study.

TABLE 6

| | | | SEQ |
|---|---|---|---|
| | | | ID |
| Gene Name | Primer Name | Sequence | NO: |
| KRT10 | k10M For | AGTTTTCGTTTTCGTAG | 4 |
| | | TCGTC | |
| | k10M Rev | CGAATATAACCTCACCC | 5 |
| | | CG | |
| KRT10 | k10U For | GGAGTTTTTGTTTTTGT | 6 |
| | | AGTTGTT | |
| | k10U Rev | AACCAAATATAACCTCA | 7 |
| | | CCCCA | |
| KRT14 | k14 Prom For | GGTGTGGTGGATGTGAG | 8 |
| (promoter) | | ATTT | |
| | k14 Prom Rev | CTTTCATCACCCACAAA | 9 |
| | | CTAAC | |
| KRT14 | k14 For_Seq1 | ATAGGGAGGAGATTAGG | 10 |
| (promoter) | | GTTT | |
| | k14 For_Seq2 | GGGAGGTTTGTTTGTGT | 11 |
| | | TTAAGG | |

Methylation Study of KRT10: Skin samples from two patients were obtained and the samples were labelled as Sample ID 4166 and Sample ID 4247. Two sets of PCR sequencing were performed for each sample. The first set of PCR sequencing was performed on methylated gDNA comprising the KRT10 gene and the primers used were denoted by “M” (e.g., k10M For). The second set of PCR sequencing was performed on unmethylated gDNA comprising the KRT10 gene (i.e., after bisulfite treatment) and the primers used were denoted by “U” (e.g., k10U For). qPCR was performed to generate Ct values from each set of experiments. The percentage of DNA methylation was then calculated from the Ct values using the following equations:

% methylation=(1/(1+2^{(-ΔCt)}))* 100

ΔCt=Ct.U−Ct.M

TABLE 7
illustrates the Ct values, ΔCt values, and the
percentage of methylation of the two samples.

| Sample ID | Ct | |
|---|---|---|
| 4166 | 34.15 | (Ct.M) |
| 4166 | 29.36 | (Ct.U) |
| 4247 | 32.93 | (Ct.M) |
| 4247 | 29.22 | (Ct.U) |

ΔCt (CtU-CtM)

| | |
|---|---|
| 4166 | −4.80 |
| 4247 | −3.71 |

% Methylation

| | |
|---|---|
| 4166 | 3.5% |
| 4247 | 7.1% |

Methylation Study of KRT14: Sanger sequencing was utilized to detect the methylation status or percentage of KRT14. The percentage of methylation was calculated based on the number of mCG and TG.

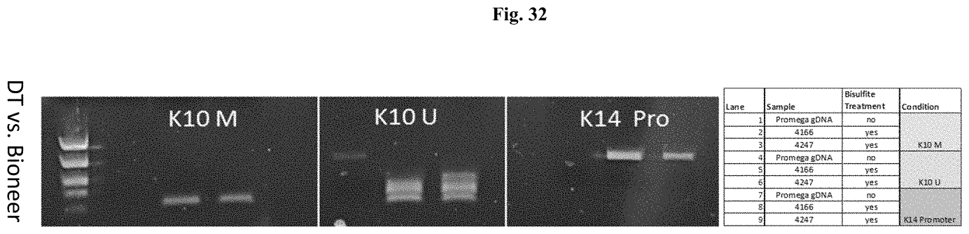

FIG. 32 depicts a gel electrophoresis of polymerase chain reaction (PCR) products of KRT10 and KRT14.

### Example 18. Co-Isolation of RNA and DNA Using Silica-Coated Magnetic Beads

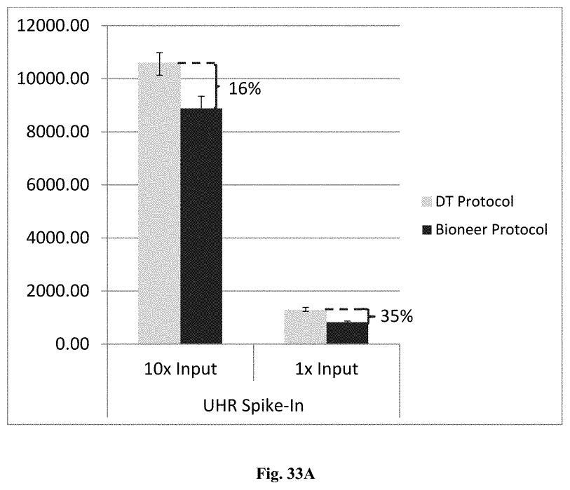

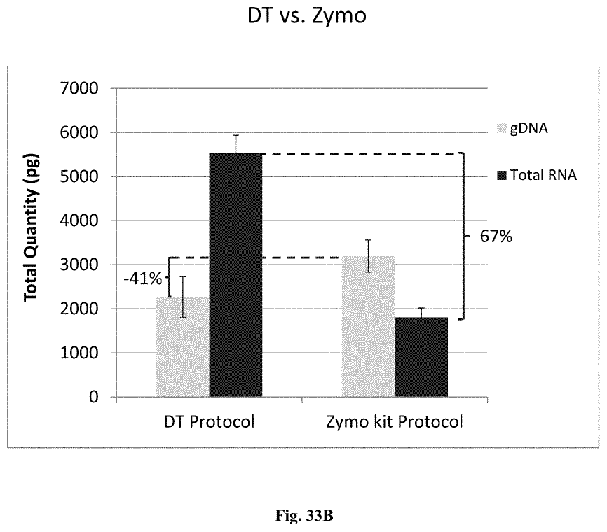

The percentage of RNA and DNA recovery utilizing the method described herein was compared with two commercial methods for RNA and DNA recovery. FIG. 33A shows results from a RNA recovery test in which universal human RNA (UHR) were spiked to the lysis buffers (in 2 input levels) and extracted with the method described herein vs. an extraction method described by Bioneer. As shown in the figure, about 35% (at a 1× input level) and 16% (at a 10× input level) more RNA were isolated using the method described herein than with the Bioneer method.

FIG. 33B shows results from DNA and RNA extraction from skin samples collected on adhesive patch using the method described herein in comparison with an extraction method described by Zymol Research (Cat. D4100-2-3). As shown here, about 67% more of total RNA was isolated using the method described herein.

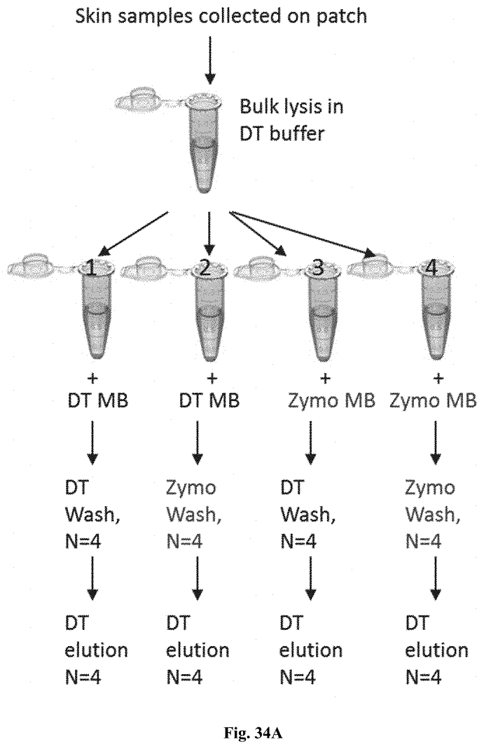

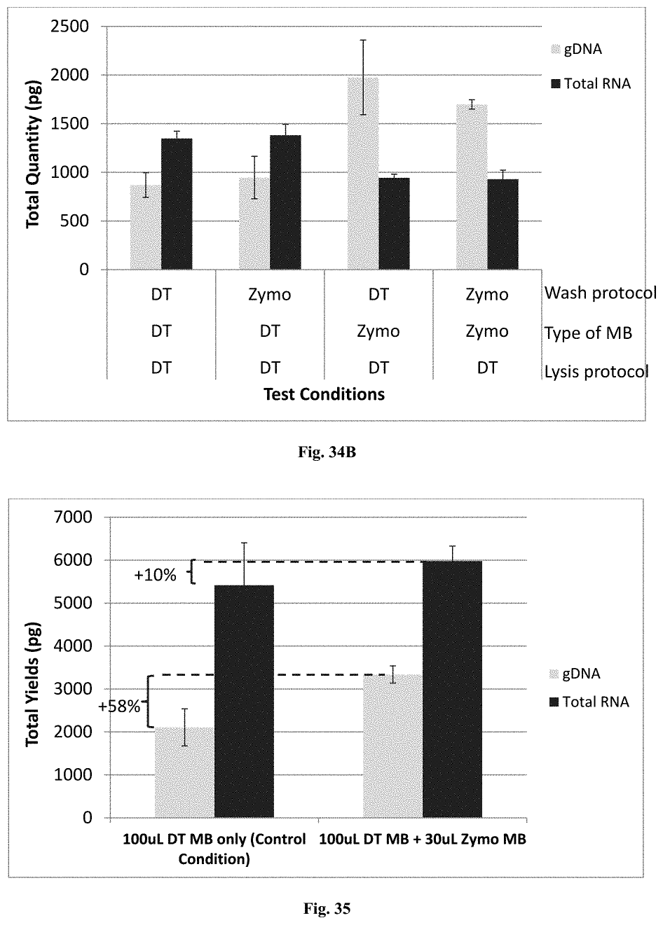

FIG. 34A illustrates an exemplary test design and procedure, where a bulk lysate of skin sample in lysis buffer was aliquoted to 4 groups of tubes, receiving either the magnetic beads described in Example 5 (referenced as DT MB in the FIG. 1, 2) or the magnetic beads from Zymo Research (referenced as Zymo MB in the FIG. 3, 4). After incubation, the magnetic beads in these tubes were washed either in a wash buffer prepared in-house or in a wash buffer from Zymo Research, and finally all samples were eluted in an in-house elution buffer. Total RNA and gDNA from all eluents were shown in FIG. 34B.

Based on the results from FIG. 34B, different volume ratios of the DT MB and Zymo MB were tested. FIG. 35 illustrates gDNA and total RNA extraction utilizing a 100 μL DT MB:30 μL Zymo MB ratio compared to the control, which contains 100 μL of DT MB.

### Example 19. Amino Acid Sequences

Table 8 shows exemplary sequences of the genes of interest disclosed herein.

TABLE 8
Amino Acid Sequences.

| SEQ | | | |
|---|---|---|---|
| ID | | Accession | |
| NO | Protein | No. | Amino Acid Sequence |
| 1 | BRAF | NP_001341538.1 | MAALSGGGGGGAEPGQALFNGDMEPEAGAGAGAAASSAADPAIPE |
| | | | EVWNIKQMIKLTQEHIEALLDKFGGEHNPPSIYLEAYEEYTSKLDAL |
| | | | QQREQQLLESLGNGTDFSVSSSASMDTVTSSSSSSLSVLPSSLSVFQN |
| | | | PTDVARSNPKSPQKPIVRVFLPNKQRTVVPARCGVTVRDSLKKALM |
| | | | MRGLIPECCAVYRIQDGEKKPIGWDTDISWLTGEELHVEVLENVPL |
| | | | TTHNFVRKTFFTLAFCDFCRKLLFQGFRCQTCGYKFHQRCSTEVPL |
| | | | MCVNYDQLDLLFVSKFFEHHPIPQEEASLAETALTSGSSPSAPASDSI |
| | | | GPQILTSPSPSKSIPIPQPFRPADEDHRNQFGQRDRSSSAPNVHINTIEP |
| | | | VNIDDLIRDQGFRGDGGSTTGLSATPPASLPGSLTNVKALQKSPGPQ |
| | | | RERKSSSSSEDRNRMKTLGRRDSSDDWEIPDGQITVGQRIGSGSFGT |
| | | | VYKGKWHGDVAVKMLNVTAPTPQQLQAFKNEVGVLRKTRHVNIL |
| | | | LFMGYSTKPQLAIVTQWCEGSSLYHHLHIIETKFEMIKLIDIARQTAQ |
| | | | GMDYLHAKSIIHRDLKSNNIFLHEDLTVKIGDFGLATVKSRWSGSH |
| | | | QFEQLSGSILWMAPEVIRMQDKNPYSFQSDVYAFGIVLYELMTGQL |
| | | | PYSNINNRDQIIFMVGRGYLSPDLSKVRSNCPKAMKRLMAECLKKK |
| | | | RDERPLFPQILASIELLARSLPKIHRSASEPSLNRAGFQTEDFSLYACA |
| | | | SPKTPIQAGGYGEFAAFK |
| 2 | NRAS | NP_002515.1 | MTEYKLVVVGAGGVGKSALTIQLIQNHFVDEYDPTIEDSYRKQVVI |
| | | | DGETCLLDILDTAGQEEYSAMRDQYMRTGEGFLCVFAINNSKSFAD |
| | | | INLYREQIKRVKDSDDVPMVLVGNKCDLPTRTVDTKQAHELAKSY |
| | | | GIPFIETSAKTRQGVEDAFYTLVREIRQYRMKKLNSSDDGTQGCMG |
| | | | LPCVVM |
| 3 | TERT | AAD30037.1 | MPRAPRCRAVRSLLRSHYREVLPLATFVRRLGPQGWRLVQRGDPA |
| | | | AFRALVAQCLVCVPWDARPPPAAPSFRQVSCLKELVARVLQRLCER |
| | | | GAKNVLAFGFALLDGARGGPPEAFTTSVRSYLPNTVTDALRGSGA |
| | | | WGLLLRRVGDDVLVHLLARCALFVLVAPSCAYQVCGPPLYQLGAA |
| | | | TQARPPPHASGPRRRLGCERAWNHSVREAGVPLGLPAPGARRRGG |
| | | | SASRSLPLPKRPRRGAAPEPERTPVGQGSWAHPGRTRGPSDRGFCV |
| | | | VSPARPAEEATSLEGALSGTRHSHPSVGRQHHAGPPSTSRPPRPWDT |
| | | | PCPPVYAETKHFLYSSGDKEQLRPSFLLSSLRPSLTGARRLVETIFLG |
| | | | SRPWMPGTPRRLPRLPQRYWQMRPLFLELLGNHAQCPYGVLLKTH |
| | | | CPLRAAVTPAAGVCAREKPQGSVAAPEEEDTDPRRLVQLLRQHSSP |
| | | | WQVYGFVRACLRRLVPPGLWGSRHNERRFLRNTKKFISLGKHAKL |
| | | | SLQELTWKMSVRDCAWLRRSPGVGCVPAAEHRLREEILAKFLHWL |
| | | | MSVYVVELLRSFFYVTETTFQKNRLFFYRKSVWSKLQSIGIRQHLKR |
| | | | VQLRELSEAEVRQHREARPALLTSRLRFIPKPDGLRPIVNMDYVVG |
| | | | ARTFRREKRAERLTSRVKALFSVLNYERARRPGLLGASVLGLDDIH |
| | | | RAWRTFVLRVRAQDPPPELYFVKVDVTGAYDTIPQDRLTEVIASIIK |
| | | | PQNTYCVRRYAVVQKAAHGHVRKAFKSHVSTLTDLQPYMRQFVA |
| | | | HLQETSPLRDAVVIEQSSSLNEASSGLFDVFLRFMCHHAVRIRGKSY |
| | | | VQCQGIPQGSILSTLLCSLCYGDMENKLFAGIRRDGLLLRLVDDFLL |
| | | | VTPHLTHAKTFLRTLVRGVPEYGCVVNLRKTVVNFPVEDEALGGT |
| | | | AFVQMPAHGLFPWCGLLLDTRTLEVQSDYSSYARTSIRASLTFNRG |
| | | | FKAGRNMRRKLFGVLRLKCHSLFLDLQVNSLQTVCTNIYKILLLQA |
| | | | YRFHACVLQLPFHQQVWKNPTFFLRVISDTASLCYSILKAKNAGMS |
| | | | LGAKGAAGPLPSEAVQWLCHQAFLLKLTRHRVTYVPLLGSLRTAQ |
| | | | TQLSRKLPGTTLTALEAAANPALPSDFKTILD |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What we claim is:

1. A method of preparing a microbiome sample for analysis, comprising:
(a) providing one or more adhesive patches, wherein the one or more adhesive patches were applied to a skin surface;
(b) isolating genetic material from the one or more adhesive patches; and
(c) identifying one or more RNA or DNA biomarkers present in the genetic material, wherein the one or more RNA or DNA biomarkers are from a microbe, thereby preparing a microbiome sample for analysis.

2. The method of claim 1, wherein the one or more RNA or DNA biomarkers are from a plurality of microbes.

3. The method of claim 1, wherein the one or more adhesive patches comprises at least 4, 6, 7, 8, or more patches.

4. The method of claim 1, wherein the one or more adhesive patches were applied to more than one skin surface.

5. The method of claim 1, wherein the genetic material is RNA, DNA, or RNA and DNA.

6. The method of claim 1, wherein identifying the one or more DNA or RNA biomarkers comprises using Sanger sequencing, next generation sequencing, single-molecule real-time sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, or +S sequencing.

7. The method of claim 1, wherein the genetic material comprises pathogenic nucleic acids, bacterial nucleic acids, viral nucleic acids, fungal nucleic acids, parasitic nucleic acids, or any combination thereof.

8. The method of claim 1, wherein the microbe comprises a bacterium or a fungus.

9. The method of claim 8, wherein the microbe comprises a bacterium, and the bacterium is of the genus Actinomycetales, Anaerococcus, Bacillales, Bifidobacterium, Enhydrobacter, Finegoldia, Carnobacterium, Coryneobacterium, Lactobacillus, Lactococcus, Leunconostoc, Macrooccus, Micrococcineae, Oenococcus, Pediococcus, Peptoniphilus, Propionibacterium, Salinicoccus, Sphingomonas, Staphylococcus, Strepococcus, Tetragenoccus, or Weissella.

10. The method of claim 8, wherein the microbe comprises a fungus, and the fungus is of the genus Malassezia, Pityrosporum, Aspergillus, Candida, Cryptococcus, Rhodotorula, or Epicoccum.

11. The method of claim 1, wherein identifying comprises determining expression levels of one or more RNA.

12. The method of claim 1, wherein identifying comprises determining a presence or absence of a DNA mutation in at least one gene of interest.

13. A method of preparing a skin sample for analysis, comprising:
providing a non-invasive skin sample, wherein the skin sample was obtained from a subject using one or more adhesive patches;
extracting nucleic acids from the skin sample, wherein the nucleic acids comprise non-microbial nucleic acids and microbial nucleic acids;
detecting an expression level of one or more target genes in the non-microbial nucleic acids from the skin sample; and
detecting a microbiome profile from the microbial nucleic acids.

14. The method of claim 13, wherein the non-microbial nucleic acids comprise genomic DNA, RNA, or both genomic DNA and RNA.

15. The method of claim 13, wherein detecting a microbiome profile comprises identifying a 16S sequence from the microbial nucleic acids or measuring expression levels of one or more microbial genes.

16. The method of claim 13, wherein the microbiome profile is determined by quantification of one or more of: a cell count of one or more microbes, a total cell count of all detected microbes, and a total cell count of all human cells.

17. The method of claim 13, wherein detecting the expression level of one or more target genes or detecting the microbiome profile comprises at least one amplification step.

18. The method of claim 17, wherein the at least one amplification step comprises quantitative PCR (qPCR), self-sustained sequence replication, transcriptional amplification system, Q-Beta Replicase, or rolling circle replication.

19. The method of claim 13, wherein detecting an expression level of one or more target genes or detecting a microbiome profile comprises using qPCR, microarray, or sequencing.

20. The method of claim 13, wherein the method further comprises identifying a skin condition when the expression level of one or more target genes is increased or decreased by at least about 5% compared to a control.

21. The method of claim 13, wherein the method further comprises identifying a skin condition when the expression level of one or more target genes is increased or decreased by at least about 10% compared to a control.

22. The method of claim 13, wherein the skin sample was obtained from the face or forehead.

23. The method of claim 13, wherein the method further comprises identifying, predicting, or treating a disease or condition from the expression level of one or more target genes in the non-microbial sample and the microbiome profile.

24. The method of claim 23, wherein the disease or condition is a skin disease selected from the group consisting of: psoriasis, atopic dermatitis, seborrhoeic dermatitis, and acne.

25. A skin sample collection kit for capturing genetic and/or microbial data from a subject, comprising:
at least one adhesive tape comprising a flexible backing film, an adhesive matrix for non-invasively collecting a skin microbe sample from a subject, and a peelable release sheet for protecting the adhesive matrix prior to skin sample collection;
a storage area for receiving the adhesive tape after skin microbe sample collection; and
a unique barcode associated with the skin sample.
